SEQUENCE LISTING (98 sequences)

SEQ ID NO: 1 - protein, 124 aa, synthetic construct
ALGKKLLQAAARGQDDEVRELLKAGADVNAKDLIGRTPLHLAASIGHLEIVEVLLKAGADVNAKDSRGWTPLHIAAVSGHLEIVEVLLKAGADVNAQDRAGATPADLAAAEGNEDIAEVLQKAA

SEQ ID NO: 2 - protein, 124 aa, synthetic construct
DLGIKLLHAAFQGQDDEVRILLAAGADVNAKDKVGRTPLHRAAQWGHLEIVEVLLKAGADVNAKDHVGWTPLHLAAWRGHLEIVEVLLKAGADVNAQDASGFTPADLAARQGHEDIAEVLQKAA

SEQ ID NO: 3 - protein, 124 aa, synthetic construct
DLGRKLLQAAARGQDDEVRELLKAGADVNAKDLHGRTPLHLAAAIGHLEIVEVLLKAGADVNAKDFLGWTPLHIAAFRGHLEIVEVLLKAGADVNAQDEAGETPADLAAQEGHEDIAEVLQKAA

SEQ ID NO: 4 - protein, 124 aa, synthetic construct
DLGKKLLQAAARGQDDEVRELLKAGADVNAKDLKGRTPLHLAAATGHPEIVEVLLKAGADVNAKDQFGYTPLHIAAAWGHLEIVEVLLKAGADVNAQDQKGETPADLAAKEGHEDIAEVLQKAA

SEQ ID NO: 5 - protein, 124 aa, synthetic construct
DLGRKLLQAAARGQDDEVRELLKAGADVNAKDLLGRTPLHLAASIGHLEIVEVLLKAGADVNAKDERGFTPLHIAALQGHLEIVEVLLKAGADVNAQDLEGRTPADLAAEEGHEDIAEVLQKAA

SEQ ID NO: 6 - protein, 124 aa, synthetic construct
DLGRKLLQAAARGQDDEVRELLKAGADVNAKDLHGRTPLHLAAAIGHLEIVEVLLKAGADVNAKDVFGYTPLHIAAFRGHLEIVEVLLKAGADVNAQDERGETPADLAAEAGHEDIAEVLQKAA

SEQ ID NO: 7 - protein, 124 aa, synthetic construct
DLGEKLLKAAARGQDDEVRELLKAGADVNAKDLLGRTPLHLAASIGHLEIVEVLLKAGADVNAKDERGYTPLHIAAIVGHLEIVEVLLKAGADVNAQDARGATPADLAAREGHEDIAEVLQKAA

SEQ ID NO: 8 - protein, 124 aa, synthetic construct
DLGKKLLQAAARGQDDEVRELLKAGADVNAKDLLGRTPLHLAASIGHLEIVEVLLKAGADVNAKDSRGFTPLHLAAIIGHLEIVEVLLKAGADVNAQDLKGLTPADLAAAEGHEDIAEVLQKAA

SEQ ID NO: 9 - protein, 33 aa, synthetic construct
KDLIGRTPLHLAASIGHLEIVEVLLKAGADVNA

SEQ ID NO: 10 - protein, 33 aa, synthetic construct
KDSRGWTPLHIAAVSGHLEIVEVLLKAGADVNA

SEQ ID NO: 11 - protein, 33 aa, synthetic construct
KDKVGRTPLHRAAQWGHLEIVEVLLKAGADVNA

SEQ ID NO: 12 - protein, 33 aa, synthetic construct
KDHVGWTPLHLAAWRGHLEIVEVLLKAGADVNA

SEQ ID NO: 13 - protein, 33 aa, synthetic construct
KDLHGRTPLHLAAAIGHLEIVEVLLKAGADVNA

SEQ ID NO: 14 - protein, 33 aa, synthetic construct
KDFLGWTPLHIAAFRGHLEIVEVLLKAGADVNA

SEQ ID NO: 15 - protein, 33 aa, synthetic construct
KDLKGRTPLHLAAATGHPEIVEVLLKAGADVNA

SEQ ID NO: 16 - protein, 33 aa, synthetic construct
KDQFGYTPLHIAAAWGHLEIVEVLLKAGADVNA

SEQ ID NO: 17 - protein, 33 aa, synthetic construct
KDLLGRTPLHLAASIGHLEIVEVLLKAGADVNA

SEQ ID NO: 18 - protein, 33 aa, synthetic construct
KDERGFTPLHIAALQGHLEIVEVLLKAGADVNA

SEQ ID NO: 19 - protein, 33 aa, synthetic construct
KDVFGYTPLHIAAFRGHLEIVEVLLKAGADVNA

SEQ ID NO: 20 - protein, 33 aa, synthetic construct
KDERGYTPLHIAAIVGHLEIVEVLLKAGADVNA

SEQ ID NO: 21 - protein, 33 aa, synthetic construct
KDSRGFTPLHLAAIIGHLEIVEVLLKAGADVNA

SEQ ID NO: 22 - protein, 30 aa, synthetic construct
ALGKKLLQAAARGQDDEVRELLKAGADVNA

SEQ ID NO: 23 - protein, 30 aa, synthetic construct
ALGKKLLQAAARGQDDEVRILLKAGADVNA

SEQ ID NO: 24 - protein, 30 aa, synthetic construct
ALGKKLLQAAARGQDDEVRILLAAGADVNA

SEQ ID NO: 25 - protein, 30 aa, synthetic construct
DLGIKLLHAAFQGQDDEVRILLAAGADVNA

SEQ ID NO: 26 - protein, 30 aa, synthetic construct
DLGIKLLHAAFQGQDDEVRELLKAGADVNA

SEQ ID NO: 27 - protein, 30 aa, synthetic construct
DLGIKLLHAAFQGQDDEVRILLKAGADVNA

SEQ ID NO: 28 - protein, 30 aa, synthetic construct
DLGRKLLQAAARGQDDEVRELLKAGADVNA

SEQ ID NO: 29 - protein, 30 aa, synthetic construct
DLGRKLLQAAARGQDDEVRILLAAGADVNA

SEQ ID NO: 30 - protein, 30 aa, synthetic construct
DLGRKLLQAAARGQDDEVRILLKAGADVNA

SEQ ID NO: 31 - protein, 30 aa, synthetic construct
DLGKKLLQAAARGQDDEVRELLKAGADVNA

SEQ ID NO: 32 - protein, 30 aa, synthetic construct
DLGKKLLQAAARGQDDEVRILLAAGADVNA

SEQ ID NO: 33 - protein, 30 aa, synthetic construct
DLGKKLLQAAARGQDDEVRILLKAGADVNA

SEQ ID NO: 34 - protein, 30 aa, synthetic construct
DLGEKLLKAAARGQDDEVRELLKAGADVNA

SEQ ID NO: 35 - protein, 30 aa, synthetic construct
DLGEKLLKAAARGQDDEVRILLAAGADVNA

SEQ ID NO: 36 - protein, 30 aa, synthetic construct
DLGEKLLKAAARGQDDEVRILLKAGADVNA

SEQ ID NO: 37 - protein, 28 aa, synthetic construct
QDRAGATPADLAAAEGNEDIAEVLQKAA

SEQ ID NO: 38 - protein, 28 aa, synthetic construct
QDRAGATPADLAAAEGNEDIAEVLQKLA

SEQ ID NO: 39 - protein, 28 aa, synthetic construct
QDRAGATPADLAAAEGNEDIAEVLQKLN

SEQ ID NO: 40 - protein, 28 aa, synthetic construct
QDASGFTPADLAARQGHEDIAEVLQKAA

SEQ ID NO: 41 - protein, 28 aa, synthetic construct
QDASGFTPADLAARQGHEDIAEVLQKLA

SEQ ID NO: 42 - protein, 28 aa, synthetic construct
QDASGFTPADLAARQGHEDIAEVLQKLN

SEQ ID NO: 43 - protein, 28 aa, synthetic construct
QDEAGETPADLAAQEGHEDIAEVLQKAA

SEQ ID NO: 44 - protein, 28 aa, synthetic construct
QDEAGETPADLAAQEGHEDIAEVLQKLA

SEQ ID NO: 45 - protein, 28 aa, synthetic construct
QDEAGETPADLAAQEGHEDIAEVLQKLN

SEQ ID NO: 46 - protein, 28 aa, synthetic construct
QDQKGETPADLAAKEGHEDIAEVLQKAA

SEQ ID NO: 47 - protein, 28 aa, synthetic construct
QDQKGETPADLAAKEGHEDIAEVLQKLA

SEQ ID NO: 48 - protein, 28 aa, synthetic construct
QDQKGETPADLAAKEGHEDIAEVLQKLN

SEQ ID NO: 49 - protein, 28 aa, synthetic construct
QDLEGRTPADLAAEEGHEDIAEVLQKAA

SEQ ID NO: 50 - protein, 28 aa, synthetic construct
QDLEGRTPADLAAEEGHEDIAEVLQKLA

SEQ ID NO: 51 - protein, 28 aa, synthetic construct
QDLEGRTPADLAAEEGHEDIAEVLQKLN

SEQ ID NO: 52 - protein, 28 aa, synthetic construct
QDERGETPADLAAEAGHEDIAEVLQKAA

SEQ ID NO: 53 - protein, 28 aa, synthetic construct
QDERGETPADLAAEAGHEDIAEVLQKLA

SEQ ID NO: 54 - protein, 28 aa, synthetic construct
QDERGETPADLAAEAGHEDIAEVLQKLN

SEQ ID NO: 55 - protein, 28 aa, synthetic construct
QDARGATPADLAAREGHEDIAEVLQKAA

SEQ ID NO: 56 - protein, 28 aa, synthetic construct
QDARGATPADLAAREGHEDIAEVLQKLA

SEQ ID NO: 57 - protein, 28 aa, synthetic construct
QDARGATPADLAAREGHEDIAEVLQKLN

SEQ ID NO: 58 - protein, 28 aa, synthetic construct
QDLKGLTPADLAAAEGHEDIAEVLQKAA

SEQ ID NO: 59 - protein, 28 aa, synthetic construct
QDLKGLTPADLAAAEGHEDIAEVLQKLA

SEQ ID NO: 60 - protein, 28 aa, synthetic construct
QDLKGLTPADLAAAEGHEDIAEVLQKLN

SEQ ID NO: 61 - protein, 12 aa, synthetic construct
MRGSHHHHHHGS

SEQ ID NO: 62 - protein, 124 aa, synthetic construct
DLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 63 - protein, 124 aa, synthetic construct
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 64 - protein, 124 aa, synthetic construct
DLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 65 - protein, 22 aa, synthetic construct
GSPTPTPTTPTPTPTTPTPTPT

SEQ ID NO: 66 - protein, 5 aa, synthetic construct
  VAR_SEQ 1..5: note = n times, wherein n is 1, 2, 3, 4, 5, or 6
GGGGS

SEQ ID NO: 67 - protein, 358 aa, synthetic construct
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 272 aa, synthetic construct
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGRKLLQAAARGQDDEVRELLKAGADVNAKDLHGRTPLHLAAAIGHLEIVEVLLKAGADVNAKDVFGYTPLHIAAFRGHLEIVEVLLKAGADVNAQDERGETPADLAAEAGHEDIAEVLQKAA

SEQ ID NO: 69 - protein, 272 aa, synthetic construct
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGKKLLQAAARGQDDEVRELLKAGADVNAKDLKGRTPLHLAAATGHPEIVEVLLKAGADVNAKDQFGYTPLHIAAAWGHLEIVEVLLKAGADVNAQDQKGETPADLAAKEGHEDIAEVLQKAA

SEQ ID NO: 70 - protein, 272 aa, synthetic construct
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGEKLLKAAARGQDDEVRELLKAGADVNAKDLLGRTPLHLAASIGHLEIVEVLLKAGADVNAKDERGYTPLHIAAIVGHLEIVEVLLKAGADVNAQDARGATPADLAAREGHEDIAEVLQKAA

SEQ ID NO: 71 - protein, 272 aa, synthetic construct
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGRKLLQAAARGQDDEVRELLKAGADVNAKDLLGRTPLHLAASIGHLEIVEVLLKAGADVNAKDERGFTPLHIAALQGHLEIVEVLLKAGADVNAQDLEGRTPADLAAEEGHEDIAEVLQKAA

SEQ ID NO: 72 - protein, 272 aa, synthetic construct
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGKKLLQAAARGQDDEVRELLKAGADVNAKDLLGRTPLHLAASIGHLEIVEVLLKAGADVNAKDSRGFTPLHLAAIIGHLEIVEVLLKAGADVNAQDLKGLTPADLAAAEGHEDIAEVLQKAA

SEQ ID NO: 73 - protein, 372 aa, synthetic construct
GCCCTGGGTAAGAAGCTGCTGCAAGCTGCTGCTCGTGGTCAGGATGATGAAGTTCGTGAACTGCTGAAAGCAGGCGCCGATGTTAATGCAAAAGATCTGATCGGCAGAACCCCGCTGCATCTGGCTGCTTCTATCGGTCACCTGGAAATTGTTGAAGTTCTGCTGAAAGCCGGTGCAGATGTTAATGCAAAAGATTCTAGAGGCTGGACCCCGCTGCATATCGCTGCTGTGTCTGGTCACCTGGAAATTGTTGAAGTTCTGCTGAAAGCCGGTGCAGATGTTAACGCACAGGATCGTGCTGGCGCTACCCCTGCCGATCTGGCAGCTGCTGAAGGTAATGAAGATATTGCAGAAGTGCTGCAGAAGGCAGCG

SEQ ID NO: 74 - protein, 372 aa, synthetic construct
GACCTGGGTATCAAACTGCTGCATGCTGCTTTCCAGGGTCAGGACGACGAAGTTCGTATCCTGCTGGCTGCTGGCGCCGACGTTAATGCTAAAGACAAAGTTGGTCGTACTCCGCTGCACCGTGCTGCTCAGTGGGGTCACCTGGAAATCGTTGAAGTTCTACTGAAGGCTGGTGCTGACGTTAATGCTAAAGACCATGTTGGTTGGACTCCGCTGCACCTGGCTGCTTGGCGTGGTCACCTGGAAATCGTTGAAGTTCTGCTGAAGGCTGGTGCTGACGTTAACGCTCAGGACGCTTCTGGTTTCACTCCGGCTGATCTGGCTGCTCGTCAGGGTCACGAGGATATCGCTGAAGTTCTGCAGAAGGCAGCG

SEQ ID NO: 75 - protein, 372 aa, synthetic construct
GACCTGGGTCGTAAGCTGCTGCAAGCTGCTGCTCGTGGTCAGGATGATGAAGTTCGTGAACTGCTGAAAGCAGGCGCCGATGTTAATGCAAAAGATCTGCACGGCAGAACCCCGCTGCATCTGGCTGCTGCTATCGGTCACCTGGAAATTGTTGAAGTTCTGCTGAAAGCCGGTGCAGATGTTAATGCAAAAGATTTCCTGGGCTGGACCCCGCTGCATATCGCTGCTTTCAGAGGTCACCTGGAAATTGTTGAAGTTCTGCTGAAAGCCGGTGCAGATGTTAACGCACAGGATGAAGCTGGCGAAACCCCTGCCGATCTGGCAGCTCAGGAAGGTCATGAAGATATTGCAGAAGTGCTGCAGAAGGCAGCG

SEQ ID NO: 76 - protein, 372 aa, synthetic construct
GACCTGGGTAAGAAGCTGCTGCAAGCTGCTGCTCGTGGTCAGGATGATGAAGTTCGTGAACTGCTGAAAGCAGGCGCCGATGTTAATGCAAAAGATCTGAAGGGCAGAACCCCGCTGCATCTGGCTGCTGCTACCGGTCACCCGGAAATTGTTGAAGTTCTGCTGAAAGCCGGTGCAGATGTTAATGCAAAAGATCAATTCGGCTACACCCCGCTGCATATCGCTGCTGCTTGGGGTCACCTGGAAATTGTTGAAGTTCTGCTGAAAGCCGGTGCAGATGTTAACGCACAGGATCAGAAGGGCGAAACCCCTGCCGATCTGGCAGCTAAGGAAGGTCATGAAGATATTGCAGAAGTGCTGCAGAAGGCAGCG

SEQ ID NO: 77 - protein, 372 aa, synthetic construct
GACCTGGGTCGTAAGCTGCTGCAAGCTGCTGCTCGTGGTCAGGATGATGAAGTTCGTGAACTGCTGAAAGCAGGCGCCGATGTTAATGCAAAAGATCTGCTGGGCAGAACCCCGCTGCATCTGGCTGCTTCTATCGGTCACCTGGAAATTGTTGAAGTTCTGCTGAAAGCCGGTGCAGATGTTAATGCAAAAGATGAGAGAGGCTTCACCCCGCTGCATATCGCTGCTTTACAAGGTCACCTGGAAATTGTTGAAGTTCTGCTGAAAGCCGGTGCAGATGTTAACGCACAGGATCTGGAAGGCCGTACCCCTGCCGATCTGGCAGCTGAAGAAGGTCATGAAGATATTGCAGAAGTGCTGCAGAAGGCAGCG

SEQ ID NO: 78 - protein, 372 aa, synthetic construct
GACCTGGGTCGTAAGCTGCTGCAAGCTGCTGCTCGTGGTCAGGATGATGAAGTTCGTGAACTGCTGAAAGCAGGCGCCGATGTTAATGCAAAAGATCTGCACGGCAGAACCCCGCTGCATCTGGCTGCTGCTATCGGTCACCTGGAAATTGTTGAAGTTCTGCTGAAAGCCGGTGCAGATGTTAATGCAAAAGATGTGTTCGGCTACACCCCGCTGCATATCGCTGCCTTCAGAGGTCACCTGGAAATTGTTGAAGTTCTGCTGAAAGCCGGTGCAGATGTTAACGCACAGGATGAACGTGGCGAAACCCCTGCCGATCTGGCAGCTGAAGCTGGTCATGAAGATATTGCAGAAGTGCTGCAGAAGGCAGCG

SEQ ID NO: 79 - protein, 372 aa, synthetic construct
GACCTGGGTGAAAAGCTGCTGAAGGCTGCTGCTCGTGGTCAGGATGATGAAGTTCGTGAACTGCTGAAAGCAGGCGCCGATGTTAATGCAAAAGATCTGCTGGGCAGAACCCCGCTGCATCTGGCTGCTTCTATCGGTCACCTGGAAATTGTTGAAGTTCTGCTGAAAGCCGGTGCAGATGTTAATGCAAAAGATGAGAGAGGCTACACCCCGCTGCATATCGCTGCTATCGTGGGTCACCTGGAAATTGTTGAAGTTCTGCTGAAAGCCGGTGCAGATGTTAACGCACAGGATGCTCGTGGCGCTACCCCTGCCGATCTGGCAGCTCGTGAAGGTCATGAAGATATTGCAGAAGTGCTGCAGAAGGCAGCG

SEQ ID NO: 80 - protein, 372 aa, synthetic construct
GACCTGGGTAAGAAGCTGCTGCAAGCTGCTGCTCGTGGTCAGGATGATGAAGTTCGTGAACTGCTGAAAGCAGGCGCCGATGTTAATGCAAAAGATCTGCTGGGCAGAACCCCGCTGCATCTGGCTGCTTCTATCGGTCACCTGGAAATTGTTGAAGTTCTGCTGAAAGCCGGTGCAGATGTTAATGCAAAAGATTCTAGAGGCTTCACCCCGCTGCATCTGGCTGCTATCATCGGTCACCTGGAAATTGTTGAAGTTCTGCTGAAAGCCGGTGCAGATGTTAACGCACAGGATCTGAAGGGCCTGACCCCTGCCGATCTGGCAGCTGCTGAAGGTCATGAAGATATTGCAGAAGTGCTGCAGAAGGCAGCG

SEQ ID NO: 81 - protein, 124 aa, synthetic construct
DLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 82 - protein, 30 aa, synthetic construct
  VARIANT 1..30: note = wherein X is selected from any amino acid
DLGXKLLQAAXXGQLDXVRXLLXAGADVNA

SEQ ID NO: 83 - protein, 30 aa, synthetic construct
  VARIANT 1..30: note = wherein X is selected from any amino acid
DLGXXLLQAAXXGQLDXVRXLXXXGADVNA

SEQ ID NO: 84 - protein, 30 aa, synthetic construct
  VARIANT 1..30: note = wherein X is selected from anz amino acid
XXXXXLLQAAXXGXXXXXXXXXXXGADVNA

SEQ ID NO: 85 - protein, 30 aa, synthetic construct
  VARIANT 1..30: note = wherein X is selected from any amino acid
DLGXKLLEAAXXGQDDXVRXLLXAGADVNA

SEQ ID NO: 86 - protein, 30 aa, synthetic construct
  VARIANT 1..30: note = wherein X is selected from any amino acid
DLGXXLLEAAXXGQDDXVRXLXXXGADVNA

SEQ ID NO: 87 - protein, 30 aa, synthetic construct
  VARIANT 1..30: note = wherein X is selected from any amino acid
XXXXXLLXAAXXGXDXXXXXXXXXGADVNA

SEQ ID NO: 88 - protein, 28 aa, synthetic construct
  VARIANT 1..28: note = wherein X is selected from any amino acid
XDXXGXTPADXAADXGHEXIAXVLQXAA

SEQ ID NO: 89 - protein, 124 aa, synthetic construct
DLGQKLLKAAQYGQDDEVRILLAAGADVNAKDWAGKTPLHVAAEAGHLEIVEVLLKAGADVNAKDKAGETPLHLAAEQGHLEIVEVLLKAGADVNAQDEWGQTPADLAAYYGLEDIAEVLQKAA

SEQ ID NO: 90 - protein, 124 aa, synthetic construct
DLGAKLLKAAQSGQDDEVRILLAAGADVNAKDKDGYTPLHLAAREGHLEIVEVLLKAGSDVNAKDYYGETPLHQAAEYGHLEIVEVLLKAGADVNAQDEWGATPADLAAQQGHEDIAEVLQKAA

SEQ ID NO: 91 - protein, 124 aa, synthetic construct
DLGKKLLQAARAGQLDEVRELLKAGADVNAKDVYGLTPLHLAAREGHLEIVEVLLKAGADVNAKDAYGETPLHLAAEAGHLEIVEVLLKAGADVNAKDEWGETPLNVAARAGHQDIAEVLQKAA

SEQ ID NO: 92 - protein, 124 aa, synthetic construct
DLGKKLLKAASWGQDDEVRILLAAGADVNAKDNSGWTPLHAAAWYGHLEIVEVLLKAGADVNAKDKTGLTPLHKAAHVGHLEIVEVLLKAGADVNAQDEWGATPADLAASTGHEDIAEVLQKAA

SEQ ID NO: 93 - protein, 124 aa, synthetic construct
DLGHKLLHAAAWGQDDEVRILLAAGADVNAKDQSGWTPLHVAAWYGHLEIVEVLLKAGADVNAKDLKGETPLHLAALVGHLEIVEVLLKAGADVNAQDIAGDTPADLAAIAGHEDIAEVLQKAA

SEQ ID NO: 94 - protein, 434 aa, synthetic construct
DLGKKLLQAAARGQDDEVRELLKAGADVNAKDLLGRTPLHLAASIGHLEIVEVLLKAGADVNAKDSRGFTPLHLAAIIGHLEIVEVLLKAGADVNAQDLKGLTPADLAAAEGHEDIAEVLQKAAGSPTPTPTTPTPTPTTPTPTPTTPTPTPTTPTPTPTGSDLGQKLLKAAQYGQDDEVRILLAAGADVNAKDWAGKTPLHVAAEAGHLEIVEVLLKAGADVNAKDKAGETPLHLAAEQGHLEIVEVLLKAGADVNAQDEWGQTPADLAAYYGLEDIAEVLQKAAGSPTPTPTTPTPTPTTPTPTPTGSDLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 95 - protein, 434 aa, synthetic construct
DLGKKLLQAAARGQDDEVRELLKAGADVNAKDLLGRTPLHLAASIGHLEIVEVLLKAGADVNAKDSRGFTPLHLAAIIGHLEIVEVLLKAGADVNAQDLKGLTPADLAAAEGHEDIAEVLQKAAGSPTPTPTTPTPTPTTPTPTPTTPTPTPTTPTPTPTGSDLGAKLLKAAQSGQDDEVRILLAAGADVNAKDKDGYTPLHLAAREGHLEIVEVLLKAGSDVNAKDYYGETPLHQAAEYGHLEIVEVLLKAGADVNAQDEWGATPADLAAQQGHEDIAEVLQKAAGSPTPTPTTPTPTPTTPTPTPTGSDLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 96 - protein, 434 aa, synthetic construct
DLGKKLLQAAARGQDDEVRELLKAGADVNAKDLLGRTPLHLAASIGHLEIVEVLLKAGADVNAKDSRGFTPLHLAAIIGHLEIVEVLLKAGADVNAQDLKGLTPADLAAAEGHEDIAEVLQKAAGSPTPTPTTPTPTPTTPTPTPTTPTPTPTTPTPTPTGSDLGKKLLQAARAGQLDEVRELLKAGADVNAKDVYGLTPLHLAAREGHLEIVEVLLKAGADVNAKDAYGETPLHLAAEAGHLEIVEVLLKAGADVNAKDEWGETPLNVAARAGHQDIAEVLQKAAGSPTPTPTTPTPTPTTPTPTPTGSDLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 97 - protein, 434 aa, synthetic construct
DLGKKLLQAAARGQDDEVRELLKAGADVNAKDLLGRTPLHLAASIGHLEIVEVLLKAGADVNAKDSRGFTPLHLAAIIGHLEIVEVLLKAGADVNAQDLKGLTPADLAAAEGHEDIAEVLQKAAGSPTPTPTTPTPTPTTPTPTPTTPTPTPTTPTPTPTGSDLGKKLLKAASWGQDDEVRILLAAGADVNAKDNSGWTPLHAAAWYGHLEIVEVLLKAGADVNAKDKTGLTPLHKAAHVGHLEIVEVLLKAGADVNAQDEWGATPADLAASTGHEDIAEVLQKAAGSPTPTPTTPTPTPTTPTPTPTGSDLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

SEQ ID NO: 98 - protein, 434 aa, synthetic construct
DLGKKLLQAAARGQDDEVRELLKAGADVNAKDLLGRTPLHLAASIGHLEIVEVLLKAGADVNAKDSRGFTPLHLAAIIGHLEIVEVLLKAGADVNAQDLKGLTPADLAAAEGHEDIAEVLQKAAGSPTPTPTTPTPTPTTPTPTPTTPTPTPTTPTPTPTGSDLGHKLLHAAAWGQDDEVRILLAAGADVNAKDQSGWTPLHVAAWYGHLEIVEVLLKAGADVNAKDLKGETPLHLAALVGHLEIVEVLLKAGADVNAQDIAGDTPADLAAIAGHEDIAEVLQKAAGSPTPTPTTPTPTPTTPTPTPTGSDLGQKLLRAAKKGQDDEVRELLKAGADVNAKDEQGWTPLHIAAWKGHLEIVEVLLKAGADVNAKDVFDWTPLHVAAHFGHLEIVEVLLKAGADVNAQDRAGETPADLAAQAGHEDIAEVLQKAA

(19) United States
(12) Patent Application Publication
(10) Pub. No.: US 2026/0297190 A1
(43) Pub. Date: Oct. 1, 2026

(54) RECOMBINANT CD47 BINDING PROTEINS AND THEIR USE

(71) Applicant: MOLECULAR PARTNERS AG, Zürich-Schlieren (CH)

(72) Inventors: Alexander LINK, Zürich-Schlieren (CH); Valerie Perrine CALABRO, Zürich-Schlieren (CH); Anja Christina SCHLEGEL, Zürich-Schlieren (CH); Natalia VENETZ ARENAS, Zürich-Schlieren (CH)

(21) Appl. No.: 19/489,795

(22) PCT Filed: Jun. 4, 2024
(86) PCT No.: PCT/EP2024/065257

§ 371 Date: Dec. 3, 2025

(30) Foreign Application Priority Data
Jun. 6, 2023 (EP) 23177836.6

(51) Int. Cl. C07K 16/28; A61K 39/00; A61P 35/00; C07K 16/30

(52) U.S. Cl. CPC C07K 16/2803; A61P 35/00; C07K 16/283; C07K 16/30; A61K 2039/505; C07K 2317/31; C07K 2317/52; C07K 2317/90; C07K 2319/21; C07K 2319/30; C07K 2319/43; C07K 2319/70

(57) ABSTRACT

The present invention relates to recombinant binding proteins comprising a designed ankyrin repeat domain with binding specificity for CD47. In addition, the invention relates to nucleic acids encoding such recombinant binding proteins, pharmaceutical compositions comprising such recombinant binding proteins or nucleic acids, and the use of such recombinant binding proteins, nucleic acids or pharmaceutical compositions in methods of treating diseases, such as cancer, in a mammal, including a human.

### FIELD OF THE DISCLOSURE

The present invention relates to recombinant binding proteins comprising a designed ankyrin repeat domain with binding specificity for CD47. In addition, the invention relates to nucleic acids encoding such binding proteins, pharmaceutical compositions comprising such binding proteins or nucleic acids, and the use of such binding proteins, nucleic acids or pharmaceutical compositions in methods of treating diseases, such as cancer, in a mammal, including a human.

### BACKGROUND

The majority of currently developed immunotherapies works primarily by modulating adaptive immunity, especially by revitalizing and boosting T cell responses. However, emerging studies have shown that innate immune checkpoint molecules expressed on certain antigen-presenting cells (APCs) play a crucial role in immune evasion. Such cells can detect and eliminate cancer cells by phagocytosis and the expressed checkpoint molecules can inhibit this innate immune response. Innate immune cells, including macrophages, monocytes, dendritic cells (DCs), and natural killer (NK) cells, are the first line of the immune defense system. They establish proinflammatory responses to foreign invaders and repair damaged tissues (Liu et al; Signal Transduct Target Ther; 2023 Mar. 7; 8 (1): 104). Cancer cells can evade clearance by macrophages via overexpressing anti-phagocytic membrane proteins causing so-called “don't eat me” signals, including cluster of differentiation 47 (CD47) (Zhang et al: Front Immunol; 2020 Jan. 28; 11:18).

CD47 is a cell surface glycoprotein, which includes an amino terminal extracellular variable region, a transmembrane region formed of a highly hydrophobic transmembrane segment, and a hydrophilic carboxy-terminal cytoplasmic tail. It functions as a regulator of phagocytosis mediated by cells of the innate immune system by interacting with multiple ligands, such as integrins, signal regulatory protein alpha (SIRPa), signal regulatory protein gamma (SIRPy) and thrombospondins. In particular, CD47 inhibits phagocytosis by interacting with the transmembrane protein SIRPa on the surface of phagocytes, such as macrophages, neutrophils and dendritic cells, forming the CD47-SIRPa signaling complex and triggering a “don't eat me” signal.

Numerous studies have shown that CD47 is overexpressed in different types of tumors, including in myeloma, leiomyosarcoma, acute lymphocytic leukaemia, non-Hodgkin's lymphoma, breast cancer, osteosarcoma, and head and neck squamous cell carcinoma (Zhang et al: Front Immunol; 2020 Jan. 28; 11:18).

Through upregulated expression of CD47, tumor cells manage to evade phagocytosis and escape from innate immune surveillance and therefore CD47 shows a remarkable potential as a therapeutic target. However, CD47 is also broadly expressed on normal cells, such as hematopoietic cells, red blood cells (RBCs) and platelets, thus posing safety and efficacy challenges, as targeting CD47, for example with a neutralizing antibody, could affect healthy cells, potentially leading to toxic effects (Ye at al; Acta Pharm Sin B. 2023 April; 13 (4): 1467-1487). Additionally, broad expression of CD47 could also lead to rapid elimination of CD47 binding agents, leading to poor pharmacokinetics and decreased efficacy (Dizman et al; Cancers (Basel). 2021 December; 13 (24): 6229).

Many of the anti-CD47 antibodies that have been reported are known to cause agglutination of RBCs upon binding to CD47 on the cell surface, which significantly hampers the therapeutic utility of such antibodies (Velliquette et al; Transfusion; 2019 February; 59 (2): 730-737; Sun et al; Leuk Res Rep; 2021 Sep. 14; 16).

In addition to playing a critical role in cancer and the tumor microenvironment, CD47 is implicated in many other diseases. For example, in pulmonary arterial hypertension (PAH) the levels of CD47 are increased and promote hypoxia and ROS production in the environment (Novelli et al; Am. J. Physiol. Lung Cell Mol. Physiol. 316, L1150-L1164 (2019)). Activated CD47 was also found to promote acute kidney injury (AKI) by limiting autophagy, and has been demonstrated to be a target for preserving renal function following injury (E1-Rashid et al; FASEB J. 33, 12735-12749 (2019)). Furthermore, CD47 mediates immune escape in infectious diseases caused by parasites, bacteria, and viruses, including SARS-COV-2 in COVID pathogenesis (Cham et al; Antibodies; 9, 44 (2020)).

Taken together, there remains a need for new diagnostic and/or therapeutic approaches and agents benefitting from CD47-specific binding and, optionally, inhibition of interaction between SIRPα and CD47.

### SUMMARY

The present invention provides recombinant binding proteins comprising a designed ankyrin repeat domain with binding specificity for CD47. Further provided are such recombinant binding proteins linked to one or more binding agents, preferably ankyrin repeat domains, with binding specificity for a protein expressed on the surface of an immune cell, for human serum albumin and/or for a further Tumor Associated Antigen (TAA). In addition, the invention provides nucleic acids encoding such recombinant binding proteins and pharmaceutical compositions comprising such recombinant binding proteins or nucleic acids. The invention also provides the use of such recombinant binding proteins, nucleic acids or pharmaceutical compositions in methods of treating diseases, such as cancer, in a mammal, including a human.

Applicant identified, designed, and generated CD47-specific binding domains, which are suitable to be used as building blocks in diagnostic or therapeutic agents, such as, e.g., multi-specific binding proteins, and have beneficial properties. As the result of a complex screening process and rational design, multiple recombinant CD47-specific ankyrin repeat proteins were generated and produced, namely DARPin proteins comprising SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8. These recombinant binding proteins all comprise a designed ankyrin repeat domain with binding specificity for CD47. These recombinant binding proteins are suitable to be linked to one or more Tumor Associated Antigen (TAA)-specific binding agent(s), which may result in avidity driven multi-specific molecules. Moreover, binding proteins of the invention may further be linked to other binding agents, such as binding agents with binding specificity for surface molecules expressed by immune cells.

Based on the disclosure provided herein, those skilled in the art will recognize, or be able to ascertain using no more than routine experimentation, many equivalents to the specific embodiments of the invention described herein. Such equivalents are intended to be encompassed by the following numbered embodiments (E).
E1. A recombinant binding protein comprising an ankyrin repeat domain with binding specificity for CD47, wherein said ankyrin repeat domain comprises an ankyrin repeat module having an amino acid sequence selected from the group consisting of (1) SEQ ID NOs: 9 to 21 and (2) sequences in which up to 9 amino acids in any of SEQ ID NOs: 9 to 21 are substituted by other amino acids.
E2. The recombinant binding protein of E1, wherein said ankyrin repeat module is a first ankyrin repeat module and wherein said ankyrin repeat domain further comprises a second ankyrin repeat module having an amino acid sequence selected from the group consisting of (1) SEQ ID NOs: 9 to 21 and (2) sequences in which up to 9 amino acids in any of SEQ ID NOs: 9 to 21 are substituted by other amino acids.
E3. The recombinant binding protein of E2, wherein said first ankyrin repeat module comprises an amino acid sequence selected from the group consisting of (1) SEQ ID NO: 9 and (2) sequences in which up to 9 amino acids in SEQ ID NO: 9 are substituted by other amino acids, and wherein said second ankyrin repeat module comprises an amino acid sequence selected from the group consisting of (1) SEQ ID NO: 10 and (2) sequences in which up to 9 amino acids of SEQ ID NO: 10 are substituted by other amino acids.
E4. The recombinant binding protein of E2, wherein said first ankyrin repeat module comprises an amino acid sequence selected from the group consisting of (1) SEQ ID NO: 11 and (2) sequences in which up to 9 amino acids in SEQ ID NO: 11 are substituted by other amino acids, and wherein said second ankyrin repeat module comprises an amino acid sequence selected from the group consisting of (1) SEQ ID NO: 12 and (2) sequences in which up to 9 amino acids of SEQ ID NO: 12 are substituted by other amino acids.
E5. The recombinant binding protein of E2, wherein said first ankyrin repeat module comprises an amino acid sequence selected from the group consisting of (1) SEQ ID NO: 13 and (2) sequences in which up to 9 amino acids in SEQ ID NO: 13 are substituted by other amino acids, and wherein said second ankyrin repeat module comprises an amino acid sequence selected from the group consisting of (1) SEQ ID NO: 14 and (2) sequences in which up to 9 amino acids of SEQ ID NO: 14 are substituted by other amino acids.
E6. The recombinant binding protein of E2, wherein said first ankyrin repeat module comprises an amino acid sequence selected from the group consisting of (1) SEQ ID NO: 15 and (2) sequences in which up to 9 amino acids in SEQ ID NO: 15 are substituted by other amino acids, and wherein said second ankyrin repeat module comprises an amino acid sequence selected from the group consisting of (1) SEQ ID NO: 16 and (2) sequences in which up to 9 amino acids of SEQ ID NO: 16 are substituted by other amino acids.
E7. The recombinant binding protein of E2, wherein said first ankyrin repeat module comprises an amino acid sequence selected from the group consisting of (1) SEQ ID NO: 17 and (2) sequences in which up to 9 amino acids in SEQ ID NO: 17 are substituted by other amino acids, and wherein said second ankyrin repeat module comprises an amino acid sequence selected from the group consisting of (1) SEQ ID NO: 18 and (2) sequences in which up to 9 amino acids of SEQ ID NO: 18 are substituted by other amino acids.
E8. The recombinant binding protein of E2, wherein said first ankyrin repeat module comprises an amino acid sequence selected from the group consisting of (1) SEQ ID NO: 13 and (2) sequences in which up to 9 amino acids in SEQ ID NO: 13 are substituted by other amino acids, and wherein said second ankyrin repeat module comprises an amino acid sequence selected from the group consisting of (1) SEQ ID NO: 19 and (2) sequences in which up to 9 amino acids of SEQ ID NO: 19 are substituted by other amino acids.
E9. The recombinant binding protein of E2, wherein said first ankyrin repeat module comprises an amino acid sequence selected from the group consisting of (1) SEQ ID NO: 17 and (2) sequences in which up to 9 amino acids in SEQ ID NO: 17 are substituted by other amino acids, and wherein said second ankyrin repeat module comprises an amino acid sequence selected from the group consisting of (1) SEQ ID NO: 20 and (2) sequences in which up to 9 amino acids of SEQ ID NO: 20 are substituted by other amino acids.
E10. The recombinant binding protein of E2, wherein said first ankyrin repeat module comprises an amino acid sequence selected from the group consisting of (1) SEQ ID NO: 17 and (2) sequences in which up to 9 amino acids in SEQ ID NO: 17 are substituted by other amino acids, and wherein said second ankyrin repeat module comprises an amino acid sequence selected from the group consisting of (1) SEQ ID NO: 21 and (2) sequences in which up to 9 amino acids of SEQ ID NO: 21 are substituted by other amino acids.
E11. The recombinant binding protein of any of E2 to E10, wherein said first ankyrin repeat module is located N-terminally of said second ankyrin repeat module within said ankyrin repeat domain.
E12. A recombinant binding protein comprising an ankyrin repeat domain with binding specificity for CD47, wherein said ankyrin repeat domain comprises an amino acid sequence with at least 80% amino acid sequence identity with any one of SEQ ID NOS: 1 to 8, and wherein A at the second last position of SEQ ID NOS: 1 to 8 is optionally substituted by L, and/or A at the last position of SEQ ID NOS: 1 to 8 is optionally substituted by N.
E13. The recombinant binding protein of any of E1 to E12, wherein said recombinant binding protein binds human CD47 in PBS with a dissociation constant (K_{D}) of or below 10⁻⁷M, of or below 10⁻⁸M, of or below 10⁻⁹M.
E14. The recombinant binding protein of any of E1 to E13, wherein said ankyrin repeat domain with binding specificity for CD47 comprises an amino acid sequence with at least 80% amino acid sequence identity with SEQ ID NO: 1.
E15. The recombinant binding protein of E14, wherein said recombinant binding protein binds human CD47 in PBS with a dissociation constant (K_{D}) of or below 10⁻⁸M or of or below 10⁻⁹M.
E16. The recombinant binding protein of any of E1 to E13, wherein said ankyrin repeat domain with binding specificity for CD47 comprises an amino acid sequence with at least 80% amino acid sequence identity with SEQ ID NO: 2.
E17. The recombinant binding protein of E16, wherein said recombinant binding protein binds human CD47 in PBS with a dissociation constant (K_{D}) of or below about 10⁻⁸M or of or below 10⁻⁹M.
E18. The recombinant binding protein of any of E1 to E13, wherein said ankyrin repeat domain with binding specificity for CD47 comprises an amino acid sequence with at least 80% amino acid sequence identity with SEQ ID NO: 3.
E19. The recombinant binding protein of E18, wherein said recombinant binding protein binds human CD47 in PBS with a dissociation constant (K_{D}) of or below about 10⁻⁸M or of or below 10⁻⁹M.
E20. The recombinant binding protein of any of E1 to E13, wherein said ankyrin repeat domain with binding specificity for CD47 comprises an amino acid sequence with at least 80% amino acid sequence identity with SEQ ID NO: 4.
E21. The recombinant binding protein of E20, wherein said recombinant binding protein binds human CD47 in PBS with a dissociation constant (K_{D}) of or below 10⁻⁸M or of or below 10⁻⁹M.
E22. The recombinant binding protein of any of E1 to E13, wherein said ankyrin repeat domain with binding specificity for CD47 comprises an amino acid sequence with at least 80% amino acid sequence identity with SEQ ID NO: 5.
E23. The recombinant binding protein of E22, wherein said recombinant binding protein binds human CD47 in PBS with a dissociation constant (K_{D}) of or below 10⁻⁸M or of or below 10⁻⁹M.
E24. The recombinant binding protein of any of E1 to E13, wherein said ankyrin repeat domain with binding specificity for CD47 comprises an amino acid sequence with at least 80% amino acid sequence identity with SEQ ID NO: 6.
E25. The recombinant binding protein of E24, wherein said recombinant binding protein binds human CD47 in PBS with a dissociation constant (K_{D}) of or below 10⁻⁸M or of or below 10⁻⁹M.
E26. The recombinant binding protein of any of E1 to E13, wherein said ankyrin repeat domain with binding specificity for CD47 comprises an amino acid sequence with at least 80% amino acid sequence identity with SEQ ID NO: 7.
E27. The recombinant binding protein of E26, wherein said recombinant binding protein binds human CD47 in PBS with a dissociation constant (K_{D}) of or below 10⁻⁸M or of or below 10⁻⁹M.
E28. The recombinant binding protein of any of E1 to E13, wherein said ankyrin repeat domain with binding specificity for CD47 comprises an amino acid sequence with at least 80% amino acid sequence identity with SEQ ID NO: 8.
E29. The recombinant binding protein of E28, wherein said recombinant binding protein binds human CD47 in PBS with a dissociation constant (K_{D}) of or below 10⁻⁸M or of or below 10⁻⁹M.
E30. The recombinant binding protein of any of E1 to E29, wherein said recombinant binding protein further comprises a binding agent with binding specificity for a tumor-associated antigen.
E31. The recombinant binding protein of E30, wherein said binding agent with binding specificity for a tumor-associated antigen is an ankyrin repeat domain.
E32. The recombinant binding protein of any one of E1 to E31, wherein said recombinant binding protein further comprises a binding agent with binding specificity for a protein expressed on the surface of an immune cell, preferably an NK cell or a macrophage.
E33. The recombinant binding protein of E32, wherein said binding agent with binding specificity for a protein expressed on the surface of an immune cell, preferably an NK cell or a macrophage, has binding specificity for CD16a.
E34. The recombinant binding protein of E32 or E33, wherein said binding agent with binding specificity for a protein expressed on the surface of an immune cell is a designed ankyrin repeat protein.
E35. The recombinant binding protein of E34, wherein said binding agent with binding specificity said binding agent with binding specificity for a protein expressed on the surface of an immune cell comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 81.
E36. The recombinant binding protein of any one of E18 to E35, wherein said binding agent with binding specificity for a tumor-associated antigen and/or said binding agent with binding specificity for a protein expressed on the surface of an immune cell, preferably an NK cell or a macrophage, is covalently linked to said ankyrin repeat domain with binding specificity for CD47 with a peptide linker.
E37. The recombinant binding protein of E36, wherein said peptide linker is a proline-threonine rich peptide linker or glycine-serine rich linker.
E38. The recombinant binding protein of E34 to E37, wherein the amino acid sequence of said peptide linker has a length from 1 to 50 amino acids, preferably from 6 to 38 amino acids.
E39. The recombinant binding protein of any of E1 to E38, wherein said recombinant binding protein further comprises a half-life extending moiety.
E40. The recombinant binding protein of E39, wherein said half-life extending moiety comprises a binding agent with binding specificity for human serum albumin.
E41. The recombinant binding protein of E40, wherein said binding agent with binding specificity for human serum albumin is an ankyrin repeat domain comprising an amino acid sequence of any one of SEQ ID NOs: 62 to 64, preferably SEQ ID NO: 63.
E42. The recombinant binding protein of any one of E39 to E41, wherein said half-life extending moiety is located N-terminally of said ankyrin repeat domain with binding specificity for CD47 within said binding protein.
E43. A nucleic acid encoding the recombinant binding protein of any one of E1 to E42.
E44. A cloning or expression vector comprising the nucleic acid of E43 wherein said vector is suitable for the recombinant production of a binding protein as defined in any one of E1 to E42.
E45. The vector of E44, wherein said vector is a DNA vector, an RNA vector, a plasmid, a cosmid, or a viral vector.
E46. A host cell comprising the nucleic acid of E43 or the vector of any one of E44 to E45.
E47. A pharmaceutical composition comprising the recombinant binding protein of any one of E1 to E42 or the nucleic acid of E43, the vector of any one of E44 to E45 or the host cell of E46 and optionally a pharmaceutically acceptable carrier and/or diluent.
E48. The recombinant binding protein of any one of E1 to E42, the nucleic acid of E43 or the pharmaceutical composition of E47 for use in a method of treating a medical condition.
E49. The recombinant binding protein of E48, wherein the medical condition is cancer.
E50. A method of treating a medical condition, the method comprising the step of administering to a subject in need thereof a therapeutically effective amount of the recombinant binding protein of any one of E1 to E42, the nucleic acid of E43 or the pharmaceutical composition of E47.
E51. The method of E50, wherein said medical condition is a cancer.
E52. A method of treating, preventing, and/or alleviating a phagocytic cell dysfunctional disease, disorder, or condition, including one or more symptoms of the phagocytic cell dysfunctional disease, disorder, or condition in a mammal, preferably a human, the method comprising the step of administering to said mammal the recombinant binding protein of any one of E1 to E42 or nucleic acid of E43 or the pharmaceutical composition of E47.
E53. Use of the recombinant binding protein of any one of E1 to E42 or the nucleic acid of E43 or the pharmaceutical composition of E47 for the manufacture of a medicament for the treatment of a medical condition.
E54. The use of E53, wherein said medical condition is a cancer.
E55. A method of diagnosing a medical condition in a mammal, preferably a human, the method comprising the steps of:
   (i) contacting a cell or tissue sample obtained from said mammal with the CD47-specific recombinant binding protein of any one of E1 to E42; and
   (ii) detecting specific binding of said recombinant binding protein to said cell or tissue sample.
E56. A method for the production of protein according to any one of E1 to E42, comprising culturing a host cell according to E46, purifying and recovering said protein.

### BRIEF DESCRIPTION OF THE FIGURES

FIG. 1. Binding of 40 nM Flag-tagged 96-well IMAC purified proteins to 10 nM biotinylated human CD47-Fc in Homogeneous Time Resolved Fluorescence (HTRF). The HTRF signal over PBS is shown for the eight selected CD47-specific designed ankyrin repeat proteins. Columns 1 to 8 show the HTRF signal of DARPin protein #1, DARPin protein #2, DARPin protein #3, DARPin protein #4, DARPin protein #5, DARPin protein #6, DARPin protein #7 and DARPin protein #8, respectively.

FIG. 2 (A-D). SIRP1a competition on cells. Titrated amounts of selected designed ankyrin repeat proteins (96 well purified) or anti-CD47 benchmark antibody control was added to the cells (CD47 presenting ligand cells and SIRP1a signaling cells). Reduction of signal indicates ligand competition. FIG. 2A. DARPin protein #3; FIG. 2B. DARPin protein #4 and DARPin protein #7; FIG. 2C. DARPin protein #1, DARPin protein #2, DARPin protein #5 and DARPin protein #8; FIG. 2D. DARPin protein #6.

FIG. 3 (A-C). Cell binding of 96-well purified designed ankyrin repeat proteins. Titrated amount of selected designed ankyrin repeat proteins was added to CD47-expressing cells for 30 minutes at 4° C. Tested proteins bound to cells were detected with anti-DARPin 1.1.1 antibodies. Median fluorescence intensities obtained are shown. FIG. 3A. Cell binding of DARPin protein #1 and #2; FIG. 3B. Cell binding of DARPin protein #3, #4 and #5; FIG. 3C. Cell binding of DARPin protein #6, #7 and #8.

FIG. 4 (A-C) shows SPR traces obtained for DARPin molecule #1 (FIG. 4A), DARPin molecule #2 (FIG. 4B) and DARPin molecule #3 (FIG. 4C). Dissociation constants (K_{D}) were calculated from the estimated on- and off-rates using standard procedures. K_{D} values of the binding interactions of selected ankyrin repeat proteins with human CD47 were determined to be in the range of 0.1 to 0.8 nM (RU, Resonance Units; s, time in seconds).

FIG. 5. Pharmacokinetic analysis of exemplary CD47-specific binding proteins in female BALB/c mice. The figure shows the group mean serum concentration-time profiles of DARPin protein #9, DARPin protein #10, DARPin protein #11, DARPin protein #12 and DARPin protein #13 in female BALB/c mice (mean±/−max/min, N=3 per group), following single intravenous bolus administration of 1 mg/kg.

FIG. 6. SIRP1a competition on cells. Titrated amounts of CD47-specific binding proteins (SEQ ID NOS: 1 to 7) in a trispecific format, or a benchmark anti-CD47 antibody as a control, were added to the cells (CD47 positive/TAA positive ligand cells and SIRP1a signaling cells). Reduction of signal indicates ligand competition.

FIG. 7 (A-B). SIRP1a competition on cells. Titrated amounts of the CD47-specific binding protein (SEQ ID NO: 8), in monospecific and trispecific format or the benchmark anti-CD47 antibody were added to the SIRP1a signaling cells in the presence of either CD47 positive/TAA negative ligand cells (FIG. 7A) or CD47 positive/TAA positive ligand cells (FIG. 7B). Reduction of signal indicates ligand competition.

FIG. 8. SIRP1a competition on cells. Titrated amounts of selected DARPin proteins with binding specificity for CD47, CD117 and CD16a or the benchmark anti-CD47 antibody were added to the SIRP1a signaling cells in the presence of Kasumi-1 cells. Reduction of signal indicates ligand competition.

### DETAILED DESCRIPTION OF THE INVENTION

As disclosed and exemplified herein, the disclosure provides ankyrin repeat proteins that specifically bind target CD47. Designed ankyrin repeat protein libraries (WO2002/020565; Binz et al., Nat. Biotechnol. 22, 575-582, 2004; Stumpp et al., Drug Discov. Today 13, 695-701, 2008) can be used for the selection of target-specific designed ankyrin repeat domains that bind to their target with high affinity. Such target-specific designed ankyrin repeat domains in turn can be used as valuable components of recombinant binding proteins for the treatment of diseases. Designed ankyrin repeat proteins are a class of binding molecules which have the potential to overcome limitations of monoclonal antibodies, hence allowing novel therapeutic approaches. Such ankyrin repeat proteins may comprise a single designed ankyrin repeat domain, or may comprise a combination of two or more designed ankyrin repeat domains with the same or different target specificities (Stumpp et al., Drug Discov. Today 13, 695-701, 2008; U.S. Pat. No. 9,458,211). Ankyrin repeat proteins comprising only a single designed ankyrin repeat domain are small proteins (14 kDa) which can be selected to bind a given target protein with high affinity and specificity. These characteristics, and the possibility of combining two, three, four or more designed ankyrin repeat domains in one protein, resulting in binding proteins with two, three, four or more different specificities, make designed ankyrin repeat proteins ideal agonistic, antagonistic and/or inhibitory drug candidates and allow for novel drug designs with increased safety profile, including, e.g., multispecific drugs comprising a CD47-specific binding agent and at least one further tumor localizer such as a tumor associated antigen binding agent, and wherein said CD47-specific agent is blocked or masked with a binding agent specific for the CD47-specific agent and wherein said CD47-specific agent is released only when said tumor associated antigen binding agent binds to the tumor associated agent, thus preventing binding of said CD47-specific binding agent to normal CD47-expressing cells. In one embodiment, said recombinant binding protein comprises an ankyrin repeat domain with binding specificity for CD47 as described herein and further comprises an ankyrin repeat domain with dual binding specificity, wherein binding of said ankyrin repeat domain with dual binding specificity to its first and second binding targets is mutually exclusive. Such ankyrin repeat domains with dual binding specificity (also referred to as “2-in-1 repeat domains”) and corresponding production methods are described in PCT/EP2022/085794. In a more particular embodiment, said recombinant binding protein comprises an ankyrin repeat domain with binding specificity for CD47 as described herein and further comprises a second ankyrin repeat domain, wherein the second ankyrin repeat domain is a 2-in-1 repeat domain having a first binding specificity for a TAA and a second binding specificity for said first ankyrin repeat domain having binding specificity for CD47. Such a design enables a conditional TAA-dependent activation of the CD47 binding domain, wherein upon presence of the TAA, the 2-in-1 repeat domain binds the TAA and releases the CD47-specific binding domain from binding to the 2-in-1 repeat domain, thereby releasing the inhibition of the CD47-specific binding domain. Furthermore, such ankyrin repeat proteins can be engineered to carry various effector functions, e.g. cytotoxic agents or half-life extending agents, enabling completely new drug formats. Taken together, designed ankyrin repeat proteins are an example of the next generation of protein therapeutics with the potential to surpass existing antibody drugs.

In one aspect, the invention relates to a recombinant binding protein comprising an ankyrin repeat domain, wherein said ankyrin repeat domain has binding specificity for CD47, and wherein said ankyrin repeat domain comprises an ankyrin repeat module having an amino acid sequence selected from the group consisting of (1) SEQ ID NOs: 9 to 21 and (2) sequences in which up to 9, or up to 8, or up to 7, or up to 6, or up to 5, or up to 4, or up to 3, or up to 2, or up to 1 amino acids in any of SEQ ID NOs: 9 to 21 are substituted by other amino acids. Thus, in one embodiment, said ankyrin repeat module comprises an amino acid sequence selected from the group consisting of (1) SEQ ID NOs: 9 to 21 and (2) sequences in which up to 9 amino acids in any of SEQ ID NOs: 9 to 21 are substituted by other amino acids. In one embodiment, said ankyrin repeat module comprises an amino acid sequence selected from the group consisting of (1) SEQ ID NOs: 9 to 21 and (2) sequences in which up to 8 amino acids in any of SEQ ID NOs: 9 to 21 are substituted by other amino acids. In one embodiment, said ankyrin repeat module comprises an amino acid sequence selected from the group consisting of (1) SEQ ID NOs: 9 to 21 and (2) sequences in which up to 7 amino acids in any of SEQ ID NOS: 9 to 21 are substituted by other amino acids. In one embodiment, said ankyrin repeat module comprises an amino acid sequence selected from the group consisting of (1) SEQ ID NOS: 9 to 21 and (2) sequences in which up to 6 amino acids in any of SEQ ID NOS: 9 to 21 are substituted by other amino acids. In one embodiment, said ankyrin repeat module comprises an amino acid sequence selected from the group consisting of (1) SEQ ID NOS: 9 to 21 and (2) sequences in which up to 5 amino acids in any of SEQ ID NOS: 9 to 21 are substituted by other amino acids. In one embodiment, said ankyrin repeat module comprises an amino acid sequence selected from the group consisting of (1) SEQ ID NOs: 9 to 21 and (2) sequences in which up to 4 amino acids in any of SEQ ID NOs: 9 to 21 are substituted by other amino acids. In one embodiment, said ankyrin repeat module comprises an amino acid sequence selected from the group consisting of (1) SEQ ID NOs: 9 to 21 and (2) sequences in which up to 3 amino acids in any of SEQ ID NOs: 9 to 21 are substituted by other amino acids. In one embodiment, said ankyrin repeat module comprises an amino acid sequence selected from the group consisting of (1) SEQ ID NOS: 9 to 21 and (2) sequences in which up to 2 amino acids in any of SEQ ID NOs: 9 to 21 are substituted by other amino acids. In one embodiment, said ankyrin repeat module comprises an amino acid sequence selected from the group consisting of (1) SEQ ID NOs: 9 to 21 and (2) sequences in which up to 1 amino acid in any of SEQ ID NOs: 9 to 21 is substituted by another amino acid. In one embodiment, all of said 9, 8, 7, 6, 5, 4, 3, 2, or 1 amino acid substitutions occur in framework positions of said ankyrin repeat module(s). In one embodiment, said ankyrin repeat module comprises an amino acid sequence selected from the group consisting of SEQ ID NOs: 9 to 21.

In one embodiment, said ankyrin repeat module comprises the amino acid sequence of SEQ ID NO: 9 or a sequence in which one or two amino acids in SEQ ID NO: 9 are substituted by other amino acids. In one embodiment, said ankyrin repeat module comprises the amino acid sequence of SEQ ID NO: 10 or a sequence in which one or two amino acids in SEQ ID NO: 10 are substituted by other amino acids. In one embodiment, said ankyrin repeat module comprises the amino acid sequence of SEQ ID NO: 11 or a sequence in which one or two amino acids in SEQ ID NO: 11 are substituted by other amino acids. In one embodiment, said ankyrin repeat module comprises the amino acid sequence of SEQ ID NO: 12 or a sequence in which one or two amino acids in SEQ ID NO: 12 are substituted by other amino acids. In one embodiment, said ankyrin repeat module comprises the amino acid sequence of SEQ ID NO: 13 or a sequence in which one or two amino acids in SEQ ID NO: 13 are substituted by other amino acids. In one embodiment, said ankyrin repeat module comprises the amino acid sequence of SEQ ID NO: 14 or a sequence in which one or two amino acids in SEQ ID NO: 14 are substituted by other amino acids. In one embodiment, said ankyrin repeat module comprises the amino acid sequence of SEQ ID NO: 15 or a sequence in which one or two amino acids in SEQ ID NO: 15 are substituted by other amino acids. In one embodiment, said ankyrin repeat module comprises the amino acid sequence of SEQ ID NO: 16 or a sequence in which one or two amino acids in SEQ ID NO: 16 are substituted by other amino acids. In one embodiment, said ankyrin repeat module comprises the amino acid sequence of SEQ ID NO: 17 or a sequence in which one or two amino acids in SEQ ID NO: 17 are substituted by other amino acids. In one embodiment, said ankyrin repeat module comprises the amino acid sequence of SEQ ID NO: 18 or a sequence in which one or two amino acids in SEQ ID NO: 18 are substituted by other amino acids. In one embodiment, said ankyrin repeat module comprises the amino acid sequence of SEQ ID NO: 19 or a sequence in which one or two amino acids in SEQ ID NO: 19 are substituted by other amino acids. In one embodiment, said ankyrin repeat module comprises the amino acid sequence of SEQ ID NO: 20 or a sequence in which one or two amino acids in SEQ ID NO: 20 are substituted by other amino acids. In one embodiment, said ankyrin repeat module comprises the amino acid sequence of SEQ ID NO: 21 or a sequence in which one or two amino acids in SEQ ID NO: 21 are substituted by other amino acids. In one embodiment, said ankyrin repeat module comprises the amino acid sequence of SEQ ID NO: 9. In one embodiment, said ankyrin repeat module comprises the amino acid sequence of SEQ ID NO: 10. In one embodiment, said ankyrin repeat module comprises the amino acid sequence of SEQ ID NO: 11. In one embodiment, said ankyrin repeat module comprises the amino acid sequence of SEQ ID NO: 12. In one embodiment, said ankyrin repeat module comprises the amino acid sequence of SEQ ID NO: 13. In one embodiment, said ankyrin repeat module comprises the amino acid sequence of SEQ ID NO: 14. In one embodiment, said ankyrin repeat module comprises the amino acid sequence of SEQ ID NO: 15. In one embodiment, said ankyrin repeat module comprises the amino acid sequence of SEQ ID NO: 16. In one embodiment, said ankyrin repeat module comprises the amino acid sequence of SEQ ID NO: 17. In one embodiment, said ankyrin repeat module comprises the amino acid sequence of SEQ ID NO: 18. In one embodiment, said ankyrin repeat module comprises the amino acid sequence of SEQ ID NO: 19. In one embodiment, said ankyrin repeat module comprises the amino acid sequence of SEQ ID NO: 20. In one embodiment, said ankyrin repeat module comprises the amino acid sequence of SEQ ID NO: 21.

In one embodiment, said ankyrin repeat domain comprises a first ankyrin repeat module and a second ankyrin repeat module. In a preferred embodiment, said first ankyrin repeat module is located N-terminally of said second ankyrin repeat module within said ankyrin repeat domain. In one embodiment, said first and said second ankyrin repeat module each comprises an amino acid sequence selected from the group consisting of (1) SEQ ID NOs: 9 to 21 and (2) sequences in which up to 9, or up to 8, or up to 7, or up to 6, or up to 5, or up to 4, or up to 3, or up to 2, or up to 1 amino acids in any of SEQ ID NOs: 9 to 21 are substituted by other amino acids. Thus, in one embodiment, said ankyrin repeat domain comprises a first ankyrin repeat module having an amino acid sequence selected from the group consisting of (1) SEQ ID NOs: 9 to 21 and (2) sequences in which up to 9 amino acids in any of SEQ ID NOs: 9 to 21 are substituted by other amino acids and further comprises a second ankyrin repeat module having an amino acid sequence selected from the group consisting of (1) SEQ ID NOs: 9 to 21 and (2) sequences in which up to 9 amino acids in any of SEQ ID NOS: 9 to 21 are substituted by other amino acids.

In one particular embodiment, in such an ankyrin repeat domain said first ankyrin repeat module comprises an amino acid sequence selected from the group consisting of (1) SEQ ID NO: 9 and (2) sequences in which up to 9, or up to 8, or up to 7, or up to 6, or up to 5, or up to 4, or up to 3, or up to 2, or up to 1 amino acids in SEQ ID NO: 9 are substituted by other amino acids, and said second ankyrin repeat module comprises an amino acid sequence selected from the group consisting of (1) SEQ ID NO: 10 and (2) sequences in which up to 9, or up to 8, or up to 7, or up to 6, or up to 5, or up to 4, or up to 3, or up to 2, or up to 1 amino acids of SEQ ID NO: 10 are substituted by other amino acids.

Thus, in one embodiment, in such an ankyrin repeat domain said first ankyrin repeat module comprises an amino acid sequence selected from the group consisting of (1) SEQ ID NO: 9 and (2) sequences in which up to 9 amino acids in SEQ ID NO: 9 are substituted by other amino acids, and said second ankyrin repeat module comprises an amino acid sequence selected from the group consisting of (1) SEQ ID NO: 10 and (2) sequences in which up to 9 amino acids of SEQ ID NO: 10 are substituted by other amino acids. In one embodiment, in such an ankyrin repeat domain said first ankyrin repeat module comprises an amino acid sequence selected from the group consisting of (1) SEQ ID NO: 9 and (2) sequences in which up to 6 amino acids in SEQ ID NO: 9 are substituted by other amino acids, and said second ankyrin repeat module comprises an amino acid sequence selected from the group consisting of (1) SEQ ID NO: 10 and (2) sequences in which up to 6 amino acids of SEQ ID NO: 10 are substituted by other amino acids. In one embodiment, in such an ankyrin repeat domain said first ankyrin repeat module comprises an amino acid sequence selected from the group consisting of (1) SEQ ID NO: 9 and (2) sequences in which up to 5 amino acids in SEQ ID NO: 9 are substituted by other amino acids, and said second ankyrin repeat module comprises an amino acid sequence selected from the group consisting of (1) SEQ ID NO: 10 and (2) sequences in which up to 5 amino acids of SEQ ID NO: 10 are substituted by other amino acids. In one embodiment, in such an ankyrin repeat domain said first ankyrin repeat module comprises an amino acid sequence selected from the group consisting of (1) SEQ ID NO: 9 and (2) sequences in which up to 4 amino acids in SEQ ID NO: 9 are substituted by other amino acids, and said second ankyrin repeat module comprises an amino acid sequence selected from the group consisting of (1) SEQ ID NO: 10 and (2) sequences in which up to 4 amino acids of SEQ ID NO: 10 are substituted by other amino acids. In one embodiment, in such an ankyrin repeat domain said first ankyrin repeat module comprises an amino acid sequence selected from the group consisting of (1) SEQ ID NO: 9 and (2) sequences in which up to 3 amino acids in SEQ ID NO: 9 are substituted by other amino acids, and said second ankyrin repeat module comprises an amino acid sequence selected from the group consisting of (1) SEQ ID NO: 10 and (2) sequences in which up to 3 amino acids of SEQ ID NO: 10 are substituted by other amino acids. In one embodiment, in such an ankyrin repeat domain said first ankyrin repeat module comprises an amino acid sequence selected from the group consisting of (1) SEQ ID NO: 9 and (2) sequences in which up to 2 amino acids in SEQ ID NO: 9 are substituted by other amino acids, and said second ankyrin repeat module comprises an amino acid sequence selected from the group consisting of (1) SEQ ID NO: 10 and (2) sequences in which up to 2 amino acids of SEQ ID NO: 10 are substituted by other amino acids. In one embodiment, in such an ankyrin repeat domain said first ankyrin repeat module comprises an amino acid sequence selected from the group consisting of (1) SEQ ID NO: 9 and (2) sequences in which 1 amino acid in SEQ ID NO: 9 is substituted by another amino acid, and said second ankyrin repeat module comprises an amino acid sequence selected from the group consisting of (1) SEQ ID NO: 10 and (2) sequences in which 1 amino acid of SEQ ID NO: 10 is substituted by another amino acid. In one embodiment, in such an ankyrin repeat domain said first ankyrin repeat module comprises the amino acid sequence of SEQ ID NO: 9, and said second ankyrin repeat module comprises the amino acid sequence of SEQ ID NO: 10.

In a preferred embodiment, said first ankyrin repeat module comprises an amino acid sequence selected from the group consisting of (1) SEQ ID NO: 9 and (2) sequences in which up to 3, or up to 2, or up to 1 amino acids in SEQ ID NO: 9 are substituted by other amino acids, and said second ankyrin repeat module comprises an amino acid sequence selected from the group consisting of (1) SEQ ID NO: 10 and (2) sequences in which up to 3, or up to 2, or up to 1 amino acids of SEQ ID NO: 10 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 9 and said second ankyrin repeat module comprises the amino acid sequence of SEQ ID NO: 10, wherein said first ankyrin repeat module is located N-terminally of said second ankyrin repeat module within said ankyrin repeat domain.

In another particular embodiment, in such an ankyrin repeat domain said first ankyrin repeat module comprises an amino acid sequence selected from the group consisting of (1) SEQ ID NO: 11 and (2) sequences in which up to 9, or up to 8, or up to 7, or up to 6, or up to 5, or up to 4, or up to 3, or up to 2, or up to 1 amino acids in SEQ ID NO: 11 are substituted by other amino acids, and said second ankyrin repeat module comprises an amino acid sequence selected from the group consisting of (1) SEQ ID NO: 12 and (2) sequences in which up to 9, or up to 8, or up to 7, or up to 6, or up to 5, or up to 4, or up to 3, or up to 2, or up to 1 amino acids of SEQ ID NO: 12 are substituted by other amino acids.

Thus, in one embodiment, in such an ankyrin repeat domain said first ankyrin repeat module comprises an amino acid sequence selected from the group consisting of (1) SEQ ID NO: 11 and (2) sequences in which up to 9 amino acids in SEQ ID NO: 11 are substituted by other amino acids, and said second ankyrin repeat module comprises an amino acid sequence selected from the group consisting of (1) SEQ ID NO: 12 and (2) sequences in which up to 9 amino acids of SEQ ID NO: 12 are substituted by other amino acids. In one embodiment, in such an ankyrin repeat domain said first ankyrin repeat module comprises an amino acid sequence selected from the group consisting of (1) SEQ ID NO: 11 and (2) sequences in which up to 6 amino acids in SEQ ID NO: 11 are substituted by other amino acids, and said second ankyrin repeat module comprises an amino acid sequence selected from the group consisting of (1) SEQ ID NO: 12 and (2) sequences in which up to 6 amino acids of SEQ ID NO: 12 are substituted by other amino acids. In one embodiment, in such an ankyrin repeat domain said first ankyrin repeat module comprises an amino acid sequence selected from the group consisting of (1) SEQ ID NO: 11 and (2) sequences in which up to 5 amino acids in SEQ ID NO: 11 are substituted by other amino acids, and said second ankyrin repeat module comprises an amino acid sequence selected from the group consisting of (1) SEQ ID NO: 12 and (2) sequences in which up to 5 amino acids of SEQ ID NO: 12 are substituted by other amino acids. In one embodiment, in such an ankyrin repeat domain said first ankyrin repeat module comprises an amino acid sequence selected from the group consisting of (1) SEQ ID NO: 11 and (2) sequences in which up to 4 amino acids in SEQ ID NO: 11 are substituted by other amino acids, and said second ankyrin repeat module comprises an amino acid sequence selected from the group consisting of (1) SEQ ID NO: 12 and (2) sequences in which up to 4 amino acids of SEQ ID NO: 12 are substituted by other amino acids. In one embodiment, in such an ankyrin repeat domain said first ankyrin repeat module comprises an amino acid sequence selected from the group consisting of (1) SEQ ID NO: 11 and (2) sequences in which up to 3 amino acids in SEQ ID NO: 11 are substituted by other amino acids, and said second ankyrin repeat module comprises an amino acid sequence selected from the group consisting of (1) SEQ ID NO: 12 and (2) sequences in which up to 3 amino acids of SEQ ID NO: 12 are substituted by other amino acids. In one embodiment, in such an ankyrin repeat domain said first ankyrin repeat module comprises an amino acid sequence selected from the group consisting of (1) SEQ ID NO: 11 and (2) sequences in which up to 2 amino acids in SEQ ID NO: 11 are substituted by other amino acids, and said second ankyrin repeat module comprises an amino acid sequence selected from the group consisting of (1) SEQ ID NO: 12 and (2) sequences in which up to 2 amino acids of SEQ ID NO: 12 are substituted by other amino acids. In one embodiment, in such an ankyrin repeat domain said first ankyrin repeat module comprises an amino acid sequence selected from the group consisting of (1) SEQ ID NO: 11 and (2) sequences in which 1 amino acid in SEQ ID NO: 11 is substituted by another amino acid, and said second ankyrin repeat module comprises an amino acid sequence selected from the group consisting of (1) SEQ ID NO: 12 and (2) sequences in which 1 amino acid of SEQ ID NO: 12 is substituted by another amino acid. In one embodiment, in such an ankyrin repeat domain said first ankyrin repeat module comprises the amino acid sequence of SEQ ID NO: 11 and said second ankyrin repeat module comprises the amino acid sequence of SEQ ID NO: 12.

In one preferred embodiment, said first ankyrin repeat module comprises an amino acid sequence selected from the group consisting of (1) SEQ ID NO: 11 and (2) sequences in which up to 3, or up to 2, or up to 1 amino acids in SEQ ID NO: 11 are substituted by other amino acids, and said second ankyrin repeat module comprises an amino acid sequence selected from the group consisting of (1) SEQ ID NO: 12 and (2) sequences in which up to 3, or up to 2, or up to 1 amino acids of SEQ ID NO: 12 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 11 and said second ankyrin repeat module comprises the amino acid sequence of SEQ ID NO: 12, wherein said first ankyrin repeat module is located N-terminally of said second ankyrin repeat module within said ankyrin repeat domain. In another particular embodiment, in such an ankyrin repeat domain said first ankyrin repeat module comprises an amino acid sequence selected from the group consisting of (1) SEQ ID NO: 13 and (2) sequences in which up to 9, or up to 8, or up to 7, or up to 6, or up to 5, or up to 4, or up to 3, or up to 2, or up to 1 amino acids in SEQ ID NO: 13 are substituted by other amino acids, and said second ankyrin repeat module comprises an amino acid sequence selected from the group consisting of (1) SEQ ID NO: 14 and (2) sequences in which up to 9, or up to 8, or up to 7, or up to 6, or up to 5, or up to 4, or up to 3, or up to 2, or up to 1 amino acids of SEQ ID NO: 14 are substituted by other amino acids.

Thus, in one embodiment, in such an ankyrin repeat domain said first ankyrin repeat module comprises an amino acid sequence selected from the group consisting of (1) SEQ ID NO: 13 and (2) sequences in which up to 9 amino acids in SEQ ID NO: 13 are substituted by other amino acids, and said second ankyrin repeat module comprises an amino acid sequence selected from the group consisting of (1) SEQ ID NO: 14 and (2) sequences in which up to 9 amino acids of SEQ ID NO: 14 are substituted by other amino acids. In one embodiment, in such an ankyrin repeat domain said first ankyrin repeat module comprises an amino acid sequence selected from the group consisting of (1) SEQ ID NO: 13 and (2) sequences in which up to 6 amino acids in SEQ ID NO: 13 are substituted by other amino acids, and said second ankyrin repeat module comprises an amino acid sequence selected from the group consisting of (1) SEQ ID NO: 14 and (2) sequences in which up to 6 amino acids of SEQ ID NO: 14 are substituted by other amino acids. In one embodiment, in such an ankyrin repeat domain said first ankyrin repeat module comprises an amino acid sequence selected from the group consisting of (1) SEQ ID NO: 13 and (2) sequences in which up to 5 amino acids in SEQ ID NO: 13 are substituted by other amino acids, and said second ankyrin repeat module comprises an amino acid sequence selected from the group consisting of (1) SEQ ID NO: 14 and (2) sequences in which up to 5 amino acids of SEQ ID NO: 14 are substituted by other amino acids. In one embodiment, in such an ankyrin repeat domain said first ankyrin repeat module comprises an amino acid sequence selected from the group consisting of (1) SEQ ID NO: 13 and (2) sequences in which up to 4 amino acids in SEQ ID NO: 13 are substituted by other amino acids, and said second ankyrin repeat module comprises an amino acid sequence selected from the group consisting of (1) SEQ ID NO: 14 and (2) sequences in which up to 4 amino acids of SEQ ID NO: 14 are substituted by other amino acids. In one embodiment, in such an ankyrin repeat domain said first ankyrin repeat module comprises an amino acid sequence selected from the group consisting of (1) SEQ ID NO: 13 and (2) sequences in which up to 3 amino acids in SEQ ID NO: 13 are substituted by other amino acids, and said second ankyrin repeat module comprises an amino acid sequence selected from the group consisting of (1) SEQ ID NO: 14 and (2) sequences in which up to 3 amino acids of SEQ ID NO: 14 are substituted by other amino acids. In one embodiment, in such an ankyrin repeat domain said first ankyrin repeat module comprises an amino acid sequence selected from the group consisting of (1) SEQ ID NO: 13 and (2) sequences in which up to 2 amino acids in SEQ ID NO: 13 are substituted by other amino acids, and said second ankyrin repeat module comprises an amino acid sequence selected from the group consisting of (1) SEQ ID NO: 14 and (2) sequences in which up to 2 amino acids of SEQ ID NO: 14 are substituted by other amino acids. In one embodiment, in such an ankyrin repeat domain said first ankyrin repeat module comprises an amino acid sequence selected from the group consisting of (1) SEQ ID NO: 13 and (2) sequences in which 1 amino acid in SEQ ID NO: 13 is substituted by another amino acid, and said second ankyrin repeat module comprises an amino acid sequence selected from the group consisting of (1) SEQ ID NO: 14 and (2) sequences in which 1 amino acid of SEQ ID NO: 14 is substituted by another amino acid. In one embodiment, in such an ankyrin repeat domain said first ankyrin repeat module comprises the amino acid sequence of SEQ ID NO: 13, and said second ankyrin repeat module comprises the amino acid sequence of SEQ ID NO: 14.

In one preferred embodiment, said first ankyrin repeat module comprises an amino acid sequence selected from the group consisting of (1) SEQ ID NO: 13 and (2) sequences in which up to 3, or up to 2, or up to 1 amino acids in SEQ ID NO: 13 are substituted by other amino acids, and said second ankyrin repeat module comprises an amino acid sequence selected from the group consisting of (1) SEQ ID NO: 14 and (2) sequences in which up to 3, or up to 2, or up to 1 amino acids of SEQ ID NO: 14 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 13 and said second ankyrin repeat module comprises the amino acid sequence of SEQ ID NO: 14, wherein said first ankyrin repeat module is located N-terminally of said second ankyrin repeat module within said ankyrin repeat domain.

In another particular embodiment, in such an ankyrin repeat domain said first ankyrin repeat module comprises an amino acid sequence selected from the group consisting of (1) SEQ ID NO: 15 and (2) sequences in which up to 9, or up to 8, or up to 7, or up to 6, or up to 5, or up to 4, or up to 3, or up to 2, or up to 1 amino acids in SEQ ID NO: 15 are substituted by other amino acids, and said second ankyrin repeat module comprises an amino acid sequence selected from the group consisting of (1) SEQ ID NO: 16 and (2) sequences in which up to 9, or up to 8, or up to 7, or up to 6, or up to 5, or up to 4, or up to 3, or up to 2, or up to 1 amino acids of SEQ ID NO: 16 are substituted by other amino acids.

Thus, in one embodiment, in such an ankyrin repeat domain said first ankyrin repeat module comprises an amino acid sequence selected from the group consisting of (1) SEQ ID NO: 15 and (2) sequences in which up to 9 amino acids in SEQ ID NO: 15 are substituted by other amino acids, and said second ankyrin repeat module comprises an amino acid sequence selected from the group consisting of (1) SEQ ID NO: 16 and (2) sequences in which up to 9 amino acids of SEQ ID NO: 16 are substituted by other amino acids. In one embodiment, in such an ankyrin repeat domain said first ankyrin repeat module comprises an amino acid sequence selected from the group consisting of (1) SEQ ID NO: 15 and (2) sequences in which up to 6 amino acids in SEQ ID NO: 15 are substituted by other amino acids, and said second ankyrin repeat module comprises an amino acid sequence selected from the group consisting of (1) SEQ ID NO: 16 and (2) sequences in which up to 6 amino acids of SEQ ID NO: 16 are substituted by other amino acids. In one embodiment, in such an ankyrin repeat domain said first ankyrin repeat module comprises an amino acid sequence selected from the group consisting of (1) SEQ ID NO: 15 and (2) sequences in which up to 5 amino acids in SEQ ID NO: 15 are substituted by other amino acids, and said second ankyrin repeat module comprises an amino acid sequence selected from the group consisting of (1) SEQ ID NO: 16 and (2) sequences in which up to 5 amino acids of SEQ ID NO: 16 are substituted by other amino acids. In one embodiment, in such an ankyrin repeat domain said first ankyrin repeat module comprises an amino acid sequence selected from the group consisting of (1) SEQ ID NO: 15 and (2) sequences in which up to 4 amino acids in SEQ ID NO: 15 are substituted by other amino acids, and said second ankyrin repeat module comprises an amino acid sequence selected from the group consisting of (1) SEQ ID NO: 16 and (2) sequences in which up to 4 amino acids of SEQ ID NO: 16 are substituted by other amino acids. In one embodiment, in such an ankyrin repeat domain said first ankyrin repeat module comprises an amino acid sequence selected from the group consisting of (1) SEQ ID NO: 15 and (2) sequences in which up to 3 amino acids in SEQ ID NO: 15 are substituted by other amino acids, and said second ankyrin repeat module comprises an amino acid sequence selected from the group consisting of (1) SEQ ID NO: 16 and (2) sequences in which up to 3 amino acids of SEQ ID NO: 16 are substituted by other amino acids. In one embodiment, in such an ankyrin repeat domain said first ankyrin repeat module comprises an amino acid sequence selected from the group consisting of (1) SEQ ID NO: 15 and (2) sequences in which up to 2 amino acids in SEQ ID NO: 15 are substituted by other amino acids, and said second ankyrin repeat module comprises an amino acid sequence selected from the group consisting of (1) SEQ ID NO: 16 and (2) sequences in which up to 2 amino acids of SEQ ID NO: 16 are substituted by other amino acids. In one embodiment, in such an ankyrin repeat domain said first ankyrin repeat module comprises an amino acid sequence selected from the group consisting of (1) SEQ ID NO: 15 and (2) sequences in which 1 amino acid in SEQ ID NO: 15 is substituted by another amino acid, and said second ankyrin repeat module comprises an amino acid sequence selected from the group consisting of (1) SEQ ID NO: 16 and (2) sequences in which 1 amino acid of SEQ ID NO: 16 is substituted by another amino acid. In one embodiment, in such an ankyrin repeat domain said first ankyrin repeat module comprises the amino acid sequence of SEQ ID NO: 15, and said second ankyrin repeat module comprises the amino acid sequence of SEQ ID NO: 16.

In a preferred embodiment, said first ankyrin repeat module comprises an amino acid sequence selected from the group consisting of (1) SEQ ID NO: 15 and (2) sequences in which up to 3, or up to 2, or up to 1 amino acids in SEQ ID NO: 15 are substituted by other amino acids, and said second ankyrin repeat module comprises an amino acid sequence selected from the group consisting of (1) SEQ ID NO: 16 and (2) sequences in which up to 3, or up to 2, or up to 1 amino acids of SEQ ID NO: 16 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 15 and said second ankyrin repeat module comprises the amino acid sequence of SEQ ID NO:16, wherein said first ankyrin repeat module is located N-terminally of said second ankyrin repeat module within said ankyrin repeat domain.

In another particular embodiment, in such an ankyrin repeat domain said first ankyrin repeat module comprises an amino acid sequence selected from the group consisting of (1) SEQ ID NO: 17 and (2) sequences in which up to 9, or up to 8, or up to 7, or up to 6, or up to 5, or up to 4, or up to 3, or up to 2, or up to 1 amino acids in SEQ ID NO: 17 are substituted by other amino acids, and said second ankyrin repeat module comprises an amino acid sequence selected from the group consisting of (1) SEQ ID NO: 18 and (2) sequences in which up to 9, or up to 8, or up to 7, or up to 6, or up to 5, or up to 4, or up to 3, or up to 2, or up to 1 amino acids of SEQ ID NO: 18 are substituted by other amino acids.

Thus, in one embodiment, in such an ankyrin repeat domain said first ankyrin repeat module comprises an amino acid sequence selected from the group consisting of (1) SEQ ID NO: 17 and (2) sequences in which up to 9 amino acids in SEQ ID NO: 17 are substituted by other amino acids, and said second ankyrin repeat module comprises an amino acid sequence selected from the group consisting of (1) SEQ ID NO: 18 and (2) sequences in which up to 9 amino acids of SEQ ID NO: 18 are substituted by other amino acids. In one embodiment, in such an ankyrin repeat domain said first ankyrin repeat module comprises an amino acid sequence selected from the group consisting of (1) SEQ ID NO: 17 and (2) sequences in which up to 6 amino acids in SEQ ID NO: 17 are substituted by other amino acids, and said second ankyrin repeat module comprises an amino acid sequence selected from the group consisting of (1) SEQ ID NO: 18 and (2) sequences in which up to 6 amino acids of SEQ ID NO: 18 are substituted by other amino acids. In one embodiment, in such an ankyrin repeat domain said first ankyrin repeat module comprises an amino acid sequence selected from the group consisting of (1) SEQ ID NO: 17 and (2) sequences in which up to 5 amino acids in SEQ ID NO: 17 are substituted by other amino acids, and said second ankyrin repeat module comprises an amino acid sequence selected from the group consisting of (1) SEQ ID NO: 18 and (2) sequences in which up to 5 amino acids of SEQ ID NO: 18 are substituted by other amino acids. In one embodiment, in such an ankyrin repeat domain said first ankyrin repeat module comprises an amino acid sequence selected from the group consisting of (1) SEQ ID NO: 17 and (2) sequences in which up to 4 amino acids in SEQ ID NO: 17 are substituted by other amino acids, and said second ankyrin repeat module comprises an amino acid sequence selected from the group consisting of (1) SEQ ID NO: 18 and (2) sequences in which up to 4 amino acids of SEQ ID NO: 18 are substituted by other amino acids. In one embodiment, in such an ankyrin repeat domain said first ankyrin repeat module comprises an amino acid sequence selected from the group consisting of (1) SEQ ID NO: 17 and (2) sequences in which up to 3 amino acids in SEQ ID NO: 17 are substituted by other amino acids, and said second ankyrin repeat module comprises an amino acid sequence selected from the group consisting of (1) SEQ ID NO: 18 and (2) sequences in which up to 3 amino acids of SEQ ID NO: 18 are substituted by other amino acids. In one embodiment, in such an ankyrin repeat domain said first ankyrin repeat module comprises an amino acid sequence selected from the group consisting of (1) SEQ ID NO: 17 and (2) sequences in which up to 2 amino acids in SEQ ID NO: 17 are substituted by other amino acids, and said second ankyrin repeat module comprises an amino acid sequence selected from the group consisting of (1) SEQ ID NO: 18 and (2) sequences in which up to 2 amino acids of SEQ ID NO: 18 are substituted by other amino acids. In one embodiment, in such an ankyrin repeat domain said first ankyrin repeat module comprises an amino acid sequence selected from the group consisting of (1) SEQ ID NO: 17 and (2) sequences in which 1 amino acid in SEQ ID NO: 17 is substituted by another amino acid, and said second ankyrin repeat module comprises an amino acid sequence selected from the group consisting of (1) SEQ ID NO: 18 and (2) sequences in which 1 amino acid of SEQ ID NO: 18 is substituted by another amino acid. In one embodiment, in such an ankyrin repeat domain said first ankyrin repeat module comprises the amino acid sequence of SEQ ID NO: 17 and said second ankyrin repeat module comprises the amino acid sequence of SEQ ID NO: 18.

In a preferred embodiment, said first ankyrin repeat module comprises an amino acid sequence selected from the group consisting of (1) SEQ ID NO: 17 and (2) sequences in which up to 3, or up to 2, or up to 1 amino acids in SEQ ID NO: 17 are substituted by other amino acids, and said second ankyrin repeat module comprises an amino acid sequence selected from the group consisting of (1) SEQ ID NO: 18 and (2) sequences in which up to 3, or up to 2, or up to 1 amino acids of SEQ ID NO: 18 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 17 and said second ankyrin repeat module comprises the amino acid sequence of SEQ ID NO: 18, wherein said first ankyrin repeat module is located N-terminally of said second ankyrin repeat module within said ankyrin repeat domain.

In another particular embodiment, in such an ankyrin repeat domain said first ankyrin repeat module comprises an amino acid sequence selected from the group consisting of (1) SEQ ID NO: 13 and (2) sequences in which up to 9, or up to 8, or up to 7, or up to 6, or up to 5, or up to 4, or up to 3, or up to 2, or up to 1 amino acids in SEQ ID NO: 13 are substituted by other amino acids, and said second ankyrin repeat module comprises an amino acid sequence selected from the group consisting of (1) SEQ ID NO: 19 and (2) sequences in which up to 9, or up to 8, or up to 7, or up to 6, or up to 5, or up to 4, or up to 3, or up to 2, or up to 1 amino acids of SEQ ID NO: 19 are substituted by other amino acids.

Thus, in one embodiment, in such an ankyrin repeat domain said first ankyrin repeat module comprises an amino acid sequence selected from the group consisting of (1) SEQ ID NO: 13 and (2) sequences in which up to 9 amino acids in SEQ ID NO: 13 are substituted by other amino acids, and said second ankyrin repeat module comprises an amino acid sequence selected from the group consisting of (1) SEQ ID NO: 19 and (2) sequences in which up to 9 amino acids of SEQ ID NO: 19 are substituted by other amino acids. In one embodiment, in such an ankyrin repeat domain said first ankyrin repeat module comprises an amino acid sequence selected from the group consisting of (1) SEQ ID NO: 13 and (2) sequences in which up to 6 amino acids in SEQ ID NO: 13 are substituted by other amino acids, and said second ankyrin repeat module comprises an amino acid sequence selected from the group consisting of (1) SEQ ID NO: 19 and (2) sequences in which up to 6 amino acids of SEQ ID NO: 19 are substituted by other amino acids. In one embodiment, in such an ankyrin repeat domain said first ankyrin repeat module comprises an amino acid sequence selected from the group consisting of (1) SEQ ID NO: 13 and (2) sequences in which up to 5 amino acids in SEQ ID NO: 13 are substituted by other amino acids, and said second ankyrin repeat module comprises an amino acid sequence selected from the group consisting of (1) SEQ ID NO: 19 and (2) sequences in which up to 5 amino acids of SEQ ID NO: 19 are substituted by other amino acids. In one embodiment, in such an ankyrin repeat domain said first ankyrin repeat module comprises an amino acid sequence selected from the group consisting of (1) SEQ ID NO: 13 and (2) sequences in which up to 4 amino acids in SEQ ID NO: 13 are substituted by other amino acids, and said second ankyrin repeat module comprises an amino acid sequence selected from the group consisting of (1) SEQ ID NO: 19 and (2) sequences in which up to 4 amino acids of SEQ ID NO: 19 are substituted by other amino acids. In one embodiment, in such an ankyrin repeat domain said first ankyrin repeat module comprises an amino acid sequence selected from the group consisting of (1) SEQ ID NO: 13 and (2) sequences in which up to 3 amino acids in SEQ ID NO: 13 are substituted by other amino acids, and said second ankyrin repeat module comprises an amino acid sequence selected from the group consisting of (1) SEQ ID NO: 19 and (2) sequences in which up to 3 amino acids of SEQ ID NO: 19 are substituted by other amino acids. In one embodiment, in such an ankyrin repeat domain said first ankyrin repeat module comprises an amino acid sequence selected from the group consisting of (1) SEQ ID NO: 13 and (2) sequences in which up to 2 amino acids in SEQ ID NO: 19 are substituted by other amino acids, and said second ankyrin repeat module comprises an amino acid sequence selected from the group consisting of (1) SEQ ID NO: 13 and (2) sequences in which up to 2 amino acids of SEQ ID NO: 13 are substituted by other amino acids. In one embodiment, in such an ankyrin repeat domain said first ankyrin repeat module comprises an amino acid sequence selected from the group consisting of (1) SEQ ID NO: 13 and (2) sequences in which 1 amino acid in SEQ ID NO: 13 is substituted by another amino acid, and said second ankyrin repeat module comprises an amino acid sequence selected from the group consisting of (1) SEQ ID NO: 19 and (2) sequences in which 1 amino acid of SEQ ID NO: 19 is substituted by another amino acid. In one embodiment, in such an ankyrin repeat domain said first ankyrin repeat module comprises the amino acid sequence of SEQ ID NO: 13, and said second ankyrin repeat module comprises the amino acid sequence of SEQ ID NO: 19.

In a preferred embodiment, said first ankyrin repeat module comprises an amino acid sequence selected from the group consisting of (1) SEQ ID NO: 13 and (2) sequences in which up to 3, or up to 2, or up to 1 amino acids in SEQ ID NO: 13 are substituted by other amino acids, and said second ankyrin repeat module comprises an amino acid sequence selected from the group consisting of (1) SEQ ID NO: 19 and (2) sequences in which up to 3, or up to 2, or up to 1 amino acids of SEQ ID NO: 19 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 13 and said second ankyrin repeat module comprises the amino acid sequence of SEQ ID NO: 19, wherein said first ankyrin repeat module is located N-terminally of said second ankyrin repeat module within said ankyrin repeat domain.

In another particular embodiment, in such an ankyrin repeat domain said first ankyrin repeat module comprises an amino acid sequence selected from the group consisting of (1) SEQ ID NO: 17 and (2) sequences in which up to 9, or up to 8, or up to 7, or up to 6, or up to 5, or up to 4, or up to 3, or up to 2, or up to 1 amino acids in SEQ ID NO: 17 are substituted by other amino acids, and said second ankyrin repeat module comprises an amino acid sequence selected from the group consisting of (1) SEQ ID NO: 20 and (2) sequences in which up to 9, or up to 8, or up to 7, or up to 6, or up to 5, or up to 4, or up to 3, or up to 2, or up to 1 amino acids of SEQ ID NO: 20 are substituted by other amino acids.

Thus, in one embodiment, in such an ankyrin repeat domain said first ankyrin repeat module comprises an amino acid sequence selected from the group consisting of (1) SEQ ID NO: 17 and (2) sequences in which up to 9 amino acids in SEQ ID NO: 17 are substituted by other amino acids, and said second ankyrin repeat module comprises an amino acid sequence selected from the group consisting of (1) SEQ ID NO: 20 and (2) sequences in which up to 9 amino acids of SEQ ID NO: 20 are substituted by other amino acids. In one embodiment, in such an ankyrin repeat domain said first ankyrin repeat module comprises an amino acid sequence selected from the group consisting of (1) SEQ ID NO: 17 and (2) sequences in which up to 6 amino acids in SEQ ID NO: 17 are substituted by other amino acids, and said second ankyrin repeat module comprises an amino acid sequence selected from the group consisting of (1) SEQ ID NO: 20 and (2) sequences in which up to 6 amino acids of SEQ ID NO: 20 are substituted by other amino acids. In one embodiment, in such an ankyrin repeat domain said first ankyrin repeat module comprises an amino acid sequence selected from the group consisting of (1) SEQ ID NO: 17 and (2) sequences in which up to 5 amino acids in SEQ ID NO: 17 are substituted by other amino acids, and said second ankyrin repeat module comprises an amino acid sequence selected from the group consisting of (1) SEQ ID NO: 20 and (2) sequences in which up to 5 amino acids of SEQ ID NO: 20 are substituted by other amino acids. In one embodiment, in such an ankyrin repeat domain said first ankyrin repeat module comprises an amino acid sequence selected from the group consisting of (1) SEQ ID NO:17 and (2) sequences in which up to 4 amino acids in SEQ ID NO: 17 are substituted by other amino acids, and said second ankyrin repeat module comprises an amino acid sequence selected from the group consisting of (1) SEQ ID NO: 20 and (2) sequences in which up to 4 amino acids of SEQ ID NO: 20 are substituted by other amino acids. In one embodiment, in such an ankyrin repeat domain said first ankyrin repeat module comprises an amino acid sequence selected from the group consisting of (1) SEQ ID NO: 17 and (2) sequences in which up to 3 amino acids in SEQ ID NO: 17 are substituted by other amino acids, and said second ankyrin repeat module comprises an amino acid sequence selected from the group consisting of (1) SEQ ID NO: 20 and (2) sequences in which up to 3 amino acids of SEQ ID NO: 20 are substituted by other amino acids. In one embodiment, in such an ankyrin repeat domain said first ankyrin repeat module comprises an amino acid sequence selected from the group consisting of (1) SEQ ID NO: 17 and (2) sequences in which up to 2 amino acids in SEQ ID NO: 17 are substituted by other amino acids, and said second ankyrin repeat module comprises an amino acid sequence selected from the group consisting of (1) SEQ ID NO: 20 and (2) sequences in which up to 2 amino acids of SEQ ID NO: 20 are substituted by other amino acids. In one embodiment, in such an ankyrin repeat domain said first ankyrin repeat module comprises an amino acid sequence selected from the group consisting of (1) SEQ ID NO: 17 and (2) sequences in which 1 amino acid in SEQ ID NO: 17 is substituted by another amino acid, and said second ankyrin repeat module comprises an amino acid sequence selected from the group consisting of (1) SEQ ID NO: 20 and (2) sequences in which 1 amino acid of SEQ ID NO: 20 is substituted by another amino acid. In one embodiment, in such an ankyrin repeat domain said first ankyrin repeat module comprises the amino acid sequence of SEQ ID NO: 17 and said second ankyrin repeat module comprises the amino acid sequence of SEQ ID NO: 20.

In a preferred embodiment, said first ankyrin repeat module comprises an amino acid sequence selected from the group consisting of (1) SEQ ID NO: 17 and (2) sequences in which up to 3, or up to 2, or up to 1 amino acids in SEQ ID NO: 17 are substituted by other amino acids, and said second ankyrin repeat module comprises an amino acid sequence selected from the group consisting of (1) SEQ ID NO: 20 and (2) sequences in which up to 3, or up to 2, or up to 1 amino acids of SEQ ID NO: 20 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 17 and said second ankyrin repeat module comprises the amino acid sequence of SEQ ID NO: 20, wherein said first ankyrin repeat module is located N-terminally of said second ankyrin repeat module within said ankyrin repeat domain.

In another particular embodiment, in such an ankyrin repeat domain said first ankyrin repeat module comprises an amino acid sequence selected from the group consisting of (1) SEQ ID NO: 17 and (2) sequences in which up to 9, or up to 8, or up to 7, or up to 6, or up to 5, or up to 4, or up to 3, or up to 2, or up to 1 amino acids in SEQ ID NO: 17 are substituted by other amino acids, and said second ankyrin repeat module comprises an amino acid sequence selected from the group consisting of (1) SEQ ID NO: 21 and (2) sequences in which up to 9, or up to 8, or up to 7, or up to 6, or up to 5, or up to 4, or up to 3, or up to 2, or up to 1 amino acids of SEQ ID NO: 21 are substituted by other amino acids.

Thus, in one embodiment, in such an ankyrin repeat domain said first ankyrin repeat module comprises an amino acid sequence selected from the group consisting of (1) SEQ ID NO: 17 and (2) sequences in which up to 9 amino acids in SEQ ID NO: 17 are substituted by other amino acids, and said second ankyrin repeat module comprises an amino acid sequence selected from the group consisting of (1) SEQ ID NO: 21 and (2) sequences in which up to 9 amino acids of SEQ ID NO: 21 are substituted by other amino acids. In one embodiment, in such an ankyrin repeat domain said first ankyrin repeat module comprises an amino acid sequence selected from the group consisting of (1) SEQ ID NO: 17 and (2) sequences in which up to 6 amino acids in SEQ ID NO: 17 are substituted by other amino acids, and said second ankyrin repeat module comprises an amino acid sequence selected from the group consisting of (1) SEQ ID NO: 21 and (2) sequences in which up to 6 amino acids of SEQ ID NO: 21 are substituted by other amino acids. In one embodiment, in such an ankyrin repeat domain said first ankyrin repeat module comprises an amino acid sequence selected from the group consisting of (1) SEQ ID NO: 17 and (2) sequences in which up to 5 amino acids in SEQ ID NO: 17 are substituted by other amino acids, and said second ankyrin repeat module comprises an amino acid sequence selected from the group consisting of (1) SEQ ID NO: 21 and (2) sequences in which up to 5 amino acids of SEQ ID NO: 21 are substituted by other amino acids. In one embodiment, in such an ankyrin repeat domain said first ankyrin repeat module comprises an amino acid sequence selected from the group consisting of (1) SEQ ID NO: 17 and (2) sequences in which up to 4 amino acids in SEQ ID NO: 17 are substituted by other amino acids, and said second ankyrin repeat module comprises an amino acid sequence selected from the group consisting of (1) SEQ ID NO: 21 and (2) sequences in which up to 4 amino acids of SEQ ID NO: 21 are substituted by other amino acids. In one embodiment, in such an ankyrin repeat domain said first ankyrin repeat module comprises an amino acid sequence selected from the group consisting of (1) SEQ ID NO: 17 and (2) sequences in which up to 3 amino acids in SEQ ID NO: 17 are substituted by other amino acids, and said second ankyrin repeat module comprises an amino acid sequence selected from the group consisting of (1) SEQ ID NO: 21 and (2) sequences in which up to 3 amino acids of SEQ ID NO: 21 are substituted by other amino acids. In one embodiment, in such an ankyrin repeat domain said first ankyrin repeat module comprises an amino acid sequence selected from the group consisting of (1) SEQ ID NO: 17 and (2) sequences in which up to 2 amino acids in SEQ ID NO: 17 are substituted by other amino acids, and said second ankyrin repeat module comprises an amino acid sequence selected from the group consisting of (1) SEQ ID NO: 21 and (2) sequences in which up to 2 amino acids of SEQ ID NO: 21 are substituted by other amino acids. In one embodiment, in such an ankyrin repeat domain said first ankyrin repeat module comprises an amino acid sequence selected from the group consisting of (1) SEQ ID NO: 17 and (2) sequences in which 1 amino acid in SEQ ID NO: 17 is substituted by another amino acid, and said second ankyrin repeat module comprises an amino acid sequence selected from the group consisting of (1) SEQ ID NO: 21 and (2) sequences in which 1 amino acid of SEQ ID NO: 21 is substituted by another amino acid. In one embodiment, in such an ankyrin repeat domain said first ankyrin repeat module comprises the amino acid sequence of SEQ ID NO: 17 and said second ankyrin repeat module comprises the amino acid sequence of SEQ ID NO: 21.

In a preferred embodiment, said first ankyrin repeat module comprises an amino acid sequence selected from the group consisting of (1) SEQ ID NO: 17 and (2) sequences in which up to 3, or up to 2, or up to 1 amino acids in SEQ ID NO: 17 are substituted by other amino acids, and said second ankyrin repeat module comprises an amino acid sequence selected from the group consisting of (1) SEQ ID NO: 21 and (2) sequences in which up to 3, or up to 2, or up to 1 amino acids of SEQ ID NO: 21 are substituted by other amino acids, wherein said first ankyrin repeat module is located N-terminally of said second ankyrin repeat module within said ankyrin repeat domain. Further, in a more preferred embodiment, said first ankyrin repeat module comprises the amino acid sequence of SEQ ID NO: 17 and said second ankyrin repeat module comprises the amino acid sequence of SEQ ID NO: 21, wherein said first ankyrin repeat module is located N-terminally of said second ankyrin repeat module within said ankyrin repeat domain.

In one preferred embodiment, all of said amino acid substitutions of said ankyrin repeat module(s) as described and referred to herein occur in framework positions of said ankyrin repeat module(s), wherein typically the overall structure of the module(s) is not affected by the substitutions. Such an embodiment of substitution in framework positions shall apply to all embodiments irrespective of whether such substitution is explicitly described.

In one preferred embodiment, all of said amino acid substitutions of said ankyrin repeat module(s) as described and referred to herein occur in framework positions and in positions other than positions 3, 4, 6, 14 and 15, preferably other than positions 2, 3, 4, 5, 6, 11, 14 and 15, of said ankyrin repeat module(s) of SEQ ID NOs: 9 to 21, wherein typically the overall structure of the module(s) is not affected by the substitutions.

In another aspect, the invention relates to a recombinant binding protein comprising an ankyrin repeat domain, wherein said ankyrin repeat domain has binding specificity for CD47, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1 to 8, and wherein A at the second last position of SEQ ID NOs: 1 to 8 is optionally substituted by L, and/or A at the last position of SEQ ID NOS: 1 to 8 is optionally substituted by N.

Thus, in one embodiment, said ankyrin repeat domain comprises an amino acid sequence with at least 80% amino acid sequence identity with any one of SEQ ID NOs: 1 to 8. In one embodiment, said ankyrin repeat domain comprises an amino acid sequence with at least 90% amino acid sequence identity with any one of SEQ ID NOs: 1 to 8. In another embodiment, said ankyrin repeat domain comprises an amino acid sequence with at least 93% amino acid sequence identity with any one of SEQ ID NOs: 1 to 8; and in a further embodiment, said ankyrin repeat domain comprises an amino acid sequence with at least 95% amino acid sequence identity with any one of SEQ ID NOS: 1 to 8. In one embodiment, said ankyrin repeat domain comprises an amino acid sequence with at least 98% amino acid sequence identity with any one of SEQ ID NOS: 1 to 8; and in one embodiment, said ankyrin repeat domain comprises the amino acid sequence of any one of SEQ ID NOs: 1 to 8. Thus, in one embodiment, said recombinant binding protein comprises an ankyrin repeat domain with binding specificity for CD47, wherein said ankyrin repeat domain comprises an amino acid sequence selected from SEQ ID NOS: 1 to 8, and wherein A at the second last position of SEQ ID NOS: 1 to 8 is optionally substituted by L, and/or A at the last position of SEQ ID NOS: 1 to 8 is optionally substituted by N.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 1, and wherein A at the second last position of SEQ ID NO: 1 is optionally substituted by L, and/or A at the last position of SEQ ID NO: 1 is optionally substituted by N. Thus, in one embodiment, said ankyrin repeat domain comprises an amino acid sequence with at least 80% amino acid sequence identity with SEQ ID NO: 1. In one embodiment, said ankyrin repeat domain comprises an amino acid sequence with at least 90% amino acid sequence identity with SEQ ID NO: 1. In another embodiment, said ankyrin repeat domain comprises an amino acid sequence with at least 93% amino acid sequence identity with SEQ ID NO: 1; and in a further embodiment, said ankyrin repeat domain comprises an amino acid sequence with at least 95% amino acid sequence identity with SEQ ID NO: 1. In one embodiment, said ankyrin repeat domain comprises an amino acid sequence with at least 98% amino acid sequence identity with SEQ ID NO: 1; and in one embodiment, said ankyrin repeat domain comprises the amino acid sequence of SEQ ID NO: 1. Thus, in one embodiment, said recombinant binding protein comprises an ankyrin repeat domain having binding specificity for CD47, wherein said ankyrin repeat domain comprises the amino acid sequence of SEQ ID NO: 1.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 2, and wherein A at the second last position of SEQ ID NO: 2 is optionally substituted by L, and/or A at the last position of SEQ ID NO: 2 is optionally substituted by N. Thus, in one embodiment, said ankyrin repeat domain comprises an amino acid sequence with at least 80% amino acid sequence identity with SEQ ID NO: 2. In one embodiment, said ankyrin repeat domain comprises an amino acid sequence with at least 90% amino acid sequence identity with SEQ ID NO: 2. In another embodiment, said ankyrin repeat domain comprises an amino acid sequence with at least 93% amino acid sequence identity with SEQ ID NO: 2; and in a further embodiment, said ankyrin repeat domain comprises an amino acid sequence with at least 95% amino acid sequence identity with SEQ ID NO: 2. In one embodiment, said ankyrin repeat domain comprises an amino acid sequence with at least 98% amino acid sequence identity with SEQ ID NO: 2; and in one embodiment, said ankyrin repeat domain comprises the amino acid sequence of SEQ ID NO: 2. Thus, in one embodiment, said recombinant binding protein comprises an ankyrin repeat domain having binding specificity for CD47, wherein said ankyrin repeat domain comprises the amino acid sequence of SEQ ID NO: 2.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 3, and wherein A at the second last position of SEQ ID NO: 3 is optionally substituted by L, and/or A at the last position of SEQ ID NO: 3 is optionally substituted by N. Thus, in one embodiment, said ankyrin repeat domain comprises an amino acid sequence with at least 80% amino acid sequence identity with SEQ ID NO: 3. In one embodiment, said ankyrin repeat domain comprises an amino acid sequence with at least 90% amino acid sequence identity with SEQ ID NO: 3. In another embodiment, said ankyrin repeat domain comprises an amino acid sequence with at least 93% amino acid sequence identity with SEQ ID NO: 3; and in a further embodiment, said ankyrin repeat domain comprises an amino acid sequence with at least 95% amino acid sequence identity with SEQ ID NO: 3. In one embodiment, said ankyrin repeat domain comprises an amino acid sequence with at least 98% amino acid sequence identity with SEQ ID NO: 3; and in one embodiment, said ankyrin repeat domain comprises the amino acid sequence of SEQ ID NO: 3. Thus, in one embodiment, said recombinant binding protein comprises an ankyrin repeat domain having binding specificity for CD47, wherein said ankyrin repeat domain comprises the amino acid sequence of SEQ ID NO: 3.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 4, and wherein A at the second last position of SEQ ID NO: 4 is optionally substituted by L, and/or A at the last position of SEQ ID NO: 4 is optionally substituted by N. Thus, in one embodiment, said ankyrin repeat domain comprises an amino acid sequence with at least 80% amino acid sequence identity with SEQ ID NO: 4. In one embodiment, said ankyrin repeat domain comprises an amino acid sequence with at least 90% amino acid sequence identity with SEQ ID NO: 4. In another embodiment, said ankyrin repeat domain comprises an amino acid sequence with at least 93% amino acid sequence identity with SEQ ID NO: 4; and in a further embodiment, said ankyrin repeat domain comprises an amino acid sequence with at least 95% amino acid sequence identity with SEQ ID NO: 4. In one embodiment, said ankyrin repeat domain comprises an amino acid sequence with at least 98% amino acid sequence identity with SEQ ID NO: 4; and in one embodiment, said ankyrin repeat domain comprises the amino acid sequence of SEQ ID NO: 4. Thus, in one embodiment, said recombinant binding protein comprises an ankyrin repeat domain having binding specificity for CD47, wherein said ankyrin repeat domain comprises the amino acid sequence of SEQ ID NO: 4.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 5, and wherein A at the second last position of SEQ ID NO: 5 is optionally substituted by L, and/or A at the last position of SEQ ID NO: 5 is optionally substituted by N. Thus, in one embodiment, said ankyrin repeat domain comprises an amino acid sequence with at least 80% amino acid sequence identity with SEQ ID NO: 5. In one embodiment, said ankyrin repeat domain comprises an amino acid sequence with at least 90% amino acid sequence identity with SEQ ID NO: 5. In another embodiment, said ankyrin repeat domain comprises an amino acid sequence with at least 93% amino acid sequence identity with SEQ ID NO: 5; and in a further embodiment, said ankyrin repeat domain comprises an amino acid sequence with at least 95% amino acid sequence identity with SEQ ID NO: 5. In one embodiment, said ankyrin repeat domain comprises an amino acid sequence with at least 98% amino acid sequence identity with SEQ ID NO: 5; and in one embodiment, said ankyrin repeat domain comprises the amino acid sequence of SEQ ID NO: 5. Thus, in one embodiment, said recombinant binding protein comprises an ankyrin repeat domain having binding specificity for CD47, wherein said ankyrin repeat domain comprises the amino acid sequence of SEQ ID NO: 5.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 6, and wherein A at the second last position of SEQ ID NO: 6 is optionally substituted by L, and/or A at the last position of SEQ ID NO: 6 is optionally substituted by N. Thus, in one embodiment, said ankyrin repeat domain comprises an amino acid sequence with at least 80% amino acid sequence identity with SEQ ID NO: 6. In one embodiment, said ankyrin repeat domain comprises an amino acid sequence with at least 90% amino acid sequence identity with SEQ ID NO: 6. In another embodiment, said ankyrin repeat domain comprises an amino acid sequence with at least 93% amino acid sequence identity with SEQ ID NO: 6; and in a further embodiment, said ankyrin repeat domain comprises an amino acid sequence with at least 95% amino acid sequence identity with SEQ ID NO: 6. In one embodiment, said ankyrin repeat domain comprises an amino acid sequence with at least 98% amino acid sequence identity with SEQ ID NO: 6; and in one embodiment, said ankyrin repeat domain comprises the amino acid sequence of SEQ ID NO: 6. Thus, in one embodiment, said recombinant binding protein comprises an ankyrin repeat domain having binding specificity for CD47, wherein said ankyrin repeat domain comprises the amino acid sequence of SEQ ID NO: 6.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 7, and wherein A at the second last position of SEQ ID NO: 7 is optionally substituted by L, and/or A at the last position of SEQ ID NO: 7 is optionally substituted by N. Thus, in one embodiment, said ankyrin repeat domain comprises an amino acid sequence with at least 80% amino acid sequence identity with SEQ ID NO: 7. In one embodiment, said ankyrin repeat domain comprises an amino acid sequence with at least 90% amino acid sequence identity with SEQ ID NO: 7. In another embodiment, said ankyrin repeat domain comprises an amino acid sequence with at least 93% amino acid sequence identity with SEQ ID NO: 7; and in a further embodiment, said ankyrin repeat domain comprises an amino acid sequence with at least 95% amino acid sequence identity with SEQ ID NO: 7. In one embodiment, said ankyrin repeat domain comprises an amino acid sequence with at least 98% amino acid sequence identity with SEQ ID NO: 7; and in one embodiment, said ankyrin repeat domain comprises the amino acid sequence of SEQ ID NO: 7. Thus, in one embodiment, said recombinant binding protein comprises an ankyrin repeat domain having binding specificity for CD47, wherein said ankyrin repeat domain comprises the amino acid sequence of SEQ ID NO: 7.

In one embodiment, said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 8, and wherein A at the second last position of SEQ ID NO: 8 is optionally substituted by L, and/or A at the last position of SEQ ID NO: 8 is optionally substituted by N. Thus, in one embodiment, said ankyrin repeat domain comprises an amino acid sequence with at least 80% amino acid sequence identity with SEQ ID NO: 8. In one embodiment, said ankyrin repeat domain comprises an amino acid sequence with at least 90% amino acid sequence identity with SEQ ID NO: 8. In another embodiment, said ankyrin repeat domain comprises an amino acid sequence with at least 93% amino acid sequence identity with SEQ ID NO: 8; and in a further embodiment, said ankyrin repeat domain comprises an amino acid sequence with at least 95% amino acid sequence identity with SEQ ID NO: 8. In one embodiment, said ankyrin repeat domain comprises an amino acid sequence with at least 98% amino acid sequence identity with SEQ ID NO: 8; and in one embodiment, said ankyrin repeat domain comprises the amino acid sequence of SEQ ID NO: 8. Thus, in one embodiment, said recombinant binding protein comprises an ankyrin repeat domain having binding specificity for CD47, wherein said ankyrin repeat domain comprises the amino acid sequence of SEQ ID NO: 8.

In one embodiment, said recombinant binding protein comprises an ankyrin repeat domain with binding specificity for CD47, wherein the potential interaction residues in said ankyrin repeat domain are identical to the corresponding positions in any one of the ankyrin repeat domains of SEQ ID NOs: 1 to 8.

In one embodiment, the invention relates to a recombinant binding protein comprising an ankyrin repeat domain with binding specificity for CD47, wherein said recombinant binding protein binds to human CD47 in PBS with a dissociation constant (K_{D}) of or below about 10⁻⁷M, or of or below about 10⁻⁸M, or of or below about 10⁻⁹M. Thus, in one embodiment, said recombinant binding protein binds human CD47 in PBS with a dissociation constant (K_{D}) of or below about 10⁻⁷M. In another embodiment, said recombinant binding protein binds human CD47 in PBS with a dissociation constant (K_{D}) of or below about 10⁻⁸M. In another embodiment, said recombinant binding protein binds human CD47 in PBS with a dissociation constant (K_{D}) of or below about 10⁻⁹M.

In one embodiment, said recombinant binding protein comprises an ankyrin repeat domain with binding specificity for CD47, wherein said recombinant binding protein binds human CD47 in PBS with a dissociation constant (K_{D}) of or below 10⁻⁷M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOS: 1 to 8, wherein A at the second last position of SEQ ID NOs: 1 to 8 is optionally substituted by L, and/or A at the last position of SEQ ID NOS: 1 to 8 is optionally substituted by N. In another embodiment, said recombinant binding protein comprises an ankyrin repeat domain with binding specificity for CD47, wherein said binding protein binds human CD47 in PBS with a dissociation constant (K_{D}) of or below 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with any one of SEQ ID NOs: 1 to 8, wherein A at the second last position of SEQ ID NOs: 1 to 8 is optionally substituted by L, and/or A at the last position of SEQ ID NOs: 1 to 8 is optionally substituted by N. Thus, in one embodiment, said recombinant binding protein binds human CD47 in PBS with a dissociation constant (K_{D}) below 10⁻⁷M, and said ankyrin repeat domain comprises an amino acid sequence with at least 80% amino acid sequence identity with any one of SEQ ID NOs: 1 to 8. In one embodiment, said recombinant binding protein binds human CD47 in PBS with a dissociation constant (K_{D}) below 10⁻⁷M, and said ankyrin repeat domain comprises an amino acid sequence with at least 90% amino acid sequence identity with any one of SEQ ID NOS: 1 to 8. In another embodiment, said recombinant binding protein binds human CD47 in PBS with a dissociation constant (K_{D}) below 10⁻⁷M, and said ankyrin repeat domain comprises an amino acid sequence with at least 93% amino acid sequence identity with any one of SEQ ID NOs: 1 to 8; and in a further embodiment, said binding protein binds human CD47 in PBS with a dissociation constant (K_{D}) below 10⁻⁷M, and said ankyrin repeat domain comprises an amino acid sequence with at least 95% amino acid sequence identity with any one of SEQ ID NOs: 1 to 8. In one embodiment, said recombinant binding protein binds human CD47 in PBS with a dissociation constant (K_{D}) below 10⁻⁷M, and said ankyrin repeat domain comprises an amino acid sequence with at least 98% amino acid sequence identity with any one of SEQ ID NOs: 1 to 8; and in one embodiment, said recombinant binding protein binds human CD47 in PBS with a dissociation constant (K_{D}) below 10⁻⁷M, and said ankyrin repeat domain comprises the amino acid sequence of any one of SEQ ID NOs: 1 to 8. Thus, in one embodiment, said recombinant binding protein comprises an ankyrin repeat domain with binding specificity for CD47, wherein said recombinant binding protein binds human CD47 in PBS with a dissociation constant (K_{D}) of or below about 10⁻⁷M, and wherein said ankyrin repeat domain comprises the amino acid sequence of any one of SEQ ID NOs: 1 to 8, wherein A at the second last position of SEQ ID NOs: 1 to 8 is optionally substituted by L, and/or A at the last position of SEQ ID NOs: 1 to 8 is optionally substituted by N.

In one embodiment, said recombinant binding protein comprises an ankyrin repeat domain having binding specificity for CD47, wherein said binding protein binds human CD47 in PBS with a dissociation constant (K_{D}) of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 1, and wherein A at the second last position of SEQ ID NO: 1 is optionally substituted by L, and/or A at the last position of SEQ ID NO: 1 is optionally substituted by N. Thus, in one embodiment, said recombinant binding protein binds human CD47 in PBS with a dissociation constant (K_{D}) of or below about 10⁻⁸M, and said ankyrin repeat domain comprises an amino acid sequence with at least 80% amino acid sequence identity with SEQ ID NO: 1. In one embodiment, said binding protein binds human CD47 in PBS with a dissociation constant (K_{D}) below 10 8M, and said ankyrin repeat domain comprises an amino acid sequence with at least 90% amino acid sequence identity with SEQ ID NO: 1. In another embodiment, said recombinant binding protein binds human CD47 in PBS with a dissociation constant (K_{D}) below 10⁻⁸M, and said ankyrin repeat domain comprises an amino acid sequence with at least 93% amino acid sequence identity with SEQ ID NO: 1; and in a further embodiment, said recombinant binding protein binds human CD47 in PBS with a dissociation constant (K_{D}) below 10⁻⁸M, and said ankyrin repeat domain comprises an amino acid sequence with at least 95% amino acid sequence identity with SEQ ID NO: 1. In one embodiment, said recombinant binding protein binds human CD47 in PBS with a dissociation constant (K_{D}) below 10⁻⁸M, and said ankyrin repeat domain comprises an amino acid sequence with at least 98% amino acid sequence identity with SEQ ID NO: 1; and in one embodiment, said recombinant binding protein binds human CD47 in PBS with a dissociation constant (K_{D}) below 10⁻⁸M, and said ankyrin repeat domain comprises the amino acid sequence of SEQ ID NO: 1. Thus, in one embodiment, said recombinant binding protein comprises an ankyrin repeat domain with binding specificity for CD47, wherein said recombinant binding protein binds human CD47 in PBS with a dissociation constant (K_{D}) of or below about 10⁻⁸M, and wherein said ankyrin repeat domain comprises the amino acid sequence of SEQ ID NO: 1, and wherein A at the second last position of SEQ ID NO: 1 is optionally substituted by L, and/or A at the last position of SEQ ID NO: 1 is optionally substituted by N.

In one embodiment, said recombinant binding protein comprises an ankyrin repeat domain having binding specificity for CD47, wherein said binding protein binds human CD47 in PBS with a dissociation constant (K_{D}) of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 2, and wherein A at the second last position of SEQ ID NO: 2 is optionally substituted by L, and/or A at the last position of SEQ ID NO: 2 is optionally substituted by N. Thus, in one embodiment, said recombinant binding protein binds human CD47 in PBS with a dissociation constant (K_{D}) of or below about 10⁻⁸M, and said ankyrin repeat domain comprises an amino acid sequence with at least 80% amino acid sequence identity with SEQ ID NO: 2. In one embodiment, said recombinant binding protein binds human CD47 in PBS with a dissociation constant (K_{D}) below 10⁻⁸M, and said ankyrin repeat domain comprises an amino acid sequence with at least 90% amino acid sequence identity with SEQ ID NO: 2. In another embodiment, said recombinant binding protein binds human CD47 in PBS with a dissociation constant (K_{D}) below 10⁻⁸M, and said ankyrin repeat domain comprises an amino acid sequence with at least 93% amino acid sequence identity with SEQ ID NO: 2; and in a further embodiment, said recombinant binding protein binds human CD47 in PBS with a dissociation constant (K_{D}) below 10⁻⁸M, and said ankyrin repeat domain comprises an amino acid sequence with at least 95% amino acid sequence identity with SEQ ID NO: 2. In one embodiment, said recombinant binding protein binds human CD47 in PBS with a dissociation constant (K_{D}) below 10⁻⁸M, and said ankyrin repeat domain comprises an amino acid sequence with at least 98% amino acid sequence identity with SEQ ID NO: 2; and in one embodiment, said recombinant binding protein binds human CD47 in PBS with a dissociation constant (K_{D}) below 10⁻⁸M, and said ankyrin repeat domain comprises the amino acid sequence of SEQ ID NO: 2. Thus, in one embodiment, said recombinant binding protein comprises an ankyrin repeat domain with binding specificity for CD47, wherein said recombinant binding protein binds human CD47 in PBS with a dissociation constant (K_{D}) of or below about 10⁻⁸M, and wherein said ankyrin repeat domain comprises the amino acid sequence of SEQ ID NO: 2, and wherein A at the second last position of SEQ ID NO: 2 is optionally substituted by L, and/or A at the last position of SEQ ID NO: 2 is optionally substituted by N.

In one embodiment, said recombinant binding protein comprises an ankyrin repeat domain having binding specificity for CD47, wherein said binding protein binds human CD47 in PBS with a dissociation constant (K_{D}) of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 3, and wherein A at the second last position of SEQ ID NO: 3 is optionally substituted by L, and/or A at the last position of SEQ ID NO: 3 is optionally substituted by N. Thus, in one embodiment, said recombinant binding protein binds human CD47 in PBS with a dissociation constant (K_{D}) of or below about 10⁻⁸M, and said ankyrin repeat domain comprises an amino acid sequence with at least 80% amino acid sequence identity with SEQ ID NO: 3. In one embodiment, said binding protein binds human CD47 in PBS with a dissociation constant (K_{D}) below 10⁻⁸M, and said ankyrin repeat domain comprises an amino acid sequence with at least 90% amino acid sequence identity with SEQ ID NO: 3. In another embodiment, said recombinant binding protein binds human CD47 in PBS with a dissociation constant (K_{D}) below 10⁻⁸M, and said ankyrin repeat domain comprises an amino acid sequence with at least 93% amino acid sequence identity with SEQ ID NO: 3; and in a further embodiment, said recombinant binding protein binds human CD47 in PBS with a dissociation constant (K_{D}) below 10⁻⁸M, and said ankyrin repeat domain comprises an amino acid sequence with at least 95% amino acid sequence identity with SEQ ID NO: 3. In one embodiment, said recombinant binding protein binds human CD47 in PBS with a dissociation constant (K_{D}) below 10⁻⁸M, and said ankyrin repeat domain comprises an amino acid sequence with at least 98% amino acid sequence identity with SEQ ID NO: 3; and in one embodiment, said recombinant binding protein binds human CD47 in PBS with a dissociation constant (K_{D}) below 10⁻⁸M, and said ankyrin repeat domain comprises the amino acid sequence of SEQ ID NO: 3. Thus, in one embodiment, said recombinant binding protein comprises an ankyrin repeat domain with binding specificity for CD47, wherein said recombinant binding protein binds human CD47 in PBS with a dissociation constant (K_{D}) of or below about 10⁻⁸M, and wherein said ankyrin repeat domain comprises the amino acid sequence of SEQ ID NO: 3, and wherein A at the second last position of SEQ ID NO: 3 is optionally substituted by L, and/or A at the last position of SEQ ID NO: 3 is optionally substituted by N.

In one embodiment, said recombinant binding protein comprises an ankyrin repeat domain having binding specificity for CD47, wherein said recombinant binding protein binds human CD47 in PBS with a dissociation constant (K_{D}) of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 4, and wherein A at the second last position of SEQ ID NO: 4 is optionally substituted by L, and/or A at the last position of SEQ ID NO: 4 is optionally substituted by N. Thus, in one embodiment, said recombinant binding protein binds human CD47 in PBS with a dissociation constant (K_{D}) of or below about 10⁻⁸M, and said ankyrin repeat domain comprises an amino acid sequence with at least 80% amino acid sequence identity with SEQ ID NO: 4. In one embodiment, said recombinant binding protein binds human CD47 in PBS with a dissociation constant (K_{D}) below 10⁻⁸M, and said ankyrin repeat domain comprises an amino acid sequence with at least 90% amino acid sequence identity with SEQ ID NO: 4. In another embodiment, said recombinant binding protein binds human CD47 in PBS with a dissociation constant (K_{D}) below 10⁻⁸M, and said ankyrin repeat domain comprises an amino acid sequence with at least 93% amino acid sequence identity with SEQ ID NO: 4; and in a further embodiment, said recombinant binding protein binds human CD47 in PBS with a dissociation constant (K_{D}) below 10⁻⁸M, and said ankyrin repeat domain comprises an amino acid sequence with at least 95% amino acid sequence identity with SEQ ID NO: 4. In one embodiment, said recombinant binding protein binds human CD47 in PBS with a dissociation constant (K_{D}) below 10⁻⁸M, and said ankyrin repeat domain comprises an amino acid sequence with at least 98% amino acid sequence identity with SEQ ID NO: 4; and in one embodiment, said recombinant binding protein binds human CD47 in PBS with a dissociation constant (K_{D}) below 10⁻⁸M, and said ankyrin repeat domain comprises the amino acid sequence of SEQ ID NO: 4. Thus, in one embodiment, said recombinant binding protein comprises an ankyrin repeat domain with binding specificity for CD47, wherein said recombinant binding protein binds human CD47 in PBS with a dissociation constant (K_{D}) of or below about 10⁻⁸M, and wherein said ankyrin repeat domain comprises the amino acid sequence of SEQ ID NO: 4, and wherein A at the second last position of SEQ ID NO: 4 is optionally substituted by L, and/or A at the last position of SEQ ID NO: 4 is optionally substituted by N.

In one embodiment, said recombinant binding protein comprises an ankyrin repeat domain having binding specificity for CD47, wherein said recombinant binding protein binds human CD47 in PBS with a dissociation constant (K_{D}) of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 5, and wherein A at the second last position of SEQ ID NO: 5 is optionally substituted by L, and/or A at the last position of SEQ ID NO: 5 is optionally substituted by N. Thus, in one embodiment, said recombinant binding protein binds human CD47 in PBS with a dissociation constant (K_{D}) of or below about 10⁻⁸M, and said ankyrin repeat domain comprises an amino acid sequence with at least 80% amino acid sequence identity with SEQ ID NO: 5. In one embodiment, said recombinant binding protein binds human CD47 in PBS with a dissociation constant (K_{D}) below 10⁻⁸M, and said ankyrin repeat domain comprises an amino acid sequence with at least 90% amino acid sequence identity with SEQ ID NO: 5. In another embodiment, said recombinant binding protein binds human CD47 in PBS with a dissociation constant (K_{D}) below 10⁻⁸M, and said ankyrin repeat domain comprises an amino acid sequence with at least 93% amino acid sequence identity with SEQ ID NO: 5; and in a further embodiment, said recombinant binding protein binds human CD47 in PBS with a dissociation constant (K_{D}) below 10⁻⁸M, and said ankyrin repeat domain comprises an amino acid sequence with at least 95% amino acid sequence identity with SEQ ID NO: 5. In one embodiment, said recombinant binding protein binds human CD47 in PBS with a dissociation constant (K_{D}) below 10⁻⁸M, and said ankyrin repeat domain comprises an amino acid sequence with at least 98% amino acid sequence identity with SEQ ID NO: 5; and in one embodiment, said recombinant binding protein binds human CD47 in PBS with a dissociation constant (K_{D}) below 10⁻⁸M, and said ankyrin repeat domain comprises the amino acid sequence of SEQ ID NO: 5. Thus, in one embodiment, said recombinant binding protein comprises an ankyrin repeat domain with binding specificity for CD47, wherein said recombinant binding protein binds human CD47 in PBS with a dissociation constant (K_{D}) of or below about 10⁻⁸M, and wherein said ankyrin repeat domain comprises the amino acid sequence of SEQ ID NO: 5, and wherein A at the second last position of SEQ ID NO: 5 is optionally substituted by L, and/or A at the last position of SEQ ID NO: 5 is optionally substituted by N.

In one embodiment, said recombinant binding protein comprises an ankyrin repeat domain having binding specificity for CD47, wherein said recombinant binding protein binds human CD47 in PBS with a dissociation constant (K_{D}) of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 6, and wherein A at the second last position of SEQ ID NO: 6 is optionally substituted by L, and/or A at the last position of SEQ ID NO: 6 is optionally substituted by N. Thus, in one embodiment, said recombinant binding protein binds human CD47 in PBS with a dissociation constant (K_{D}) of or below about 10⁻⁸M, and said ankyrin repeat domain comprises an amino acid sequence with at least 80% amino acid sequence identity with SEQ ID NO: 6. In one embodiment, said recombinant binding protein binds human CD47 in PBS with a dissociation constant (K_{D}) below 10⁻⁸M, and said ankyrin repeat domain comprises an amino acid sequence with at least 90% amino acid sequence identity with SEQ ID NO: 6. In another embodiment, said recombinant binding protein binds human CD47 in PBS with a dissociation constant (K_{D}) below 10⁻⁸M, and said ankyrin repeat domain comprises an amino acid sequence with at least 93% amino acid sequence identity with SEQ ID NO: 6; and in a further embodiment, said recombinant binding protein binds human CD47 in PBS with a dissociation constant (K_{D}) below 10⁻⁸M, and said ankyrin repeat domain comprises an amino acid sequence with at least 95% amino acid sequence identity with SEQ ID NO: 6. In one embodiment, said recombinant binding protein binds human CD47 in PBS with a dissociation constant (K_{D}) below 10⁻⁸M, and said ankyrin repeat domain comprises an amino acid sequence with at least 98% amino acid sequence identity with SEQ ID NO: 6; and in one embodiment, said recombinant binding protein binds human CD47 in PBS with a dissociation constant (K_{D}) below 10⁻⁸M, and said ankyrin repeat domain comprises the amino acid sequence of SEQ ID NO: 6. Thus, in one embodiment, said recombinant binding protein comprises an ankyrin repeat domain with binding specificity for CD47, wherein said recombinant binding protein binds human CD47 in PBS with a dissociation constant (K_{D}) of or below about 10⁻⁸M, and wherein said ankyrin repeat domain comprises the amino acid sequence of SEQ ID NO: 6, and wherein A at the second last position of SEQ ID NO: 6 is optionally substituted by L, and/or A at the last position of SEQ ID NO: 6 is optionally substituted by N.

In one embodiment, said recombinant binding protein comprises an ankyrin repeat domain having binding specificity for CD47, wherein said recombinant binding protein binds human CD47 in PBS with a dissociation constant (K_{D}) of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 7, and wherein A at the second last position of SEQ ID NO: 7 is optionally substituted by L, and/or A at the last position of SEQ ID NO: 7 is optionally substituted by N. Thus, in one embodiment, said recombinant binding protein binds human CD47 in PBS with a dissociation constant (K_{D}) of or below about 10⁻⁸M, and said ankyrin repeat domain comprises an amino acid sequence with at least 80% amino acid sequence identity with SEQ ID NO: 7. In one embodiment, said recombinant binding protein binds human CD47 in PBS with a dissociation constant (K_{D}) below 10⁻⁸M, and said ankyrin repeat domain comprises an amino acid sequence with at least 90% amino acid sequence identity with SEQ ID NO: 7. In another embodiment, said recombinant binding protein binds human CD47 in PBS with a dissociation constant (K_{D}) below 10⁻⁸M, and said ankyrin repeat domain comprises an amino acid sequence with at least 93% amino acid sequence identity with SEQ ID NO: 7; and in a further embodiment, said recombinant binding protein binds human CD47 in PBS with a dissociation constant (K_{D}) below 10⁻⁸M, and said ankyrin repeat domain comprises an amino acid sequence with at least 95% amino acid sequence identity with SEQ ID NO: 7. In one embodiment, said recombinant binding protein binds human CD47 in PBS with a dissociation constant (K_{D}) below 10⁻⁸M, and said ankyrin repeat domain comprises an amino acid sequence with at least 98% amino acid sequence identity with SEQ ID NO: 7; and in one embodiment, said recombinant binding protein binds human CD47 in PBS with a dissociation constant (K_{D}) below 10⁻⁸M, and said ankyrin repeat domain comprises the amino acid sequence of SEQ ID NO: 7. Thus, in one embodiment, said recombinant binding protein comprises an ankyrin repeat domain with binding specificity for CD47, wherein said recombinant binding protein binds human CD47 in PBS with a dissociation constant (K_{D}) of or below about 10⁻⁸M, and wherein said ankyrin repeat domain comprises the amino acid sequence of SEQ ID NO: 7, and wherein A at the second last position of SEQ ID NO: 7 is optionally substituted by L, and/or A at the last position of SEQ ID NO: 7 is optionally substituted by N.

In one embodiment, said recombinant binding protein comprises an ankyrin repeat domain having binding specificity for CD47, wherein said recombinant binding protein binds human CD47 in PBS with a dissociation constant (K_{D}) of or below about 10⁻⁸M, and wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 100% amino acid sequence identity with SEQ ID NO: 8, and wherein A at the second last position of SEQ ID NO: 8 is optionally substituted by L, and/or A at the last position of SEQ ID NO: 8 is optionally substituted by N. Thus, in one embodiment, said recombinant binding protein binds human CD47 in PBS with a dissociation constant (K_{D}) of or below about 10⁻⁸M, and said ankyrin repeat domain comprises an amino acid sequence with at least 80% amino acid sequence identity with SEQ ID NO: 8. In one embodiment, said recombinant binding protein binds human CD47 in PBS with a dissociation constant (K_{D}) below 10⁻⁸M, and said ankyrin repeat domain comprises an amino acid sequence with at least 90% amino acid sequence identity with SEQ ID NO: 8. In another embodiment, said recombinant binding protein binds human CD47 in PBS with a dissociation constant (K_{D}) below 10⁻⁸M, and said ankyrin repeat domain comprises an amino acid sequence with at least 93% amino acid sequence identity with SEQ ID NO: 8; and in a further embodiment, said recombinant binding protein binds human CD47 in PBS with a dissociation constant (K_{D}) below 10⁻⁸M, and said ankyrin repeat domain comprises an amino acid sequence with at least 95% amino acid sequence identity with SEQ ID NO: 8. In one embodiment, said recombinant binding protein binds human CD47 in PBS with a dissociation constant (K_{D}) below 10⁻⁸M, and said ankyrin repeat domain comprises an amino acid sequence with at least 98% amino acid sequence identity with SEQ ID NO: 8; and in one embodiment, said recombinant binding protein binds human CD47 in PBS with a dissociation constant (K_{D}) below 10⁻⁸M, and said ankyrin repeat domain comprises the amino acid sequence of SEQ ID NO: 8. Thus, in one embodiment, said recombinant binding protein comprises an ankyrin repeat domain with binding specificity for CD47, wherein said recombinant binding protein binds human CD47 in PBS with a dissociation constant (K_{D}) of or below about 10⁻⁸M, and wherein said ankyrin repeat domain comprises the amino acid sequence of SEQ ID NO: 8, and wherein A at the second last position of SEQ ID NO: 8 is optionally substituted by L, and/or A at the last position of SEQ ID NO: 8 is optionally substituted by N.

A typical and preferred determination of dissociation constants (K_{D}) of the inventive recombinant binding proteins with binding specificity for CD47 by Surface Plasmon Resonance (SPR) analysis is described in Example 3. Thus, in one embodiment said binding specificity for CD47 of the recombinant binding proteins comprising an ankyrin repeat domain is determined in PBS containing 0.005% Tween 20 by Surface Plasmon Resonance (SPR). In one embodiment said binding specificity for CD47 of the recombinant binding proteins comprising an ankyrin repeat domain is determined in PBS by Surface Plasmon Resonance (SPR) as described in Example 3.

In one embodiment, the invention relates to a recombinant binding protein comprising an ankyrin repeat domain with binding specificity for CD47, wherein said ankyrin repeat domain has a melting temperature (Tₘ) higher than 65° C., higher than 68° C., higher than 70° C., higher than 72° C., higher than 75° C., higher than 78° C., higher than 80° C., of about 75° C., of about 80° C., of about 82° C., of about 85° C., of between 65° C. and 95° C., of between 70° C. and 90° C., or of between 72° C. and 88° C. Thus, in one embodiment, said ankyrin repeat domain has a melting temperature (Tₘ) higher than 65° C. In another embodiment, said ankyrin repeat domain has a melting temperature (Tₘ) higher than 70° C. In another embodiment, said ankyrin repeat domain has a melting temperature (Tₘ) higher than 75° C. In another embodiment, said ankyrin repeat domain has a melting temperature (Tₘ) higher than 80° C. In another embodiment, said ankyrin repeat domain has a melting temperature (Tₘ) of between 65° C. and 95° C. In another embodiment, said ankyrin repeat domain has a melting temperature (Tₘ) of between 72° C. and 88° C. In one embodiment, said recombinant binding protein comprises an ankyrin repeat domain with binding specificity for CD47, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 1 to 8, wherein A at the second last position of SEQ ID NOs: 1 to 8 is optionally substituted by L, and/or A at the last position of SEQ ID NOs: 1 to 8 is optionally substituted by N; and wherein said ankyrin repeat domain has a melting temperature (Tₘ) higher than 65° C. In one embodiment, said ankyrin repeat domain has a melting temperature (Tₘ) higher than 68° C. In another embodiment, said ankyrin repeat domain has a melting temperature (Tₘ) higher than 70° C. In another embodiment, said ankyrin repeat domain has a melting temperature (Tₘ) higher than 72° C. In another embodiment, said ankyrin repeat domain has a melting temperature (Tₘ) higher than 75° C. In another embodiment, said ankyrin repeat domain has a melting temperature (Tₘ) higher than 78° C. In a further embodiment, said ankyrin repeat domain has a melting temperature (Tₘ) higher than 80° C.

In one embodiment, said recombinant binding protein comprises an ankyrin repeat domain with binding specificity for CD47, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 1, wherein A at the second last position of SEQ ID NO: 1 is optionally substituted by L, and/or A at the last position of SEQ ID NO: 1 is optionally substituted by N; and wherein said ankyrin repeat domain has a melting temperature (Tₘ) higher than 75° C. In one embodiment, said ankyrin repeat domain has a melting temperature (Tₘ) higher than 78° C. In one embodiment, said ankyrin repeat domain has a melting temperature (Tₘ) higher than 80° C. In one embodiment, said ankyrin repeat domain has a melting temperature (Tₘ) of between 75° C. and 90° C. In one embodiment, said ankyrin repeat domain has a melting temperature (Tₘ) of about 80° C.

In one embodiment, said recombinant binding protein comprises an ankyrin repeat domain with binding specificity for CD47, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 2, wherein A at the second last position of SEQ ID NO: 2 is optionally substituted by L, and/or A at the last position of SEQ ID NO: 2 is optionally substituted by N; and wherein said ankyrin repeat domain has a melting temperature (Tₘ) higher than 75° C. In one embodiment, said ankyrin repeat domain has a melting temperature (Tₘ) higher than 78° C. In one embodiment, said ankyrin repeat domain has a melting temperature (Tₘ) higher than 80° C. In one embodiment, said ankyrin repeat domain has a melting temperature (Tₘ) of between 75° C. and 90° C. In one embodiment, said ankyrin repeat domain has a melting temperature (Tₘ) of about 80° C.

In one embodiment, said recombinant binding protein comprises an ankyrin repeat domain with binding specificity for CD47, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 3, wherein A at the second last position of SEQ ID NO: 3 is optionally substituted by L, and/or A at the last position of SEQ ID NO: 3 is optionally substituted by N; and wherein said ankyrin repeat domain has a melting temperature (Tₘ) higher than 75° C. In one embodiment, said ankyrin repeat domain has a melting temperature (Tₘ) higher than 78° C. In one embodiment, said ankyrin repeat domain has a melting temperature (Tₘ) higher than 80° C. In one embodiment, said ankyrin repeat domain has a melting temperature (Tₘ) of between 75° C. and 90° C. In one embodiment, said ankyrin repeat domain has a melting temperature (Tₘ) of about 85° C. In one embodiment, said recombinant binding protein comprises an ankyrin repeat domain with binding specificity for CD47, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 4, wherein A at the second last position of SEQ ID NO: 4 is optionally substituted by L, and/or A at the last position of SEQ ID NO: 4 is optionally substituted by N; and wherein said ankyrin repeat domain has a melting temperature (Tₘ) higher than 75° C. In one embodiment, said ankyrin repeat domain has a melting temperature (Tₘ) higher than 78° C. In one embodiment, said ankyrin repeat domain has a melting temperature (Tₘ) higher than 80° C. In one embodiment, said ankyrin repeat domain has a melting temperature (Tₘ) of between 75° C. and 90° C. In one embodiment, said ankyrin repeat domain has a melting temperature (Tₘ) of about 80° C.

In one embodiment, said recombinant binding protein comprises an ankyrin repeat domain with binding specificity for CD47, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 5, wherein A at the second last position of SEQ ID NO: 5 is optionally substituted by L, and/or A at the last position of SEQ ID NO: 5 is optionally substituted by N; and wherein said ankyrin repeat domain has a melting temperature (Tₘ) higher than 75° C. In one embodiment, said ankyrin repeat domain has a melting temperature (Tₘ) higher than 78° C. In one embodiment, said ankyrin repeat domain has a melting temperature (Tₘ) higher than 80° C. In one embodiment, said ankyrin repeat domain has a melting temperature (Tₘ) of between 75° C. and 95° C. In one embodiment, said ankyrin repeat domain has a melting temperature (Tₘ) of about 90° C.

In one embodiment, said recombinant binding protein comprises an ankyrin repeat domain with binding specificity for CD47, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 6, wherein A at the second last position of SEQ ID NO: 6 is optionally substituted by L, and/or A at the last position of SEQ ID NO: 6 is optionally substituted by N; and wherein said ankyrin repeat domain has a melting temperature (Tₘ) higher than 75° C. In one embodiment, said ankyrin repeat domain has a melting temperature (Tₘ) higher than 78° C. In one embodiment, said ankyrin repeat domain has a melting temperature (Tₘ) higher than 80° C. In one embodiment, said ankyrin repeat domain has a melting temperature (Tₘ) of between 75° C. and 90° C. In one embodiment, said ankyrin repeat domain has a melting temperature (Tₘ) of about 85° C.

In one embodiment, said recombinant binding protein comprises an ankyrin repeat domain with binding specificity for CD47, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 7, wherein A at the second last position of SEQ ID NO: 7 is optionally substituted by L, and/or A at the last position of SEQ ID NO: 7 is optionally substituted by N; and wherein said ankyrin repeat domain has a melting temperature (Tₘ) higher than 75° C. In one embodiment, said ankyrin repeat domain has a melting temperature (Tₘ) higher than 78° C. In one embodiment, said ankyrin repeat domain has a melting temperature (Tₘ) higher than 80° C. In one embodiment, said ankyrin repeat domain has a melting temperature (Tₘ) of between 75° C. and 90° C. In one embodiment, said ankyrin repeat domain has a melting temperature (Tₘ) of about 85° C.

In one embodiment, said recombinant binding protein comprises an ankyrin repeat domain with binding specificity for CD47, wherein said ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 8, wherein A at the second last position of SEQ ID NO: 8 is optionally substituted by L, and/or A at the last position of SEQ ID NO: 8 is optionally substituted by N; and wherein said ankyrin repeat domain has a melting temperature (Tₘ) higher than 75° C. In one embodiment, said ankyrin repeat domain has a melting temperature (Tₘ) higher than 78° C. In one embodiment, said ankyrin repeat domain has a melting temperature (Tₘ) higher than 80° C. In one embodiment, said ankyrin repeat domain has a melting temperature (Tₘ) of between 75° C. and 95° C. In one embodiment, said ankyrin repeat domain has a melting temperature (Tₘ) of about 90° C.

A typical and preferred determination of the melting temperature (Tₘ) of the recombinant binding proteins comprising an ankyrin repeat domain with binding specificity for CD47 by Circular Dichroism (CD) spectroscopy is described in Example 2. Thus, in one embodiment, said melting temperature (Tₘ) of the recombinant binding proteins comprising an ankyrin repeat domain is determined in TBS pH 8.0 (50 mM Tris, 500 mM NaCl) by Circular Dichroism (CD) spectroscopy. In one embodiment, said melting temperature (Tₘ) of the recombinant binding proteins comprising an ankyrin repeat domains is determined in TBS pH 8.0 (50 mM Tris, 500 mM NaCl) by Circular Dichroism (CD) spectroscopy as described in Example 2.

The repeat domains, preferably ankyrin repeat domains, of the recombinant binding protein disclosed herein preferably comprise a N-terminal and/or a C-terminal capping module (thereafter also referred to as capping repeats or capping units). Capping modules are located at the N- and/or C-terminal end of an ankyrin repeat domain, typically forming tight tertiary interactions (i.e. tertiary structure interactions) with the ankyrin repeat module(s) in between, thereby providing a cap that shields the hydrophobic core of the ankyrin repeat domain at the side from exposure to the solvent. The N- and/or C-terminal capping modules may be derived from a capping unit or other structural unit found in a naturally occurring repeat protein adjacent to a repeat unit. Examples of capping sequences are described in International Patent Publication Nos. WO 2002/020565 and WO 2012/069655, in U.S. Patent Publication No. US20130296221, and by Interlandi et al., J Mol Biol. 2008 Jan. 18; 375 (3): 837-54. Examples of N-terminal capping modules (i.e. N-terminal capping repeats) are provided with SEQ ID NOs: 22 to 36 and examples of C-terminal capping modules (i.e. C-terminal capping repeats) are provided with SEQ ID NOs: 37 to 60.

In an exemplary embodiment, the N-terminal capping module comprises the amino acid sequence of any one of SEQ ID NOS: 22 to 36, wherein up to 9, up to 8, up to 7, up to 6, up to 5, up to 4, up to 3, up to 2 or up to 1 amino acid(s) of any one of SEQ ID NOs: 22 to 36 are optionally exchanged by any amino acids.

In an exemplary embodiment, the C-terminal capping module comprises the amino acid sequence of any one of SEQ ID NO: 37 to 60, wherein up to 9, up to 8, up to 7, up to 6, up to 5, up to 4, up to 3, up to 2 or up to 1 amino acid(s) of any one of SEQ ID NOS: 37 to 60 are optionally exchanged by any amino acids.

Advantageously, in some embodiments, certain amino acid residues in the N-terminal capping module and/or the C-terminal capping module of the designed ankyrin repeat domain herein provided are altered, resulting in improved pharmacokinetic properties, including a prolonged terminal half-life, of the designed ankyrin repeat domain and of the recombinant binding proteins comprising the designed ankyrin repeat domain. The altered amino acid residues are mostly surface exposed residues.

In a particular embodiment, the ankyrin repeat domain with binding specificity for CD47 comprises the amino acid sequence of any one of SEQ ID NOs: 1 to 8, wherein the amino acid E at position 8 of any one of SEQ ID NOs: 1 to 8 is substituted with Q and/or the amino acid D at position 15 of any one of SEQ ID NOs: 1 to 8 is substituted with L. Alternatively or additionally, the ankyrin repeat domain with binding specificity for CD47 comprises the amino acid sequence of any one of SEQ ID NOs: 1 to 8, wherein the amino acid K at position 110 of any one of SEQ ID NOS: 1 to 8 is substituted with R and/or the amino acid E at position 114 of SEQ ID NOs: 1 and 8 is substituted with Q.

In some aspects, amino acid substitution(s) made to the binding agents do not change the Ko value by more than about 1000-fold, more than about 100-fold, or more than about 10-fold, compared to the K_{D} value of the unsubstituted binding agents. For example, in some aspects, the amino acid substitution(s) do not change the K_{D} value by more than about 1000-fold, more than about 300-fold, more than about 100-fold, more than about 50-fold, more than about 25-fold, more than about 10-fold, or more than about 5-fold, compared to the K_{D} value of the binding agent comprising any of the sequences of SEQ ID NOs: 1 to 21.

In certain embodiments, the substitution is a conservative substitution according to Table 1. In certain embodiments, the substitution is made outside the structural core residues of the ankyrin repeat domain, e.g., in the beta loops that connect the alpha-helices.

TABLE 1
Amino acid substitutions

| Original | Conservative | Exemplary |
|---|---|---|
| Residue | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| lle (I) | Leu | Leu; Val; Met; Ala; Phe; Norleucine |
| Leu (L) | lle | Norleucine; lle; Val; Met; Ala; Phe |
| Lys (K) | Arg | Arg; Gln; Asn |
| Met (M) | Leu | Leu; Phe; lle |
| Phe (F) | Tyr | Leu; Val; lle; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr(Y) | Phe | Trp; Phe; Thr; Ser |
| Val (V) | Leu | lle; Leu; Met; Phe; Ala; Norleucine |

Furthermore, the CD47-binding domain of the invention may optionally further comprise a “G,” an “S,” or a “GS” sequence at its N-terminus. Accordingly, in some embodiments, the CD47-binding domain provided herein (i) comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOS: 1 to 8, and (ii) further comprises at its N-terminus a G, an S, or a GS. In an exemplary embodiment, the CD47-binding domain comprises an amino acid sequence that is at least 90% identical to any one of SEQ ID NOs: 1 to 8, and further comprises at its N-terminus a G, an S, or a GS. In an exemplary embodiment, the CD47-binding domain comprises an amino acid sequence that is at least 95% identical to any one of SEQ ID NOs: 1 to 8, and further comprises at its N-terminus a G, an S, or a GS. In an exemplary embodiment, the CD47-binding domain comprises the amino acid sequence of any one of SEQ ID NOS: 1 to 8, and further comprises at its N-terminus a G, an S, or a GS.

In another aspect of the invention, the recombinant binding protein with binding specificity for CD47 further comprises a binding agent with binding specificity for a protein expressed on the surface of an immune cell, preferably an NK cell or a macrophage. As an example, in one particular embodiment, a recombinant binding protein of the invention further comprises a binding agent with binding specificity for CD16a. In one embodiment, said binding agent with binding specificity for CD16a is a designed ankyrin repeat domain. Suitable designed ankyrin repeat domains with binding specificity for CD16a are described in EP23177833. In one embodiment said designed ankyrin repeat domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 81.

In the context of the present invention, a binding agent may be an antibody, an antibody mimetic, including a scaffold protein or a repeat protein, a designed repeat domain, preferably a designed ankyrin repeat domain, or any other suitable binding molecules known in the art. In one embodiment, said binding agent is an antibody. In another embodiment, said binding agent is a designed repeat domain, preferably a designed ankyrin repeat domain.

In one embodiment, said binding agent is linked, conjugated, fused or otherwise physically attached to said CD47-specific ankyrin repeat domain. In one embodiment, said binding agent is covalently linked to said CD47-specific ankyrin repeat domain. In one embodiment, said binding agent is covalently linked to said CD47-specific ankyrin repeat domain with a peptide linker. In one embodiment, said peptide linker is a proline-threonine rich peptide linker. In one embodiment, said peptide linker is the proline-threonine rich peptide linker of SEQ ID NO: 65. In one embodiment, said binding agent is covalently linked to said CD47-specific ankyrin repeat domain with the proline-threonine rich peptide linker of SEQ ID NO: 65. In another embodiment, said peptide linker is a glycine-serine rich peptide linker. In one embodiment, said peptide linker is the glycine-serine rich peptide linker of SEQ ID NO: 66. In one embodiment, said binding agent is covalently linked to said CD47-specific ankyrin repeat domain with the glycine-serine rich peptide linker of SEQ ID NO: 66.

In another aspect of the invention, the recombinant binding protein of the invention further comprises a binding agent with binding specificity for a tumor-associated antigen.

Thus, in a particular embodiment, the recombinant binding protein of the invention comprises an ankyrin repeat domain with binding specificity for CD47 as described more specifically in any of the aspects or embodiments herein and further comprises an additional binding agent with binding specificity for a tumor-associated antigen.

In one embodiment, said binding agent with binding specificity for a tumor-associated antigen is linked, conjugated, fused or otherwise physically attached to said ankyrin repeat domain with binding specificity for CD47. In one embodiment, said binding agent with binding specificity for a tumor-associated antigen is covalently linked to said ankyrin repeat domain with binding specificity for CD47. In one embodiment, said binding agent with binding specificity for a tumor-associated antigen is covalently linked to said ankyrin repeat domain with binding specificity for CD47 with a peptide linker. In one embodiment, the amino acid sequence of said peptide linker has a length from 1 to 50 amino acids, preferably from 6 to 38 amino acids. In one embodiment, said peptide linker is a proline-threonine rich peptide linker or glycine-serine rich linker. In one embodiment, said peptide linker is the proline-threonine rich peptide linker of SEQ ID NO:65 or the glycine-serine rich linker of SEQ ID NO: 66. In one embodiment, said binding agent with binding specificity for a tumor-associated antigen is covalently linked to said ankyrin repeat domain with binding specificity for CD47 with the proline-threonine rich peptide linker of SEQ ID NO: 65 or the glycine-serine rich linker of SEQ ID NO: 66. In one preferred embodiment, said binding agent with binding specificity for a tumor-associated antigen is a designed ankyrin repeat domain with binding specificity for a disease-associated antigen.

In one embodiment, the recombinant binding protein of the invention comprises an ankyrin repeat domain with binding specificity for CD47 as described more specifically in any of the aspects or embodiments herein and further comprises two or more ankyrin repeat domains with binding specificity for a tumor-associated antigen as described more specifically in any of the aspects or embodiments herein.

In one embodiment, the recombinant binding protein of the invention comprises an ankyrin repeat domain with binding specificity for CD47, an ankyrin repeat domain with binding specificity for one or more tumor associated antigen(s) and/or a binding agent with binding specificity for a protein expressed on the surface of an immune cell, preferably an NK cell or a macrophage.

In one embodiment, said recombinant binding protein comprises an ankyrin repeat domain with binding specificity for CD47 as described herein and further comprises an ankyrin repeat domain with dual binding specificity, wherein binding of said ankyrin repeat domain with dual binding specificity to its first and second binding targets is mutually exclusive. Such ankyrin repeat domains with dual binding specificity (also referred to as “2-in-1 repeat domains”) and corresponding production methods are described in PCT/EP2022/085794. In a more particular embodiment, said recombinant binding protein comprises an ankyrin repeat domain with binding specificity for CD47 as described herein and further comprises a second ankyrin repeat domain, wherein the second ankyrin repeat domain is a 2-in-1 repeat domain having a first binding specificity for a TAA and a second binding specificity for said first ankyrin repeat domain having binding specificity for CD47. Such a design enables a conditional TAA-dependent activation of the CD47 binding domain, wherein upon presence of the TAA, the 2-in-1 repeat domain binds the TAA and releases the CD47-specific binding domain from binding to the 2-in-1 repeat domain, thereby releasing the inhibition of the CD47-specific binding domain.

In one aspect of the invention, the recombinant binding protein further comprises a half-life extending moiety.

Thus, in a particular embodiment, the recombinant binding protein of the invention comprises an ankyrin repeat domain with binding specificity for CD47 as described more specifically in any of the aspects or embodiments herein and further comprises a half-life extending moiety. In a particular embodiment the recombinant binding protein of the invention comprises an ankyrin repeat domain with binding specificity for CD47, an ankyrin repeat domain with binding specificity for one or more tumor associated antigen(s) and/or a binding agent with binding specificity for a protein expressed on the surface of an immune cell, preferably an NK cell or a macrophage, and further comprises a half-life extending moiety. In one embodiment said binding agent with binding specificity for said TAA and/or said protein expressed on the surface of an immune cell, preferably an NK cell or a macrophage, more preferably said protein being CD16a, comprises or is an ankyrin repeat domain. In one embodiment said designed ankyrin repeat domain with binding specificity for CD16a comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 81.

In one embodiment, said half-life extending moiety comprises a binding agent with binding specificity for human serum albumin.

In one embodiment, said half-life extending moiety is a designed ankyrin repeat domain with binding specificity for human serum albumin. In one embodiment, said designed ankyrin repeat domain with binding specificity for human serum albumin comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with any one of SEQ ID NOs: 62 to 64. Thus, in one embodiment, the recombinant binding protein of the invention comprises an ankyrin repeat domain with binding specificity for CD47 as described more specifically in any of the aspects or embodiments herein and further comprises an ankyrin repeat domain with binding specificity for human serum albumin having an amino acid sequence with at least 80% amino acid sequence identity with any one of SEQ ID NOs: 62 to 64. In one embodiment, said ankyrin repeat domain with binding specificity for human serum albumin comprises an amino acid sequence with at least 90% amino acid sequence identity with any one of SEQ ID NOs: 62 to 64. In another embodiment, said ankyrin repeat domain with binding specificity for human serum albumin comprises an amino acid sequence with at least 93% amino acid sequence identity with any one of SEQ ID NOs: 62 to 64; and in a further embodiment, said ankyrin repeat domain with binding specificity for human serum albumin comprises an amino acid sequence with at least 95% amino acid sequence identity with any one of SEQ ID NOs: 62 to 64. In one embodiment, said ankyrin repeat domain with binding specificity for human serum albumin comprises an amino acid sequence with at least 98% amino acid sequence identity with any one of SEQ ID NOS: 62 to 64; and in a further embodiment, said ankyrin repeat domain with binding specificity for human serum albumin comprises the amino acid sequence of any one of SEQ ID NOs: 62 to 64.

Thus, in one embodiment, the recombinant binding protein of the invention comprises an ankyrin repeat domain with binding specificity for CD47 and further comprises an ankyrin repeat domain with binding specificity for human serum albumin, and wherein said ankyrin repeat domain with binding specificity for CD47 comprises the amino acid sequence of any one of SEQ ID NOS: 1 to 8 and wherein said ankyrin repeat domain with binding specificity for human serum albumin comprises the amino acid sequence of any one of SEQ ID NOs: 62 to 64. In a preferred embodiment, said ankyrin repeat domain with binding specificity for human serum albumin is located N-terminally of said ankyrin repeat domain with binding specificity for CD47 within said binding protein.

In a preferred embodiment, said designed ankyrin repeat domain with binding specificity for human serum albumin comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% amino acid sequence identity with SEQ ID NO: 63. Thus, in one embodiment, the recombinant binding protein of the invention comprises an ankyrin repeat domain with binding specificity for CD47 as described more specifically in any of the aspects or embodiments herein and further comprises an ankyrin repeat domain with binding specificity for human serum albumin comprising an amino acid sequence with at least 80% amino acid sequence identity with SEQ ID NO: 63. In one embodiment, said ankyrin repeat domain with binding specificity for human serum albumin comprises an amino acid sequence with at least 90% amino acid sequence identity with SEQ ID NO: 63. In another embodiment, said ankyrin repeat domain with binding specificity for human serum albumin comprises an amino acid sequence with at least 93% amino acid sequence identity with SEQ ID NO: 63; and in a further embodiment, said ankyrin repeat domain with binding specificity for human serum albumin comprises an amino acid sequence with at least 95% amino acid sequence identity with SEQ ID NO: 63. In one embodiment, said ankyrin repeat domain with binding specificity for human serum albumin comprises an amino acid sequence with at least 98% amino acid sequence identity with SEQ ID NO: 63; and in a further embodiment, said ankyrin repeat domain with binding specificity for human serum albumin comprises the amino acid sequence of SEQ ID NO: 63. Thus, in one embodiment, the recombinant binding protein of the invention comprises an ankyrin repeat domain with binding specificity for CD47 and further comprises an ankyrin repeat domain with binding specificity for human serum albumin, and wherein ankyrin repeat domain with binding specificity for CD47 comprises the amino acid sequence of any one of SEQ ID NOS: 1 to 8, and wherein said ankyrin repeat domain with binding specificity for human serum albumin comprises the amino acid sequences of SEQ ID NO: 63. In a preferred embodiment, said ankyrin repeat domain with binding specificity for human serum albumin is located N-terminally of said ankyrin repeat domain with binding specificity for CD47.

In one embodiment, a recombinant binding protein of the present invention comprising an ankyrin repeat domain with binding specificity for CD47 and further comprising an ankyrin repeat domain with binding specificity for human serum albumin exhibits an increased terminal half-life, preferably an increased terminal half-life of at least 5%, preferably 10%, 15%, 20%, 25%, 30%, 35%, 40%, 45%, 50%, 60%, 70%, 80%, 90%, 100%, 150%, 200%, or 250%, compared to a corresponding recombinant binding protein comprising said ankyrin repeat domain with binding specificity for CD47 but not comprising said ankyrin repeat domain with binding specificity for human serum albumin.

In one embodiment, the recombinant binding protein of the invention comprises an ankyrin repeat domain having binding specificity for CD47 as described more specifically in any of the aspects or embodiments herein and further comprises two ankyrin repeat domains with binding specificity for human serum albumin as described more specifically in any of the aspects or embodiments herein.

In some embodiments, the half-life extending moiety comprises an immunoglobulin domain. In some embodiments, the immunoglobulin domain comprises an Fc domain. In some embodiments, the Fc domain is derived from any one of the known heavy chain isotypes: IgG (γ), IgM (μ), IgD (δ), IgE (ε), or IgA (α). In some embodiments, the Fc domain is derived from any one of the known heavy chain isotypes or subtypes: IgG₁ (γ1), IgG₂ (γ2), IgG₃ (γ3), IgG₄ (γ4), IgA₁ (α1), IgA₂ (α2). In some embodiments, the Fc domain is the Fc domain of human IgG₁.

In some embodiments, the Fc domain comprises an uninterrupted native sequence (i.e., wild type sequence) of an Fc domain. In some embodiments, the immunoglobulin Fc domain comprises a variant Fc domain resulting in altered biological activity. For example, at least one point mutation or deletion may be introduced into the Fc domain so as to reduce or eliminate the effector activity (e.g., International Patent Publication No. WO 2005/063815), and/or to increase the homogeneity during the production of the recombinant binding protein. In some embodiments, the Fc domain is the Fc domain of human IgG1 and comprises one or more of the following effector-null substitutions: L234A, L235A, and G237A (Eu numbering). In some embodiments, the Fc domain does not comprise the lysine located at the C-terminal position of human IgG1 (i.e., K447 by Eu numbering). The absence of the lysine may increase homogeneity during the production of the recombinant binding protein. In some embodiments, the Fc domain comprises the lysine located at the C-terminal position (K447, Eu numbering).

In one embodiment, the recombinant binding protein of the invention further comprises a polypeptide tag. A polypeptide tag is an amino acid sequence attached to a polypeptide/protein, wherein said amino acid sequence is useful for the purification, detection, and/or targeting of said polypeptide/protein, and/or wherein said amino acid sequence improves the physicochemical behavior of the polypeptide/protein, and/or wherein said amino acid sequence possesses an effector function. The individual polypeptide tags of a binding protein may be connected to other parts of the binding protein directly or via a peptide linker. Polypeptide tags are all known in the art. Examples of polypeptide tags are small polypeptide sequences, for example, His, HA, myc, FLAG, or Strep-tags, or polypeptides such as enzymes (for example alkaline phosphatase), which allow the detection of said polypeptide/protein, or polypeptides which can be used for targeting (such as immunoglobulins or fragments thereof) and/or as effector molecules.

In one embodiment, the recombinant binding protein of the invention further comprises a peptide linker. A peptide linker is an amino acid sequence, which is able to link, for example, two protein domains, a polypeptide tag and a protein domain, a protein domain and a non-proteinaceous compound or polymer such as polyethylene glycol, a protein domain and a biologically active molecule, a protein domain and a localizer, or two sequence tags. Peptide linkers are known to the person skilled in the art. A list of examples is provided in the description of patent application WO2002/020565. Particular examples of such linkers are glycine-serine-linkers and proline-threonine-linkers of variable lengths. Examples of a glycine-serine-linker are the amino acid sequence GS and the amino acid sequence of SEQ ID NO: 66, and an example of a proline-threonine-linker is the amino acid sequences of SEQ ID NO: 65.

In another aspect, the invention relates to a nucleic acid encoding the amino acid sequence of an ankyrin repeat domain or a recombinant binding protein of the present invention. In one embodiment, the invention relates to a nucleic acid encoding the amino acid sequence of a recombinant binding protein of the present invention. In one embodiment, the invention relates to a nucleic acid encoding an amino acid sequence selected from the group consisting of SEQ ID NO: 1 to 8. In one embodiment, the invention relates to a nucleic acid encoding the amino acid sequence of SEQ ID NO: 1. In one embodiment, the invention relates to a nucleic acid encoding the amino acid sequence of SEQ ID NO: 2. In one embodiment, the invention relates to a nucleic acid encoding the amino acid sequence of SEQ ID NO: 3. In one embodiment, the invention relates to a nucleic acid encoding the amino acid sequence of SEQ ID NO: 4. In one embodiment, the invention relates to a nucleic acid encoding the amino acid sequence of SEQ ID NO: 5. In one embodiment, the invention relates to a nucleic acid encoding the amino acid sequence of SEQ ID NO: 6.

In one embodiment, the invention relates to a nucleic acid encoding the amino acid sequence of SEQ ID NO: 7. In one embodiment, the invention relates to a nucleic acid encoding the amino acid sequence of SEQ ID NO: 8. In another embodiment, the invention relates to a nucleic acid sequence encoding the amino acid sequence of a recombinant protein comprising an ankyrin repeat domain comprising any one of the amino acid sequences with SEQ ID NOs: 9-21.

Furthermore, the invention relates to vectors comprising any nucleic acid of the invention. Nucleic acids are well known to the skilled person in the art. In the examples, nucleic acids were used to produce designed ankyrin repeat domains or recombinant binding proteins of the invention in E. coli. Examples nucleic acids of the invention are provided by SEQ ID NOs: 73 to 80 which encode the amino acid sequences of SEQ ID NOs: 1 to 8, respectively.

In one aspect, the invention relates to a pharmaceutical composition comprising a recombinant binding protein and/or a designed ankyrin repeat domain of the present invention, and/or a nucleic acid encoding a recombinant binding protein and/or a designed ankyrin repeat domain of the present invention, and optionally a pharmaceutically acceptable carrier and/or diluent.

In one embodiment, the invention relates to a pharmaceutical composition comprising a recombinant binding protein or a nucleic acid encoding a recombinant binding protein of the present invention, and optionally a pharmaceutically acceptable carrier and/or diluent.

Pharmaceutically acceptable carriers and/or diluents are known to the person skilled in the art and are explained in more detail below.

A pharmaceutical composition comprises a recombinant binding protein, and/or a designed ankyrin repeat domain, and/or a nucleic acid, preferably a recombinant binding protein and/or a nucleic acid, as described herein and a pharmaceutically acceptable carrier, excipient or stabilizer, for example as described in Remington's Pharmaceutical Sciences 16^{th} edition, Osol, A. Ed., 1980.

Suitable carriers, excipients or stabilizers known to one of skill in the art include, for example, saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polylactic acids, polyglycolic acids, polymeric amino acids and amino acid copolymers. A pharmaceutical composition may also be a combination formulation, comprising an additional active agent, such as an anti-cancer agent or an anti-angiogenic agent, or an additional bioactive compound.

The formulations to be used for in vivo administration must be aseptic or sterile. This is readily accomplished by filtration through sterile filtration membranes.

One embodiment of the present invention relates to the use of a recombinant binding protein of the present invention comprising an ankyrin repeat domain with binding specificity for CD47 and further comprising an ankyrin repeat domain with binding specificity for human serum albumin for manufacturing a pharmaceutical composition, wherein said recombinant binding protein exhibits an increased terminal half-life, preferably an increased terminal half-life of at least 5%, preferably 10%, 15%, 20%, 25%, 30%, 35%, 40%, 45%, 50%, 60%, 70%, 80%, 90%, 100%, 150%, 200%, or 250%, compared to a corresponding recombinant binding protein comprising said ankyrin repeat domain with binding specificity for CD47 but not comprising said ankyrin repeat domain with binding specificity for serum albumin. In one embodiment of the invention, a recombinant binding protein comprises an ankyrin repeat domain having binding specificity for CD47 and further comprises two ankyrin repeat domains with binding specificity for serum albumin.

In one embodiment, a pharmaceutical composition comprises at least one recombinant binding protein as described herein and a detergent such as nonionic detergent, a buffer such as phosphate buffer, and a sugar such as sucrose. In one embodiment, such a composition comprises recombinant binding proteins as described above and PBS.

In one embodiment, the invention relates to a pharmaceutical composition, a recombinant binding protein, or a nucleic acid according to the present invention for use in the treatment of a disease. In one embodiment said disease is cancer. For that purpose, the pharmaceutical composition, the nucleic acid or the recombinant binding protein according to the present invention is administered to a subject in need thereof in a therapeutically effective amount. Administration may include topical administration, oral administration, and parenteral administration. The typical route of administration is parenteral administration. In parental administration, the pharmaceutical composition of this invention will be formulated in a unit dosage injectable form such as a solution, suspension or emulsion, in association with the pharmaceutically acceptable excipients as defined above. The dosage and mode of administration will depend on the individual to be treated and the particular disease.

Further, any of the above-mentioned pharmaceutical composition, nucleic acid or recombinant binding protein is considered for use in the treatment of a disorder. In one embodiment said disorder is cancer.

In one embodiment, said recombinant binding protein or such other pharmaceutical composition described herein is applied intravenously. For parenteral application, the recombinant binding protein or said pharmaceutical composition can be injected as bolus injection or by slow infusion at a therapeutically effective amount.

In one embodiment, the invention relates to the use of the recombinant binding protein of the invention, the nucleic acid of the invention or the pharmaceutical composition of the invention for manufacturing of a medicament. In one embodiment, the invention relates to the use of the recombinant binding protein of the invention, the nucleic acid of the invention or the pharmaceutical composition of the invention, for manufacturing of a medicament for the treatment of a disease. In one embodiment, the invention relates to a process for the manufacturing of a medicament for the treatment of a disease, wherein the recombinant binding protein of the invention, the nucleic acid of the invention or the pharmaceutical composition of the invention is an active ingredient of the medicament. In one embodiment, the invention relates to a method of treatment of a disease using the recombinant binding protein of the invention, the nucleic acid of the invention or the pharmaceutical composition of the invention.

The use of a recombinant binding protein of the present invention, a nucleic acid of the invention or a pharmaceutical composition of the invention for the treatment of cancer can also be in combination with one or more other therapies known in the art. The term “use in combination with”, as used herein, shall refer to a co-administration, which is carried out under a given regimen. This includes synchronous administration of the different compounds as well as time-shifted administration of the different compounds (e.g. compound A is given once and compound B is given several times thereafter, or vice versa, or both compounds are given synchronously and one of the two is also given at later stages).

In another aspect, the invention provides a method for treating a medical condition in a human subject, the method comprising administering to said subject a therapeutically effective amount of the recombinant binding protein of the invention, the nucleic acid of the invention or the pharmaceutical composition of the invention. In one embodiment said medical condition is cancer.

In one particular embodiment, the medical condition is cancer, wherein the cancer or tumor tissue comprises cells that express or display a tumor-associated antigen, and said binding agent with binding specificity for a tumor-associated antigen binds said tumor-associated antigen expressed or displayed in said cells. In one particular embodiment, said tumor-associated antigen is the extracellular domain of a cell surface protein expressed or overexpressed in and/or on and/or by said cancer or tumor tissue.

In another aspect, the invention provides a method of tumor-localized activation of immune cells, in a mammal, preferably a human, the method comprising the step of administering to said mammal the CD47-specific recombinant binding protein or nucleic acid of the invention, wherein said binding protein further comprises a binding agent with binding specificity for a protein expressed on the surface of said immune cell.

In another aspect, the invention provides a method of targeting tumor cells in a subject having a tumor, the method comprising the step of administering to the subject a therapeutically effective amount of the CD47-specific recombinant binding protein, nucleic acid, or pharmaceutical composition of the invention, wherein said first target peptide and/or, if said binding protein comprises a second repeat domain, said second target peptide is derived from a protein expressed in the tumor cells, preferably a protein expressed in the tumor cells. In one embodiment, said binding protein further comprises a toxic agent capable of killing a tumor cell.

In another aspect, the invention provides a method of diagnosing a medical condition in a mammal, preferably a human, the method comprising the steps of:
(i) contacting a cell or tissue sample obtained from said mammal with the CD47-specific recombinant binding protein of the invention; and
(ii) detecting specific binding of said recombinant binding protein to said cell or tissue sample.

In one embodiment, the invention relates to a kit comprising the recombinant binding protein of the invention. In one embodiment, the invention relates to a kit comprising a nucleic acid encoding the recombinant binding protein of the invention. In one embodiment, the invention relates to a kit comprising the pharmaceutical composition of the invention. In one embodiment, the invention relates to a kit comprising the recombinant binding protein of the invention, and/or the nucleic acid of the invention, and/or the pharmaceutical composition of the invention. In one embodiment, the invention relates to a kit comprising the recombinant binding protein comprising an ankyrin repeat domain with binding specificity for CD47 of the invention, for example SEQ ID NOs: 1 to 8 and/or a nucleic acid encoding the recombinant binding protein comprising an ankyrin repeat domain with binding specificity for CD47, for example SEQ ID NOs: 1 to 8, and/or a pharmaceutical composition comprising the recombinant binding protein comprising an ankyrin repeat domain with binding specificity for CD47, for example SEQ ID NOs: 1 to 8.

In one embodiment, the invention relates to a method for producing a recombinant binding protein of the present invention. In one embodiment, the invention relates to a method for producing a recombinant binding protein, for example a recombinant binding protein comprising the amino acid sequence of SEQ ID NOs: 1 to 8, the method comprising the steps of (i) expressing said recombinant binding protein in a suitable host cell (e.g., bacteria), and (ii) purifying said recombinant binding protein (e.g., using chromatography). Said method may comprise additional steps. Such a method of producing a recombinant binding protein of the present invention is described in Example 1.

The invention is not restricted to the embodiments described in the Examples.

This specification refers to amino acid sequences, nucleic acid sequences and SEQ ID NOs: 1 to 88 disclosed in the appended Sequence Listing, which is herewith incorporated by reference in its entirety.

### Definitions

Unless defined otherwise herein, all technical and scientific terms used herein shall have meanings that are commonly understood by those of ordinary skill in the art to which the present invention belongs.

As used in the specification and claims, the singular form “a”, “an” and “the” include plural references unless the context clearly dictates otherwise.

Throughout this specification and the claims which follow, unless the context requires otherwise, the word “comprise”, and variations such as “comprises” and “comprising”, are used herein in their open-ended and non-limiting sense unless otherwise noted.

In each instance herein any of the terms “comprising”, “consisting essentially of” and “consisting of” may be replaced with either of the other two terms.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely examples and that equivalents of such are known in the state of the art.

In the context of the present invention the term “protein” refers to a molecule comprising a polypeptide, wherein at least part of the polypeptide has, or is able to acquire, a defined three-dimensional arrangement by forming secondary, tertiary, and/or quaternary structures within a single polypeptide chain and/or between multiple polypeptide chains. If a protein comprises two or more polypeptide chains, the individual polypeptide chains may be linked non-covalently or covalently, e.g. by a disulfide bond between two polypeptides. A part of a protein, which individually has, or is able to acquire, a defined three-dimensional arrangement by forming secondary and/or tertiary structure, is termed “protein domain”. Such protein domains are known to the practitioner skilled in the art.

The term “recombinant” as used in recombinant protein, recombinant polypeptide and the like, means that said protein or polypeptide is produced by the use of recombinant DNA technologies known to the practitioner skilled in the art. For example, a recombinant DNA molecule (e.g. produced by gene synthesis) encoding a polypeptide can be cloned into a bacterial expression plasmid (e.g. pQE30, QIAgen), yeast expression plasmid, mammalian expression plasmid, or plant expression plasmid, or a DNA enabling in vitro expression. If, for example, such a recombinant bacterial expression plasmid is inserted into appropriate bacteria (e.g. Escherichia coli), these bacteria can produce the polypeptide(s) encoded by this recombinant DNA. The correspondingly produced polypeptide or protein is called a recombinant polypeptide or recombinant protein.

In the context of the present invention, the term “binding protein” refers to a protein comprising a binding domain. A binding protein may also comprise two, three, four, five or more binding domains. Preferably, said binding protein is a recombinant binding protein. Binding proteins of the instant invention comprise an ankyrin repeat domain with binding specificity for CD47.

The term “CD47”, also known as Integrin-Associated Protein (IAP), refers to leukocyte surface antigen CD47, a transmembrane protein encoded by the human gene CD47. The amino acid sequence of human CD47 is shown in UniProt (www.uniprot.org) Ref. No. Q08722.

The term “CD16a” refers to the Fc gamma III-A receptor. This is a receptor for IgGs. The amino acid sequence of human CD16a (hCD16a) is shown in UniProt (www.uniprot.org) Ref. No. P08637.

Furthermore, any such binding protein may comprise additional polypeptides (such as e.g. polypeptide tags, peptide linkers, fusion to other proteinaceous domains with binding specificity, cytokines, hormones, or antagonists), or chemical modifications (such as coupling to polyethylene-glycol, toxins (e.g. DM1 from Immunogen), small molecules, antibiotics and alike) known to the person skilled in the art.

The term “binding domain” means a protein domain exhibiting binding specificity for a target. Preferably, said binding domain is a recombinant binding domain.

The term “target” refers to an individual molecule such as a nucleic acid molecule, a polypeptide or protein, a carbohydrate, or any other naturally occurring molecule, including any part of such individual molecule, or to complexes of two or more of such molecules, or to a whole cell or a tissue sample, or to any non-natural compound. Preferably, a target is a naturally occurring or non-natural polypeptide or protein, or a polypeptide or protein containing chemical modifications, for example, naturally occurring or non-natural phosphorylation, acetylation, or methylation. In the context of the present invention, CD47 expressed in cancer cells is the target of CD47-specific binding proteins.

In the context of the present invention, the term “polypeptide” relates to a molecule consisting of a chain of multiple, i.e. two or more, amino acids linked via peptide bonds. Preferably, a polypeptide consists of more than eight amino acids linked via peptide bonds. The term “polypeptide” also includes multiple chains of amino acids, linked together by S—S bridges of cysteines. Polypeptides are known to the person skilled in the art.

Patent application WO2002/020565 and Forrer et al., 2003 (Forrer, P., Stumpp, M. T., Binz, H. K., Plückthun, A., 2003. FEBS Letters 539, 2-6), contain a general description of repeat protein features and repeat domain features, techniques and applications. The term “repeat protein” refers to a protein comprising one or more repeat domains. Preferably, a repeat protein comprises one, two, three, four, five or six repeat domains. Furthermore, said repeat protein may comprise additional non-repeat protein domains, polypeptide tags and/or peptide linkers. The repeat domains can be binding domains.

The term “repeat domain” refers to a protein domain comprising two or more consecutive repeat modules as structural units, wherein said repeat modules have structural and sequence homology. Preferably, a repeat domain further comprises an N-terminal and/or a C-terminal capping module. For clarity, a capping module can be a repeat module. Such repeat domains, repeat modules, and capping modules, sequence motives, as well as structural homology and sequence homology are well known to the practitioner in the art from examples of ankyrin repeat domains (WO2002/020565), leucine-rich repeat domains (WO2002/020565), tetratricopeptide repeat domains (Main, E. R., Xiong, Y., Cocco, M. J., D'Andrea, L., Regan, L., Structure 11 (5), 497-508, 2003), and armadillo repeat domains (WO2009/040338). It is further known to the practitioner in the art that such repeat domains are different from proteins comprising repeated amino acid sequences, where every repeated amino acid sequence is able to form an individual domain (for example FN3 domains of Fibronectin).

The term “designed” as used in designed repeat protein, designed repeat domain and the like refers to the property that such repeat proteins and repeat domains, respectively, are man-made and do not occur in nature. The recombinant binding proteins of the instant invention are designed repeat proteins and they comprise at least one designed ankyrin repeat domain.

The term “target interaction residues” refers to amino acid residues of a repeat module, which contribute to the direct interaction with a target.

The term “framework residues” (or “framework positions”) refers to amino acid residues of a repeat module, which contribute to the folding topology, i.e. which contribute to the fold of said repeat module or which contribute to the interaction with a neighboring module. Such contribution may be the interaction with other residues in the repeat module, or the influence on the polypeptide backbone conformation as found in α-helices or β-sheets, or the participation in amino acid stretches forming linear polypeptides or loops.

Such framework and target interaction residues may be identified by analysis of the structural data obtained by physicochemical methods, such as X-ray crystallography, NMR and/or CD spectroscopy, or by comparison with known and related structural information known to practitioners in structural biology and/or bioinformatics.

The term “repeat modules” refers to the repeated amino acid sequence and structural units of the designed repeat domains, which are originally derived from the repeat units of naturally occurring repeat proteins. Each repeat module comprised in a repeat domain is derived from one or more repeat units of a family or subfamily of naturally occurring repeat proteins, e.g. the family of ankyrin repeat proteins. Furthermore, each repeat module comprised in a repeat domain may comprise a “repeat sequence motif” deduced from homologous repeat modules obtained from repeat domains selected on a target, e.g. as described in Example 1, and having the same target specificity.

Accordingly, the term “ankyrin repeat module” refers to a repeat module, which is originally derived from the repeat units of naturally occurring ankyrin repeat proteins. Ankyrin repeat proteins are known to the person skilled in the art.

Repeat modules may comprise positions with amino acid residues which have not been randomized in a library for the purpose of selecting target-specific repeat domains (“non-randomized positions”) and positions with amino acid residues which have been randomized in the library for the purpose of selecting target-specific repeat domains (“randomized positions”). The non-randomized positions comprise framework residues. The randomized positions comprise target interaction residues. “Have been randomized” means that two or more amino acids were allowed at an amino acid position of a repeat module, for example, wherein any of the usual twenty naturally occurring amino acids were allowed, or wherein most of the twenty naturally occurring amino acids were allowed, such as amino acids other than cysteine, or amino acids other than glycine, cysteine and proline. For the purpose of this patent application, amino acid residues 3, 4, 6, 11, 14 and 15 of SEQ ID NOs: 9 to 21, amino acid residues 4, 8, 11 and 12 of SEQ ID NOs: 22 to 36, and amino acid residues 3, 4, 6, 14 and 15 of SEQ ID NOs: 37 to 60 are randomized positions of the ankyrin repeat modules of the instant invention. Randomized positions are generally not substituted in the variants of the module sequences disclosed herein.

The term “repeat sequence motif” refers to an amino acid sequence, which is deduced from one or more repeat modules. Preferably, said repeat modules are from repeat domains having binding specificity for the same target. Such repeat sequence motifs comprise framework residue positions and target interaction residue positions. Said framework residue positions correspond to the positions of framework residues of the repeat modules. Likewise, said target interaction residue positions correspond to the positions of target interaction residues of the repeat modules. Repeat sequence motifs comprise non-randomized positions and randomized positions.

The term “repeat unit” refers to amino acid sequences comprising sequence motifs of one or more naturally occurring proteins, wherein said “repeat units” are found in multiple copies, and exhibit a defined folding topology common to all said motifs determining the fold of the protein. Examples of such repeat units include leucine-rich repeat units, ankyrin repeat units, armadillo repeat units, tetratricopeptide repeat units, HEAT repeat units, and leucine-rich variant repeat units.

The term “binding specificity”, “has binding specificity for a target”, “specifically binding to a target”, “binding to a target with high specificity”, “specific for a target” or “target specificity” and the like means that a binding protein or binding domain binds to a target with a lower dissociation constant (i.e. it binds with higher affinity) than it binds to an unrelated protein such as the E. coli maltose binding protein (MBP). Preferably, the dissociation constant (“K_{D}”) for the target is at least 102; more preferably, at least 103; more preferably, at least 104; or more preferably, at least 105 times lower than the corresponding dissociation constant for MBP. Methods to determine dissociation constants of protein-protein interactions, such as surface plasmon resonance (SPR) based technologies (e.g. SPR equilibrium analysis) or isothermal titration calorimetry (ITC) are known to the person skilled in the art. The measured K_{D} values of a particular protein-protein interaction can vary if measured under different conditions (e.g., salt concentration, pH). Thus, measurements of K_{D} values are preferably made with standardized solutions of protein and a standardized buffer, such as PBS.

Binding of any molecule to another is governed by two forces, namely the association rate (kₒₙ) and the dissociation rate (k_{off}). The affinity of any binder [B] to a target [T] can then be expressed by the equilibrium dissociation constant K_{D}, which is the quotient of k_{off}/kₒₙ.

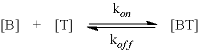

kₒₙ is a second-order rate constant of the binding reaction, with the unit M⁻¹s⁻¹, whereas the dissociation reaction k_{off} is a first-order rate constant with the unit s⁻¹. From this it becomes clear that the association reaction depends on the concentration of the reactants, whereas the dissociation is independent of the concentration, following a simple exponential decay function.

A variety of methods of measuring binding affinity are known in the art, any of which can be used for purposes of the present invention. For example, as exemplified herein, the binding affinity of a particular binding moiety to a drug molecule target can be expressed as K_{D} value, which refers to the dissociation constant of the binding moiety and the drug molecule target. K_{D} is the ratio of the rate of dissociation, also called the “off-rate (k_{off})”, to the association rate, or “on-rate (kₒₙ)”. Thus, K_{D} equals k_{off}/kₒₙ and is expressed as a molar concentration (M), and the smaller the k_{D}, the stronger the affinity of binding. K_{D} values can be determined using any suitable method. One exemplary method for measuring K_{D} is surface plasmon resonance (SPR) (see, e.g., Nguyen et al. Sensors (Basel). 2015 May 5; 15 (5): 10481-510). K_{D} value may be measured by SPR using a biosensor system such as a BIACORE® system. BIAcore kinetic analysis comprises, e.g., analysing the binding and dissociation of an antigen from chips with immobilized molecules (e.g., molecules comprising epitope binding domains), on their surface. Another method for determining the K_{D} of a protein is by using Bio-Layer Interferometry (see, e.g., Shah et al. J Vis Exp. 2014; (84): 51383). A K_{D} value may be measured using OCTET® technology (Octet QKe system, ForteBio). Alternatively, or in addition, a KinExAR (Kinetic Exclusion Assay) assay, available from Sapidyne Instruments (Boise, Id.) can also be used. Any method suitable for assessing the binding affinity between two binding partners is encompassed herein. Surface plasmon resonance (SPR) is particularly preferred. Most preferably, the K_{D} values are determined in PBS and by SPR.

A typical and preferred determination of dissociation constants (K_{D}) of the inventive recombinant binding proteins with binding specificity for CD47 by Surface Plasmon Resonance (SPR) analysis is described in Example 3.

The term “binding agent” refers to any molecule capable of specifically binding a target molecule. Binding agents include, for example, antibodies, antibody fragments, aptamers, peptides (e.g., Williams et al., J Biol Chem 266:5182-5190 (1991)), antibody mimics, repeat proteins, e.g. designed ankyrin repeat proteins, receptor proteins and any other naturally occurring interaction partners of the target molecule, and can comprise natural proteins and proteins modified or genetically engineered, e.g., to include non-natural residues and/or to lack natural residues.

The term “about” means the mentioned value+/−20%; for example “about 50” shall mean 40 to 60.

The term “PBS” means a phosphate buffered water solution containing 137 mM NaCl, 10 mM phosphate and 2.7 mM KCl and having a pH of 7.4.

For the purpose of this disclosure, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotides or amino acids at each position are then compared. When a position in the first sequence is occupied by the same amino acid or nucleotide as the corresponding position in the second sequence, then the amino acids or nucleotides are identical at that position. The percent identity between the two sequences is a function of the number of identical positions shared by the sequences (i.e., % identity=number of identical positions/total number of positions in the reference sequence×100).

Typically, the sequence comparison is carried out over the length of the reference sequence. For example, if the user wished to determine whether a given (“test”) sequence is 95% identical to SEQ ID NO: 1, SEQ ID NO: 1 would be the reference sequence. To assess whether a sequence is at least 80% identical to SEQ ID NO: 1 (an example of a reference sequence), the skilled person would carry out an alignment over the length of SEQ ID NO: 1, and identify how many positions in the test sequence were identical to those of SEQ ID NO: 1. If at least 80% of the positions are identical, the test sequence is at least 80% identical to SEQ ID NO: 1. If the sequence is shorter than SEQ ID NO: 1, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. For example, the percent identity between two amino acid or nucleic acid sequences can be determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. One suitable set of parameters are a Blossum 62 scoring matrix with a gap penalty of 12, a gap extend penalty of 4, and a frameshift gap penalty of 5. The term “mouse serum albumin” refers to UniProt accession number P07724, the term “cynomolgus monkey serum albumin” (i.e. Macaca fascicularis) refers to UniProt accession number A2V9Z4, and the term “human serum albumin” refers to UniProt accession number P02768.

Preferably, clearance, and/or exposure, and/or terminal half-life are assessed in a mammal, more preferably mouse and/or cynomolgus monkey, more preferably cynomolgus monkey. Clearance, and/or exposure, and/or terminal half-life may be assessed as described in Example 3. Preferably, when measuring the clearance, and/or exposure, and/or terminal half-life in mouse, the evaluation is done considering the data up to 48 h post-injection. More preferably, the evaluation of terminal half-life in mouse is calculated from 24 h to 48 h. Preferably, when measuring the clearance, and/or exposure, and/or terminal half-life in cynomolgus monkey, the evaluation is done considering the data up to day 7 post-injection. More preferably, the evaluation of terminal half-life in cynomolgus monkey is calculated from day 1 to day 5. The person skilled in the art further is able to identify effects such as target-mediated clearance and consider them when calculating the terminal half-life. The term “terminal half-life” of a drug such as a recombinant binding protein of the invention refers to the time required to reach half the plasma concentration of the drug applied to a mammal after reaching pseudo-equilibrium (for example calculated from 24 hours to 48 hours in mouse or calculated from day 1 to day 5 in cynomolgus monkey). Terminal half-life is not defined as the time required to eliminate half the dose of the drug administered to the mammal. The term terminal half-life is known to the person skilled in the art. Preferably, pharmacokinetic comparison is done at any dose, more preferably at equivalent dose (i.e. same mg/kg dose) or equimolar dose (i.e. same mol/kg dose), more preferably at equimolar dose (i.e. same mol/kg dose). It is understood by the person skilled in the art that equivalent and/or equimolar dosing in animals is subject to experimental dose variations of at least 20%, 30%, 40%, 50%, 60%, 70%, 80%, 90%, or 100%. Preferably, a dose used for pharmacokinetic measurement is selected from 0.001 to 1000 mg/kg, more preferably 0.01 to 100 mg/kg, more preferably 0.1 to 50 mg/kg, more preferably 0.5 to 10 mg/kg.

The term “tumor-localized activation of immune cells” means that immune cells are activated preferentially in tumor tissue as compared to a non-tumor tissue.

The term “treat” and “treatment” refer to both therapeutic treatment and prophylactic or preventive measures, wherein the object is to prevent or slow down an undesired physiological change or disorder. For purpose of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

Furthermore, the term “peptide” also encompasses peptides modified by, e.g, glycosylation, and proteins comprising two or more polypeptide chains, each of length of 4 to 600 amino acids long, cross-linked by, e.g., disulfide bonds, such as, e.g., insulin and immunoglobulins. The term “chemical or biochemical agent” is intended to include any naturally occurring or synthetic compound that may be administered to a recipient.

The term “medical condition” (or disorder or disease) includes autoimmune disorders, inflammatory disorders, retinopathies (particularly proliferative retinopathies), neurodegenerative disorders, infections, metabolic diseases, and neoplastic diseases. Any of the recombinant binding proteins described herein may be used for the preparation of a medicament for the treatment of such a disorder, particularly a disorder selected from the group comprising: an autoimmune disorder, an inflammatory disorder, an immune disorder, and a neoplastic disease. A “medical condition” may be one that is characterized by inappropriate cell proliferation. A medical condition may be a hyperproliferative condition. The invention particularly relates to a method of treating a medical condition, the method comprising the step of administering, to a subject in need of such treatment, a therapeutically effective amount of a recombinant binding protein or said pharmaceutical composition of the invention. In a preferred embodiment said medical condition is a neoplastic disease. The term “neoplastic disease”, as used herein, refers to an abnormal state or condition of cells or tissue characterized by rapidly proliferating cell growth or neoplasm. In one embodiment said medical condition is a malignant neoplastic disease. In one embodiment said medical condition is cancer. The term “therapeutically effective amount” means an amount that is sufficient to produce a desired effect on a subject.

The term “antibody” means not only intact antibody molecules, but also any fragments and variants of antibody molecules that retain immunogen-binding ability. Such fragments and variants are also known in the art and are regularly employed both in vitro and in vivo. Accordingly, the term “antibody” encompasses intact immunoglobulin molecules, antibody fragments such as, e.g., Fab, Fab′, F(ab′)₂, and single chain V region fragments (scFv), bispecific antibodies, chimeric antibodies, antibody fusion polypeptides, and unconventional antibodies.

Alternative scaffolds include any polypeptides or proteins comprising a binding domain that is capable of binding a target and that is not derived from an antibody or immunoglobulin molecule. The binding domain of alternative scaffolds may comprise or may be derived from a variety of different polypeptide or protein structures. Alternative scaffolds include, but are not limited to, adnectins (monobodies), affibodies, affilins, affimers and aptamers, affitins, alphabodies, anticalins, armadillo repeat protein-based scaffolds, atrimers, avimers, ankyrin repeat protein-based scaffolds (such as DARPin proteins), fynomers, knottins, and Kunitz domain peptides. Alternative scaffolds are described, e.g., in Yu et al., Annu Rev Anal Chem (Palo Alto Calif). 2017 Jun. 12; 10 (1): 293-320. doi: 10.1146/annurevanchem-061516-045205.

The terms “cancer” and “cancerous” are used herein to refer to or describe the physiological condition in mammals that is typically characterized by unregulated cell growth. Cancer encompasses solid tumors and liquid tumors, as well as primary tumors and metastases. A “tumor” comprises one or more cancerous cells. Solid tumors typically also comprise tumor stroma. Examples of cancer include, but are not limited to, primary and metastatic carcinoma, lymphoma, blastoma, sarcoma, and leukemia, and any other epithelial and lymphoid malignancies. More particular examples of such cancers include brain cancer, bladder cancer, breast cancer, ovarian cancer, clear cell kidney cancer, head/neck squamous cell carcinoma, lung adenocarcinoma, lung squamous cell carcinoma, malignant melanoma, non-small-cell lung cancer (NSCLC), ovarian cancer, pancreatic cancer, prostate cancer, renal cell carcinoma, small-cell lung cancer (SCLC), breast cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), Squamous Cell Carcinoma of the Head and Neck (SCCHN), chronic myelogenous leukemia (CML), small lymphocytic lymphoma (SLL), malignant mesothelioma, colorectal cancer, or gastric cancer.

### EXAMPLES

Starting materials and reagents disclosed below are known to those skilled in the art, are commercially available and/or can be prepared using well-known techniques.

### Materials

Chemicals were purchased from Sigma-Aldrich (USA). Oligonucleotides were from Microsynth (Switzerland). Unless stated otherwise, DNA polymerases, restriction enzymes and buffers were from New England Biolabs (USA) or Fermentas/Thermo Fisher Scientific (USA). Inducible E. coli expression strains were used for cloning and protein production, e.g. E. coli XL1-blue (Stratagene, USA) or BL21 (Novagen, USA).

### Molecular Biology

Unless stated otherwise, methods are performed according to known protocols (see, e.g., Sambrook J., Fritsch E. F. and Maniatis T., Molecular Cloning: A Laboratory Manual, Cold Spring Harbor Laboratory 1989, New York).

### Designed Ankyrin Repeat Protein Libraries

Methods to generate designed ankyrin repeat protein libraries have been described, e.g. in U.S. Pat. No. 7,417,130; Binz et al. 2003, loc. cit.; Binz et al. 2004, loc. cit. By such methods designed ankyrin repeat protein libraries having randomized ankyrin repeat modules and/or randomized capping modules can be constructed. For example, such libraries could accordingly be assembled based on a fixed N-terminal capping module (e.g. the N-terminal capping module of any of SEQ ID NOs: 22 to 36) or a randomized N-terminal capping module (e.g. the N-terminal capping module of any of SEQ ID NOs: 82 to 87), and a fixed C-terminal capping module (e.g. the C-terminal capping module of SEQ ID NOs: 37 to 60) or a randomized C-terminal capping module (e.g. the Ce-terminal capping module of SEQ ID NO: 88). Preferably, such libraries are assembled to not have any of the amino acids C, G, M, N (in front of a G residue) and P at randomized positions of repeat or capping modules.

Furthermore, such randomized modules in such libraries may comprise additional polypeptide loop insertions with randomized amino acid positions. Examples of such polypeptide loop insertions are complement determining region (CDR) loop libraries of antibodies or de novo generated peptide libraries. For example, such a loop insertion could be designed using the structure of the N-terminal ankyrin repeat domain of human ribonuclease L (Tanaka, N., Nakanishi, M, Kusakabe, Y, Goto, Y., Kitade, Y, Nakamura, K. T., EMBO J. 23 (30), 3929-3938, 2004) as guidance. In analogy to this ankyrin repeat domain where ten amino acids are inserted in the beta-turn present close to the boarder of two ankyrin repeats, ankyrin repeat proteins libraries may contain randomized loops (with fixed and randomized positions) of variable length (e.g. 1 to 20 amino acids) inserted in one or more beta-turns of an ankyrin repeat domain.

Any such N-terminal capping module of an ankyrin repeat protein library preferably possesses the RILLAA, RILLKA or RELLKA motif (e.g. present from position 21 to 26 in SEQ ID NO: 1) and any such C-terminal capping module of an ankyrin repeat protein library preferably possesses the KLN, KLA or KAA motif (e.g. present at the last three amino acids in SEQ ID NO: 1). SEQ ID NOS: 22 to 36 provide examples of N-terminal capping modules comprising the RILLAA, RILLKA or RELLKA motif, and SEQ ID NOs: 37 to 60 provide examples of C-terminal capping modules comprising the KLN, KLA or KAA motif.

The design of such an ankyrin repeat protein library may be guided by known structures of an ankyrin repeat domain interacting with a target. Examples of such structures, identified by their Protein Data Bank (PDB) unique accession or identification codes (PDB-IDs), are 1WDY, 3V31, 3V30, 3V2X, 3V2O, 3UXG, 3TWQ-3TWX, 1N11, 1S70 and 2ZGD.

Examples of designed ankyrin repeat protein libraries, such as N2C and N3C designed ankyrin repeat protein libraries, have been described (U.S. Pat. No. 7,417,130; Binz et al. 2003, loc. cit.; Binz et al. 2004, loc. cit.). The digit in N2C and N3C describes the number of randomized repeat modules present between the N-terminal and C-terminal capping modules.

The nomenclature used to define the positions inside the repeat units and modules is based on Binz et al. 2004, loc. cit. with the modification that borders of the ankyrin repeat modules and ankyrin repeat units are shifted by one amino acid position. For example, position 1 of an ankyrin repeat module of Binz et al. 2004 (loc. cit.) corresponds to position 2 of an ankyrin repeat module of the current disclosure and consequently position 33 of an ankyrin repeat module of Binz et al. 2004, loc. cit. corresponds to position 1 of a following ankyrin repeat module of the current disclosure.

All the DNA sequences were confirmed by sequencing, and the calculated molecular weight of selected proteins was confirmed by mass spectrometry.

### Example 1: Selection of Binding Proteins Comprising an Ankyrin Repeat Domain with Binding Specificity for CD47

Using ribosome display (Hanes, J. and Pluckthun, A., PNAS 94, 4937-42, 1997), several ankyrin repeat proteins with binding specificity for human CD47 were selected from DARPin libraries similar as described by Binz et al. 2004 (loc. cit.). The binding of the selected clones towards recombinant human CD47 target protein was assessed by crude extract Homogeneous Time Resolved Fluorescence (HTRF), indicating that hundreds of human CD47-specific binding proteins were successfully selected. For example, the ankyrin repeat domains of SEQ ID NOs: 1 to 8 constitute amino acid sequences of selected binding proteins comprising an ankyrin repeat domain with binding specificity for CD47. Individual ankyrin repeat modules from such ankyrin repeat domains with binding specificity for CD47 are provided, e.g., in SEQ ID NOs: 9 to 21.

### Human Recombinant CD47 Target Preparation

The target format chosen was based on single chain format, consisting of the extracellular domain of human CD47 protein (Uniprot ID: Q08722, residues 19 to 141, SEQ ID NO: 67; purchased from BPS) linked to an Fc domain and a C-terminal Avi-tag for site-directed biotinylation. It was expressed in Human embryonic kidney 293 cells (HEK293) cells. A batch of biotinylated and a batch of non-biotinylated protein were purchased. The final material was formulated in 8 mM phosphate at pH 7.4, 110 mM NaCl, 2.2 mM KCl and 20% glycerol.

### Selection of CD47-Specific Ankyrin Repeat Proteins by Ribosome Display

The selection of CD47-specific ankyrin repeat proteins was performed by ribosome display (Hanes and Plückthun, loc. cit.) using part of the extracellular domain of CD47 (SEQ ID NO: 67) as target protein, libraries of ankyrin repeat proteins as described above, and established protocols (see, e.g., Zahnd, C., Amstutz, P. and Pluckthun, A., Nat. Methods 4, 69-79, 2007). The number of reverse transcription (RT)-PCR cycles after each selection round was constantly thirty. The first four rounds of selection employed standard ribosome display selection, using decreasing target concentration (400 nM, 100 nM, 25 nM and 5 nM, respectively), followed by a 5^{th} off rate round using a target concentration of 1 nM and a competitor benchmark anti-CD47 antibody at 1000-fold higher concentration in order to select high affinity binders, and a 6^{th} recovery round at a target concentration of 5 nM.

### Selected Clones Show Binding to CD47 Target (Shown by HTRF and SIRPa/Ligand Competition)

In a first screening step, the pools from the ribosome display were expressed in E. coli cells in 96 well plates. 12 plates of DARPin proteins from ribosome display round 4 and 14 plates of DARPin proteins from round 6 were expressed in E. coli cells with a Flag-tag and a His-tag. Crude extracts thereof were prepared to test binding of the Flag-tagged DARPin proteins to human CD47 recombinant protein in an HTRF assay. The crude extracts were diluted 1:1600 in PBS-TB (PBS supplemented with 0.2% (w/v) BSA and 0.1% Tween20, pH 7.4) and used at 1:6400 dilution (final) in the assay. Binding was performed against 6 nM (final concentration) of human biotinylated CD47-Fc. FRET donor (Streptavidin-Tb) and acceptor (MAb Anti Flag M2-d2) conjugate (Cisbio) were used at a 1:400 dilution (final) in a well of a 384-well plate and incubated for 60 minutes at room temperature (RT). The HTRF was read-out on a Tecan M1000 using a 340 nm excitation wavelength and a 665±10 nm emission filter.

Four plates of binders were again expressed in E. coli cells and purified on 96 well plates using their His-tag according to standard protocols. The proteins were monomeric and soluble when concentrated to 10 mg/ml in TBS pH 8.0 (50 mM Tris, 500 mM NaCl). The following panel of eight designed ankyrin repeat proteins with binding specificity for human CD47 were selected:
DARPin protein #1 (SEQ ID NO:1 with a His-tag (SEQ ID NO: 61) fused to its N terminus);
DARPin protein #2 (SEQ ID NO:2 with a His-tag (SEQ ID NO: 61) fused to its N terminus);
DARPin protein #3 (SEQ ID NO:3 with a His-tag (SEQ ID NO: 61) fused to its N terminus);
DARPin protein #4 (SEQ ID NO:4 with a His-tag (SEQ ID NO: 61) fused to its N terminus);
DARPin protein #5 (SEQ ID NO:5 with a His-tag (SEQ ID NO: 61) fused to its N terminus);
DARPin protein #6 (SEQ ID NO:6 with a His-tag (SEQ ID NO: 61) fused to its N terminus);
DARPin protein #7 (SEQ ID NO:7 with a His-tag (SEQ ID NO: 61) fused to its N terminus);
DARPin protein #8 (SEQ ID NO:8 with a His-tag (SEQ ID NO: 61) fused to its N terminus).

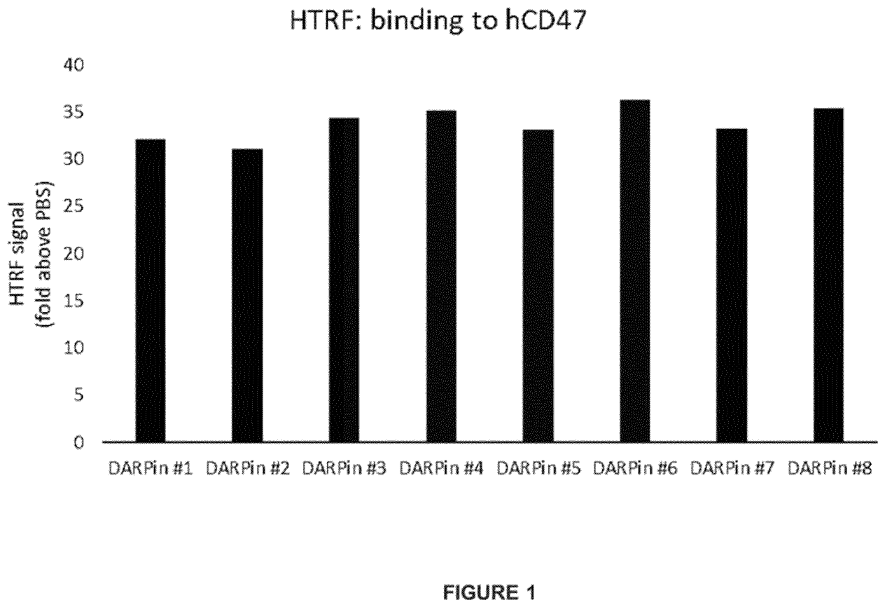

Binding of the purified designed ankyrin repeat proteins in HTRF was confirmed with 40 nM of each designed ankyrin repeat protein and 10 nM of human biotinylated CD47-Fc (see FIG. 1).

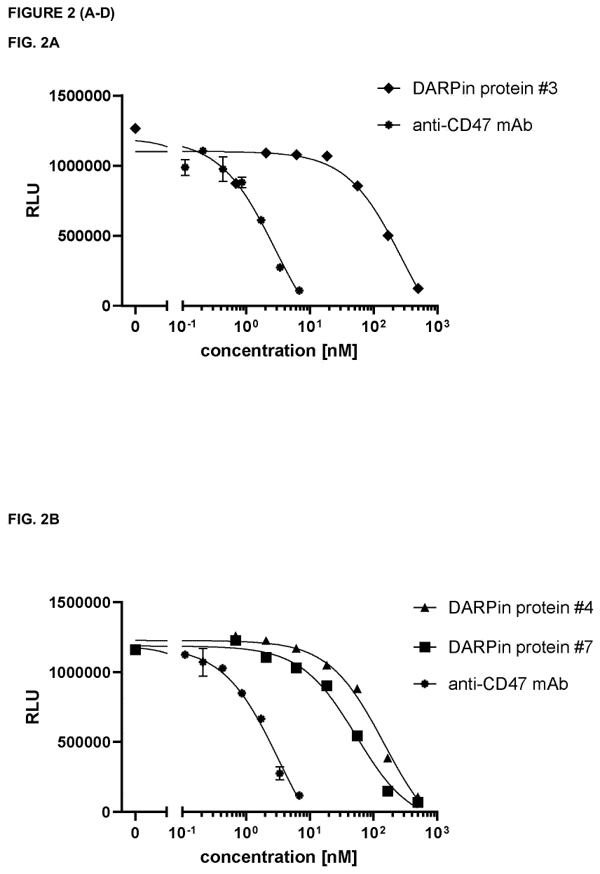

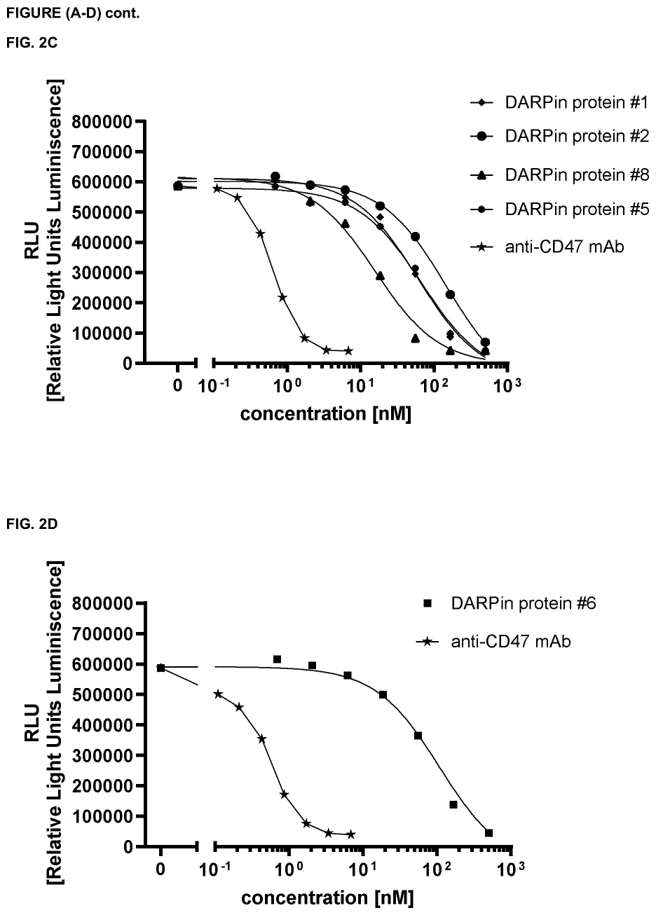

The eight selected designed ankyrin repeat proteins binding to human CD47 were tested for SIRP1α competition using the PathHunter Jurkat SIRPα Signaling Bioassay Kit purchased from Eurofins DiscoverX products LLC. Ligand engagement through co-culture of Jurkat CD47 presenting cells and Jurkat SIRP1α signaling cells results in phosphorylation and intracellular pathway activation leading to a chemiluminescence signal. This signal can be inhibited in the presence of an anti-CD47 antibody or anti-SIRPαantibody disrupting ligand-receptor interaction. As can be seen in FIGS. 2 (A-D), all eight designed ankyrin repeat proteins were able to inhibit ligand interaction. IC50 values of DARPin proteins #1 to 8 obtained in this assay are shown in Table 2.

TABLE 2
Ligand inhibition (IC50 values)
for DARPins #1 to 8

| DARPin protein | IC50 (nM) |
|---|---|
| #1 | 60.4 |
| #2 | 146 |
| #3 | 278.4 |
| #4 | 146.7 |
| #5 | 67.8 |
| #6 | 90.5 |
| #7 | 53.5 |
| #8 | 124 |

### Cell Binding by Selected CD47-Binding Designed Ankyrin Repeat Proteins.

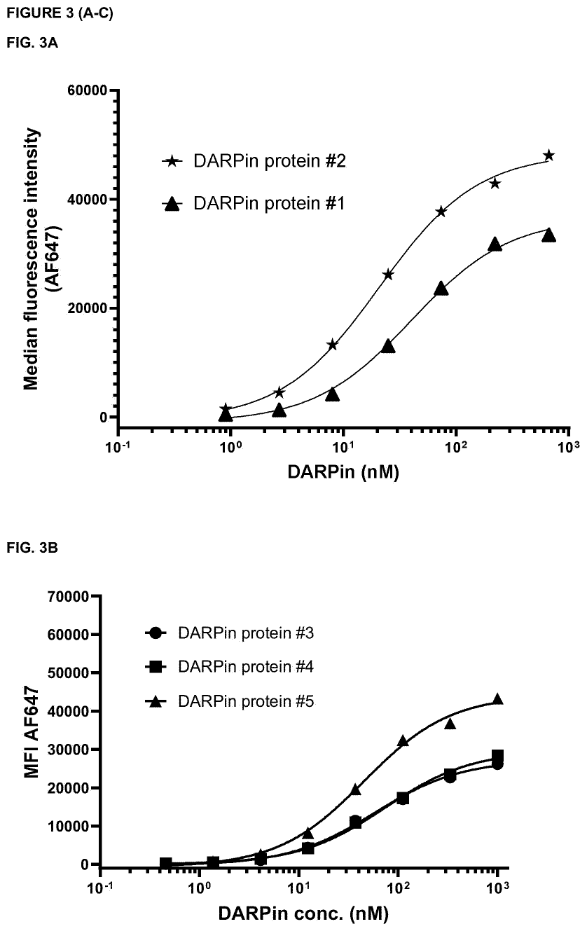

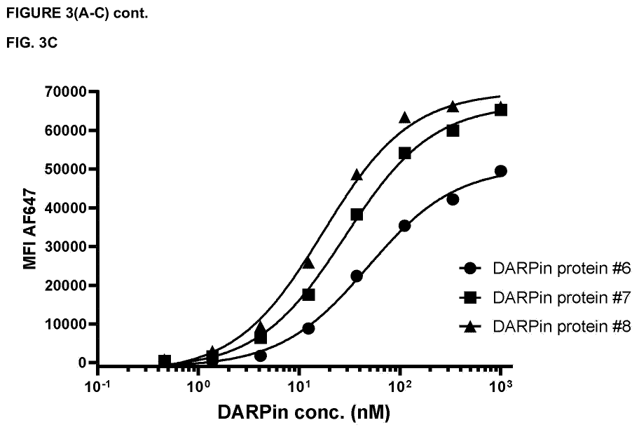

The above selected designed ankyrin repeat proteins were further tested for CD47 specific cell binding. CD47 binding was confirmed by a cell binding assay using a Raji cell line (purchased from ATCC), a cancer cell line expressing CD47. In brief, the CD47 expressing cells were washed 1× with 500 μl PBS, spun at 350 g for 5 minutes and the supernatant was discarded. Then, a titration of selected CD47 designed ankyrin repeat proteins diluted in FACS buffer was added to the pellet of cells, resuspended and incubated for 30 minutes at 4° C. Cells were washed twice with 200 μl cold PBS, spun at 350 g for 5 minutes and the supernatant was discarded. 50 μl of anti-DARPin antibody AF-647 with live/dead aqua (1:3000), was added to the cells and incubated for 30 mins at 4° C., in the dark. Then the cells were washed twice in 200 μl ice cold PBS and spun for 4 min at 400 g. 50 μl of cell fix was added and the cells were incubated for 60 min at 4° C., then they were washed with PBS/2 mM EDTA, spun for 5 min at 500 g and resuspended in 200 μl of PBS/2 mM EDTA. Flow cytometry analysis was performed using a Attune Nxt Flow Cytometer. All tested proteins showed binding to the target on cells as shown in FIG. 3 (A-C) and Table 3.

TABLE 3
Cell binding of selected CD47-binding
designed ankyrin repeat proteins

| DARPin protein | EC50 (nM) |
|---|---|
| #1 | 40.4 |
| #2 | 20.9 |
| #3 | 58.4 |
| #4 | 74.7 |
| #5 | 45.5 |
| #6 | 48 |
| #7 | 28.5 |
| #8 | 17.2 |

### Example 2: Stability Assessment of Exemplary CD47-Specific Ankyrin Repeat Proteins

Assessment of exemplary CD47-specific ankyrin repeat proteins using SDS-PAGE and Size-Exclusion Chromatography (SEC)

The above selected CD47-specific designed ankyrin repeat proteins were analysed in-depth to assess their biophysical properties. In brief, purified CD47-specific ankyrin repeat proteins DARPin #1 (SEQ ID NO:1), DARPin #2 (SEQ ID NO: 2), DARPin #3 (SEQ ID NO: 3), DARPin #4 (SEQ ID NO: 4), DARPin #5 (SEQ ID NO: 5), DARPin #6 (SEQ ID NO: 6), DARPin #7 (SEQ ID NO: 7) and DARPin #8 (SEQ ID NO: 8) in PBS, pH7.4 were aliquoted into sterile glass vials (Schmidlin: LPP 11 09 0620) and stressed by incubation at 60° C. for 1 week. The applied stress conditions allow a prediction of the biophysical properties after 2 years storage at 4° C. For each CD47-specific ankyrin repeat protein, an aliquot was stored at −70° C. as a reference. The reference and the heat-stressed samples were exposed to one freeze-thaw cycle before analysis. The samples were evaluated for multimerization, aggregation and fragmentation by SDS-PAGE and analytic SEC. Stressed samples were then compared to the respective reference samples. No colour change, turbidity or precipitation was detected by visual inspection. Furthermore, no significant degradation or aggregation was detected. The results of the experiments demonstrated that samples were stable in the applied stress conditions.

### Thermal Stability Assessment of Exemplary CD47-Specific Ankyrin Repeat Proteins Using Circular Dichroism (CD) Spectroscopy

The same CD47-specific ankyrin repeat proteins were also assessed for their thermal stability and unfolding/refolding propensity using a Jasco J-815 spectrophotometer (non-stressed proteins only). The Tm (melting temperature) of the selected proteins is determined by CD as a parameter for thermal stability. The ellipticity was recorded at 222 nM and a temperature range from 20° C. to 90° C. was applied followed by reverse scan to record the refolding behavior. The Tm of the selected CD47-specific ankyrin repeat proteins is the midpoint of the protein unfolding. Spectra from 190-250 nm were recorded before and after the temperature scan.

Samples were prepared at a concentration of 2 μM in PBS, pH7.4. As it can be seen in Table 4, for all tested proteins a Tm higher than 80° C. was obtained.

TABLE 4
Tₘ (melting temperature) of eight exemplary
CD47-specific ankyrin repeat proteins

| DARPin | SEQ ID | |
|---|---|---|
| protein # | NO | Tm ° C. |
| #1 | 1 | Above 80° C. |
| #2 | 2 | Above 80° C. |
| #3 | 3 | Above 85° C. |
| #4 | 4 | Above 80° C. |
| #5 | 5 | Above 90° C. |
| #6 | 6 | Above 85° C. |
| #7 | 7 | Above 85° C. |
| #8 | 8 | Above 90° C. |

### Example 3: Determination of Dissociation Constants (K_{D}) of Ankyrin Repeat Proteins with Binding Specificity for Human CD47 by Surface Plasmon Resonance (SPR) Analysis

The binding affinities of eight purified ankyrin repeat proteins to biotinylated recombinant human CD47-Fc target were analyzed using a ProteOn XPR 36 instrument (NAHLC200M, Xantec ProteOn Sensor Chip) and the measurement was performed according to standard procedures. For that, SEQ ID NO: 1 to SEQ ID NO: 8 were subcloned into derivatives of the pQE30 (Qiagen) expression vector, containing an N-terminal His-tag, and expressed and purified as described above (yielding DARPin protein #1, DARPin protein #2, DARPin protein #3, DARPin protein #4, DARPin protein #5, DARPin protein #6, DARPin protein #7 and DARPin protein #8).

Briefly, biotinylated human CD47-Fc target was diluted in PBST (PBS, pH 7.4 containing 0.005% Tween 20®) and coated on an NAHLC200M chip (BioRad) to a level of around 350 and 800 resonance units (RU). The interaction of ankyrin repeat protein and human CD47 was then measured by injecting 200 μl running buffer (PBS, pH 7.4 containing 0.005% Tween 20®) containing serial dilutions of ankyrin repeat proteins covering a concentration range between 16.7 nM, 5.6 nM, 1.85 nM and 0.62 nM for multi-trace SPR measurements, followed by a running buffer flow for at least 20 minutes at a constant flow rate of 100 μl/min (off-rate measurement). The regeneration was performed using 30 μl of 10 mM Glycine pH 2.5. The signals (i.e. resonance unit (RU) values) of an empty lane (no target coated) and a reference injection (i.e. injection of running buffer only) were subtracted from the RU traces obtained after injection of ankyrin repeat protein (double-referencing). Binding parameters (K_{D}, on-rate, off-rate) against CD47 were determined for the constructs as shown in Table 4.

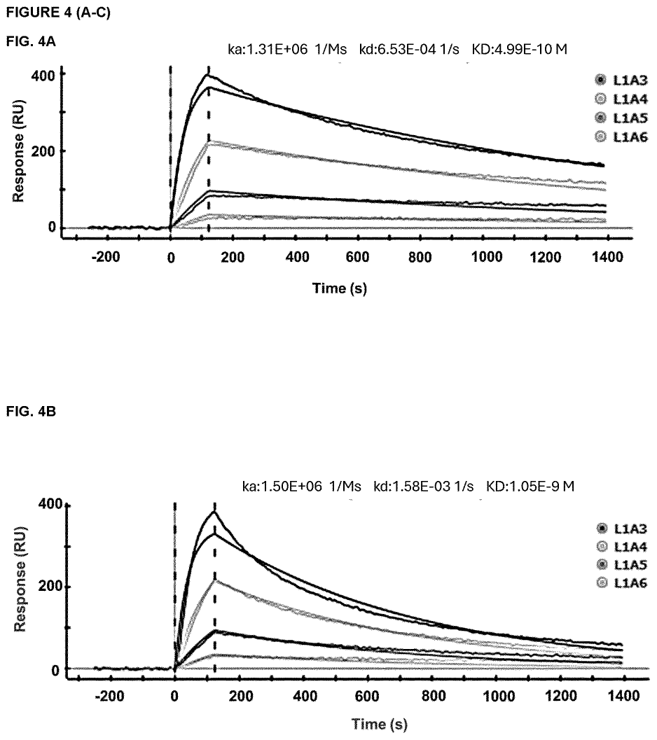

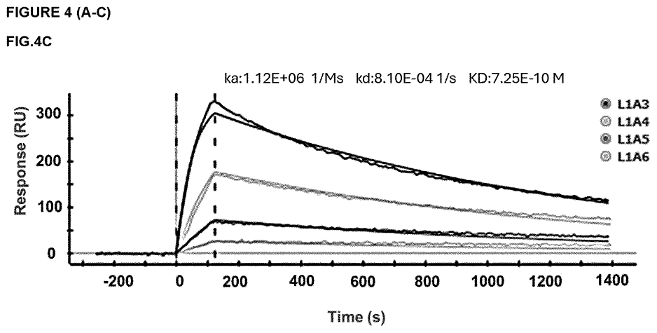

As representative examples, FIG. 4 (A-C) shows SPR traces obtained for DARPin molecules #2, 4 and 7. Dissociation constants (K_{D}) were calculated from the estimated on- and off-rates using standard procedures. K_{D} values of the binding interactions of selected ankyrin repeat proteins with human CD47 were determined to be in the range of 0.1 to 0.8 nM (see Table 5).

TABLE 5
Binding parameters for ankyrin repeat proteins with binding
specificity for human CD47 by Surface Plasmon Resonance (SPR)

| DARPin protein # | KD [nM] | Kon [1/Ms] | Koff [1/s] |
|---|---|---|---|
| DARPin protein # 1 | 0.328 | 1.7E+06 | 5.5E−04 |
| DARPin protein #2 | 0.813 | 1.9E+06 | 1.5E−03 |
| DARPin protein #3 | 0.562 | 1.4E+06 | 7.7E−04 |
| DARPin protein #4 | 0.244 | 2.5E+06 | 6.0E−04 |
| DARPin protein #5 | 0.552 | 7.6E+05 | 4.2E−04 |
| DARPin protein #6 | 0.240 | 1.4E+06 | 3.3E−04 |
| DARPin protein #7 | 0.352 | 8.5E+05 | 3.0E−04 |
| DARPin protein #8 | 0.102 | 1.4E+06 | 1.4E−04 |

### Example 4: Pharmacokinetic Analysis of CD47 Specific Ankyrin Repeat Proteins in Female BALB/c Mice

In order to determine whether a CD47-specific ankyrin repeat domain can have an appropriate serum half-life in vivo, the pharmacokinetic profiles of the DARPin proteins of SEQ ID NOs: 4 to 8 were analyzed in mice. DARPin sequences were subcloned and expressed as described above into derivatives of the pQE30 (Qiagen) expression vector. The constructs contained an N-terminal His-tag, followed by an HSA (human serum albumin) binding ankyrin repeat domain for half-life extension (SEQ ID NO: 63), and then followed by one of the CD47 specific binding domains. For example, expression vectors encoding the following ankyrin repeat proteins were constructed:
DARPin protein #9 comprising SEQ ID NO: 68;
DARPin protein #10 comprising SEQ ID NO: 69;
DARPin protein #11 comprising SEQ ID NO: 70;
DARPin protein #12 comprising SEQ ID NO: 71;
DARPin protein #13 comprising SEQ ID NO: 72.

### In Vivo Administration and Sample Collection

DARPin protein #9, DARPin protein #10, DARPin protein #11, DARPin protein #12 and DARPin protein #13 were administered as a single intravenous bolus injection into the tail vein of six mice for each ankyrin repeat fusion protein. The target dose level was 1 mg/kg with an application volume of 5 mL/kg. Ankyrin repeat fusion proteins were formulated in phosphate-buffered saline (PBS) solution.

Mice were split into two groups with equal numbers of animals. Four serum samples were collected from each mouse. Blood samples for pharmacokinetic investigations were collected from the saphenous vein at 5 min, 6 h, 24 h, 48 h, 76 h, 96 h and 168 h post dosing. Blood was kept at room temperature to allow clotting followed by centrifugation and collection of serum.

### Bioanalytics by ELISA to Measure Ankyrin Repeat Proteins in Serum Samples

100 μl per well of 10 nM polyclonal goat anti-rabbit IgG antibody (Ab18) in PBS was coated onto a NUNC Maxisorb ELISA plate overnight at 4° C. After washing with 300 μl PBST (PBS supplemented with 0.1% Tween20) per well three times, the wells were blocked with 300 μl PBST supplemented with 0.25% Casein (PBST-C) for 1 h at room temperature (RT) on a Heidolph Titramax 1000 shaker (450 rpm). Plates were washed as described above. 100 μl 5 nmol/L rabbit anti-DARPin 1-1-1 antibody in PBST-C was added and the plates were incubated at RT with orbital shaking (450 rpm) for 1 h. Plates were washed as described above.

100 μl of diluted serum samples (1:20-1:312500 in 1:5 dilution steps) or ankyrin repeat protein standard curve samples (0 and 50-0.0008 nmol/L in 1:3 dilution steps) were applied for 2 h, at RT, shaking at 450 rpm. Plates were washed as described above.

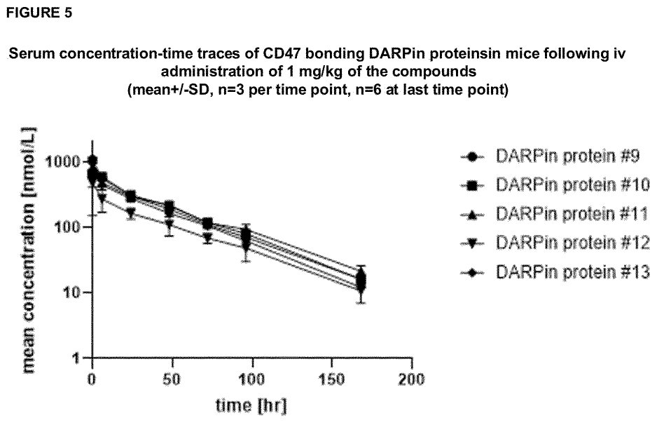

Wells were then incubated with 100 μl murine anti-RGS-His-HRP IgG (Ab06, 1:2000 in PBST-C) and incubated for 1 h, at RT, 450 rpm. Plates were washed as described above. The ELISA was developed using 100 μl/well TMB substrate solution for 10 minutes and stopped by the addition of 100 μl 1 mol/L H₂SO₄. The difference between the absorbance at 450 nm and the absorbance at 620 nm was calculated. Samples were measured in duplicate on two different plates. FIG. 5 shows the serum concentrations of DARPin protein #9, DARPin protein #10, DARPin protein #11, DARPin protein #12 and DARPin protein #13 as a function of time after the single intravenous administration into mice. The traces indicate mono-exponential elimination of the compounds.

### Pharmacokinetic Analysis

Pharmacokinetic data analysis was performed using Version 8.3 of the WinNonlin program as part of Phoenix 64, Pharsight, North Carolina. Calculation of the pharmacokinetic parameters based on the mean concentration-time data of the animals dosed via intravenous bolus injection was performed with non-compartmental analysis (NCA model 200-202, IV bolus, linear trapezoidal linear interpolation). The following pharmacokinetic parameters were calculated:

$${AUC}_{inf} , {AUC}_{last} , AUC\_ \% extrapol , {C}_{max} , {T}_{max} , CI\_pred , Vss\_pred , {t}_{1 / 2}$$

Maximum serum concentrations (Cₘₐₓ) and the times of their occurrence (Tₘₐₓ) were obtained directly from the serum concentration-time profiles. The area under the serum concentration-time curve (AUC_{inf}) was determined by the linear trapezoidal formula up to the last sampling point (Tₗₐₛₜ) and extrapolation to infinity assuming mono-exponential decrease of the terminal phase. The extrapolation up to infinity was performed using Cₗₐₛₜ/λz, where λz denotes the terminal rate constant estimated by log linear regression and Cₗₐₛₜ denotes the concentration estimated at Tₗₐₛₜ by means of the terminal log-linear regression. Total serum clearance (CI_pred) and the apparent terminal half-life were calculated as follows: Cl_pred=i.v. dose/AUC_{inf} and t_{1/2}=ln2/λz. The steady-state volume of distribution Vss was determined by: Vss=i.v. dose. AUMC_{inf}/(AUC_{inf}) 2. AUMC_{inf} denotes the total area under the first moment of drug concentration-time curve extrapolated to infinity using the same extrapolation procedure as described for calculation of AUC_{inf}. TO calculate PK parameters based on concentrations given in nmol/L dose values given as mg/kg were converted to nmol/kg by using the molecular weight of the ankyrin repeat proteins. Table 5 shows the summary of pharmacokinetic characteristics of the five tested ankyrin repeat proteins DARPin protein #9, DARPin protein #10, DARPin protein #11, DARPin protein #12 and DARPin protein #13 following single intravenous administration of 1 mg/kg.

TABLE 6
Pharmacokinetic parameters for five exemplary HSA/CD47 specific ankyrin repeat proteins

| | | DARPin | DARPin | DARPin | DARPin | DARPin |
|---|---|---|---|---|---|---|
| parameter | unit | protein #9 | protein #10 | protein #11 | protein #12 | protein #13 |
| AUCINF_pred | h*(nmol/L) | 28572 | 30014 | 29223 | 15888 | 24734 |
| AUClast | h*(nmol/L) | 27768 | 29204 | 27991 | 15287 | 24152 |
| Cmax | nmol/L | 1088 | 953 | 839 | 487 | 973 |
| Tmax | h | 0.083 | 0.083 | 0.083 | 0.083 | 0.083 |
| Cl_pred | L/(h*kg) | 0.00118 | 0.00112 | 0.00116 | 0.00212 | 0.00137 |
| HL_Lambda_z | h | 34.1 | 33.4 | 37.9 | 36.6 | 31.9 |
| (half life) | | | | | | |
| Vss_pred | L/kg | 0.0499 | 0.0488 | 0.0586 | 0.103 | 0.0574 |
| AUC_% Extrap_pred | (%) | 3 | 3 | 4 | 4 | 2 |
| AUC_% Back_Ext_pred | (%) | 0 | 0 | 0 | 0 | 0 |

As it can been seen in the Table 6 above and FIG. 5, the half-life of all five tested designed ankyrin proteins in mouse is over 30 h (32-38 h) and in a similar range.

### Example 5: Potency Assessment of Selected CD47 Specific Binding Proteins Using a SIRPα Signaling Assay

### Experiment A

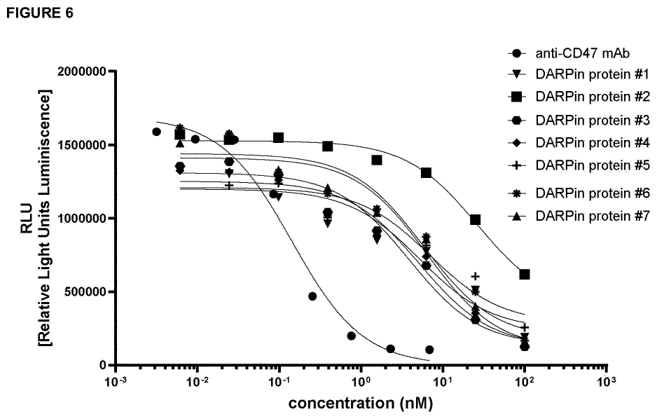

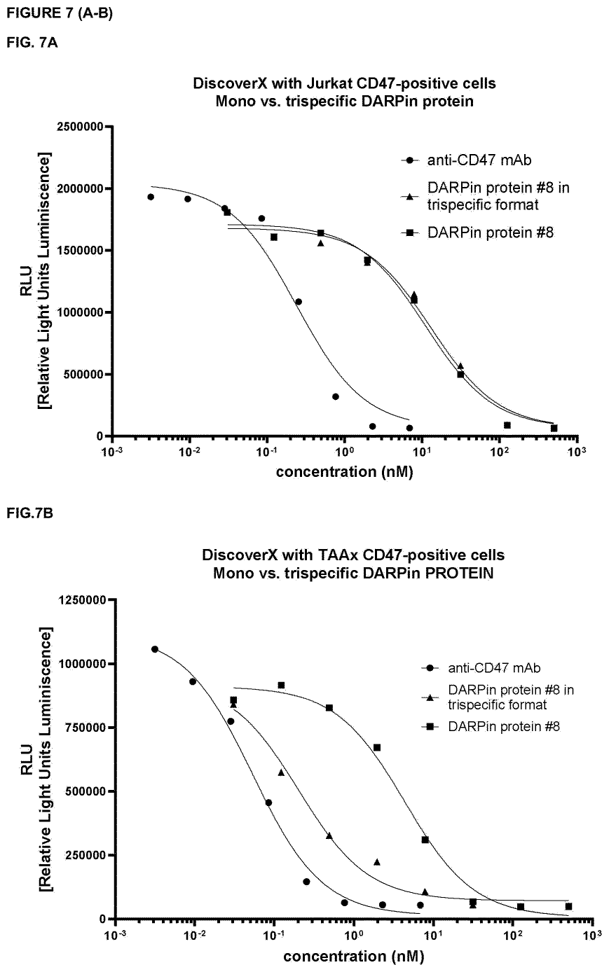

Selected CD47-specific designed ankyrin repeat proteins, formatted as trispecific proteins to also include (1) a designed ankyrin repeat domain specific for a tumor associated antigen (TAA) and (2) a designed ankyrin repeat domain specific for CD16a (SEQ ID NO: 81) (CD16a (UniProt (www.uniprot.org) Ref. No. P08637) is expressed on the surface of certain immune cells), were cloned, produced and tested for SIRP1α competition using the PathHunter Jurkat SIRPα Signaling Bioassay Kit as described above. Briefly, ligand engagement through co-culture of TAA positive/CD47 positive ligand cells and Jurkat SIRP1α signaling cells results in phosphorylation and intracellular pathway activation leading to a chemiluminescence signal. This signal can be inhibited in the presence of an anti-CD47 antibody or anti-SIRP1α antibody disrupting ligand-receptor interaction. As shown in FIGS. 6 and 7 and Table 7, the eight selected CD47-specific designed ankyrin repeat proteins in the trispecific format were able to inhibit ligand interaction. More particularly, FIG. 6 and Table 7 show the inhibition caused by CD47-specific DARPin proteins (SEQ ID NOs: 1 to 7), linked to a CD16a-specific designed ankyrin domain (SEQ ID NO: 81) and to a TAA-specific designed ankyrin repeat domain, upon addition to CD47 positive/TAA positive ligand cells and Jurkat SIRP1α signaling cells. FIG. 7A shows the inhibition caused by a CD47-specific DARPin protein (SEQ ID NO: 8) in the described trispecific format or in mono-domain (or mono-specific) format (DARPin protein #8) upon addition to the SIRP1α signaling cells in the presence of CD47 positive/TAA negative ligand cells. The CD47-specific DARPin protein (SEQ ID NO: 8) inhibited SIRP1α signaling equally well in the mono- or tri-specific formats in the presence of CD47 positive/TAA negative ligand cells. FIG. 7B shows the inhibition caused by a CD47-specific DARPin protein (SEQ ID NO: 8) in the described trispecific format or in mono-domain format (DARPin protein #8) upon addition to the SIRP1α signaling cells in the presence of CD47 positive/TAA positive ligand cells. The CD47-specific DARPin protein (SEQ ID NO: 8) inhibited SIRP1α signaling much stronger in the tri-specific format than in the mono-specific format in the presence of CD47 positive/TAA positive ligand cells. This enhanced inhibition of SIRP1α signaling by the trispecific construct suggests an enhancement due to avidity by the CD47-specific ankyrin repeat domain and the TAA-specific ankyrin repeat domain, which both bind to the surface of the CD47 positive/TAA positive ligand cells.

TABLE 7
Ligand inhibition (IC50 values) by CD47-
specific DARPins in trispecific format

| SEQ ID NO | IC50 (nM) |
|---|---|
| #1 | 5.2 |
| #2 | 26 |
| #6 | 3.9 |
| #5 | 5.2 |
| #3 | 7 |
| #4 | 5.8 |
| #7 | 6.4 |
| Benchmark anti-CD47 | 0.14 |
| monoclonal antibody-control | |

### Experiment B

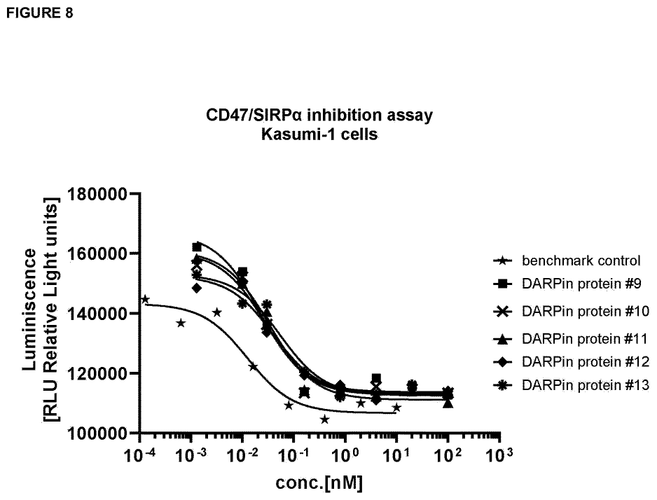

In a similar to the above described (Experiment A) experimental setup, five selected designed ankyrin repeat proteins in a trispecific format with binding specificity for CD47, CD117 (selection of DARPins with binding specificity for CD117 are disclosed in EP24150551, which is incorporated herein by reference) and CD16a were tested for SIRP1α competition using the PathHunter Jurkat SIRPα Signaling Bioassay Kit according to manufacturer's instructions. As shown in FIG. 8 and Table 8, DARPin protein #9 (SEQ ID NO: 94; including the ankyrin repeat domains of SEQ ID NO: 8, SEQ ID NO: 89 and SEQ ID NO: 81), DARPin protein #10 (SEQ ID NO: 95; including the ankyrin repeat domains of SEQ ID NO: 8, SEQ ID NO: 90 and SEQ ID NO: 81), DARPin protein #11 (SEQ ID NO: 96; including the ankyrin repeat domains of SEQ ID NO: 8, SEQ ID NO: 91 and SEQ ID NO: 81), DARPin protein #12 (SEQ ID NO: 97; including the ankyrin repeat domains of SEQ ID NO: 8, SEQ ID NO: 92 and SEQ ID NO: 81), and DARPin protein #13 (SEQ ID NO: 98; including the ankyrin repeat domains of SEQ ID NO: 8, SEQ ID NO: 93 and SEQ ID NO: 81) were able to inhibit ligand interaction, when added to the SIRP1α signaling cells in the presence of Kasumi-1 cells (these cells express both CD47 and CD117). DARPin proteins #9 to #13 show increased SIRP1α signaling inhibition when compared to DARPin protein #8 in the co-culture of Jurkat and Kasumi 1 cell lines, indicating the avidity effect caused by the CD47-specific ankyrin repeat domain and the CD117-specific ankyrin repeat domain, which both bind to the surface of the Kasumi-1 cells. As a control molecule, a benchmark anti-CD47 monoclonal antibody was used, having a bivalent structure.

TABLE 8
Ligand inhibition (IC50 values) by DARPin proteins
with binding specificity for CD47, CD117 and CD16a

| SEQ ID NO | IC50 (nM) |
|---|---|
| 94 (DARPin protein #9) | 0.022 |
| 95 (DARPin protein #10) | 0.028 |
| 96 (DARPin protein #11) | 0.034 |
| 97 (DARPin protein #12) | 0.04 |
| 98 (DARPin protein #13) | 0.054 |
| Benchmark anti-CD47 monoclonal | 0.013 |
| antibody control | |

The specification is most thoroughly understood in light of the teachings of the references cited within the specification. The aspects within the specification provide an illustration of aspects of the invention and should not be construed to limit the scope of the invention. The skilled artisan readily recognizes that many other aspects are encompassed by the invention. All publications, patents, and GenBank sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize or be able to ascertain using no more than routine experimentation, many equivalents to the specific aspects of the invention described herein. Such equivalents are intended to be encompassed by the following claims.

SEQUENCES

| SEQ | | |
|---|---|---|
| ID NO | Description | Sequence |
| 1 | Ankyrin repeat | ALGKKLLQAAARGQDDEVRELLKAGADVNAKDLIGRTPLHLAASIGHLEI |
| | domain specific | VEVLLKAGADVNAKDSRGWTPLHIAAVSGHLEIVEVLLKAGADVNAQDR |
| | for CD47 | AGATPADLAAAEGNEDIAEVLQKAA |
| 2 | Ankyrin repeat | DLGIKLLHAAFQGQDDEVRILLAAGADVNAKDKVGRTPLHRAAQWGHLE |
| | domain specific | IVEVLLKAGADVNAKDHVGWTPLHLAAWRGHLEIVEVLLKAGADVNAQD |
| | for CD47 | ASGFTPADLAARQGHEDIAEVLQKAA |
| 3 | Ankyrin repeat | DLGRKLLQAAARGQDDEVRELLKAGADVNAKDLHGRTPLHLAAAIGHLE |
| | domain specific | IVEVLLKAGADVNAKDFLGWTPLHIAAFRGHLEIVEVLLKAGADVNAQDE |
| | for CD47 | AGETPADLAAQEGHEDIAEVLQKAA |
| 4 | Ankyrin repeat | DLGKKLLQAAARGQDDEVRELLKAGADVNAKDLKGRTPLHLAAATGHP |
| | domain specific | EIVEVLLKAGADVNAKDQFGYTPLHIAAAWGHLEIVEVLLKAGADVNAQD |
| | for CD47 | QKGETPADLAAKEGHEDIAEVLQKAA |
| 5 | Ankyrin repeat | DLGRKLLQAAARGQDDEVRELLKAGADVNAKDLLGRTPLHLAASIGHLEI |
| | domain specific | VEVLLKAGADVNAKDERGFTPLHIAALQGHLEIVEVLLKAGADVNAQDLE |
| | for CD47 | GRTPADLAAEEGHEDIAEVLQKAA |
| 6 | Ankyrin repeat | DLGRKLLQAAARGQDDEVRELLKAGADVNAKDLHGRTPLHLAAAIGHLE |
| | domain specific | IVEVLLKAGADVNAKDVFGYTPLHIAAFRGHLEIVEVLLKAGADVNAQDE |
| | for CD47 | RGETPADLAAEAGHEDIAEVLQKAA |
| 7 | Ankyrin repeat | DLGEKLLKAAARGQDDEVRELLKAGADVNAKDLLGRTPLHLAASIGHLEI |
| | domain specific | VEVLLKAGADVNAKDERGYTPLHIAAIVGHLEIVEVLLKAGADVNAQDAR |
| | for CD47 | GATPADLAAREGHEDIAEVLQKAA |
| 8 | Ankyrin repeat | DLGKKLLQAAARGQDDEVRELLKAGADVNAKDLLGRTPLHLAASIGHLEI |
| | domain specific | VEVLLKAGADVNAKDSRGFTPLHLAAIIGHLEIVEVLLKAGADVNAQDLK |
| | for CD47 | GLTPADLAAAEGHEDIAEVLQKAA |
| 9 | Ankyrin repeat | KDLIGRTPLHLAASIGHLEIVEVLLKAGADVNA |
| | module | |
| 10 | Ankyrin repeat | KDSRGWTPLHIAAVSGHLEIVEVLLKAGADVNA |
| | module | |
| 11 | Ankyrin repeat | KDKVGRTPLHRAAQWGHLEIVEVLLKAGADVNA |
| | module | |
| 12 | Ankyrin repeat | KDHVGWTPLHLAAWRGHLEIVEVLLKAGADVNA |
| | module | |
| 13 | Ankyrin repeat | KDLHGRTPLHLAAAIGHLEIVEVLLKAGADVNA |
| | module | |
| 14 | Ankyrin repeat | KDFLGWTPLHIAAFRGHLEIVEVLLKAGADVNA |
| | module | |
| 15 | Ankyrin repeat | KDLKGRTPLHLAAATGHPEIVEVLLKAGADVNA |
| | module | |
| 16 | Ankyrin repeat | KDQFGYTPLHIAAAWGHLEIVEVLLKAGADVNA |
| | module | |
| 17 | Ankyrin repeat | KDLLGRTPLHLAASIGHLEIVEVLLKAGADVNA |
| | module | |
| 18 | Ankyrin repeat | KDERGFTPLHIAALQGHLEIVEVLLKAGADVNA |
| | module | |
| 19 | Ankyrin repeat | KDVFGYTPLHIAAFRGHLEIVEVLLKAGADVNA |
| | module | |
| 20 | Ankyrin repeat | KDERGYTPLHIAAIVGHLEIVEVLLKAGADVNA |
| | module | |
| 21 | Ankyrin repeat | KDSRGFTPLHLAAIIGHLEIVEVLLKAGADVNA |
| | module | |
| 22 | N-cap module | ALGKKLLQAAARGQDDEVRELLKAGADVNA |
| 23 | N-cap module | ALGKKLLQAAARGQDDEVRILLKAGADVNA |
| 24 | N-cap module | ALGKKLLQAAARGQDDEVRILLAAGADVNA |
| 25 | N-cap module | DLGIKLLHAAFQGQDDEVRILLAAGADVNA |
| 26 | N-cap module | DLGIKLLHAAFQGQDDEVRELLKAGADVNA |
| 27 | N-cap module | DLGIKLLHAAFQGQDDEVRILLKAGADVNA |
| 28 | N-cap module | DLGRKLLQAAARGQDDEVRELLKAGADVNA |
| 29 | N-cap module | DLGRKLLQAAARGQDDEVRILLAAGADVNA |
| 30 | N-cap module | DLGRKLLQAAARGQDDEVRILLKAGADVNA |
| 31 | N-cap module | DLGKKLLQAAARGQDDEVRELLKAGADVNA |
| 32 | N-cap module | DLGKKLLQAAARGQDDEVRILLAAGADVNA |
| 33 | N-cap module | DLGKKLLQAAARGQDDEVRILLKAGADVNA |
| 34 | N-cap module | DLGEKLLKAAARGQDDEVRELLKAGADVNA |
| 35 | N-cap module | DLGEKLLKAAARGQDDEVRILLAAGADVNA |
| 36 | N-cap module | DLGEKLLKAAARGQDDEVRILLKAGADVNA |
| 37 | C-cap module | QDRAGATPADLAAAEGNEDIAEVLQKAA |
| 38 | C-cap module | QDRAGATPADLAAAEGNEDIAEVLQKLA |
| 39 | C-cap module | QDRAGATPADLAAAEGNEDIAEVLQKLN |
| 40 | C-cap module | QDASGFTPADLAARQGHEDIAEVLQKAA |
| 41 | C-cap module | QDASGFTPADLAARQGHEDIAEVLQKLA |
| 42 | C-cap module | QDASGFTPADLAARQGHEDIAEVLQKLN |
| 43 | C-cap module | QDEAGETPADLAAQEGHEDIAEVLQKAA |
| 44 | C-cap module | QDEAGETPADLAAQEGHEDIAEVLQKLA |
| 45 | C-cap module | QDEAGETPADLAAQEGHEDIAEVLQKLN |
| 46 | C-cap module | QDQKGETPADLAAKEGHEDIAEVLQKAA |
| 47 | C-cap module | QDQKGETPADLAAKEGHEDIAEVLQKLA |
| 48 | C-cap module | QDQKGETPADLAAKEGHEDIAEVLQKLN |
| 49 | C-cap module | QDLEGRTPADLAAEEGHEDIAEVLQKAA |
| 50 | C-cap module | QDLEGRTPADLAAEEGHEDIAEVLQKLA |
| 51 | C-cap module | QDLEGRTPADLAAEEGHEDIAEVLQKLN |
| 52 | C-cap module | QDERGETPADLAAEAGHEDIAEVLQKAA |
| 53 | C-cap module | QDERGETPADLAAEAGHEDIAEVLQKLA |
| 54 | C-cap module | QDERGETPADLAAEAGHEDIAEVLQKLN |
| 55 | C-cap module | QDARGATPADLAAREGHEDIAEVLQKAA |
| 56 | C-cap module | QDARGATPADLAAREGHEDIAEVLQKLA |
| 57 | C-cap module | QDARGATPADLAAREGHEDIAEVLQKLN |
| 58 | C-cap module | QDLKGLTPADLAAAEGHEDIAEVLQKAA |
| 59 | C-cap module | QDLKGLTPADLAAAEGHEDIAEVLQKLA |
| 60 | C-cap module | QDLKGLTPADLAAAEGHEDIAEVLQKLN |
| 61 | His Tag | MRGSHHHHHHGS |
| 62 | Ankyrin repeat | DLGKKLLEAARAGQDDEVRELLKAGADVNAKDYFSHTPLHLAARNGHL |
| | domain specific | KIVEVLLKAGADVNAKDFAGKTPLHLAANEGHLEIVEVLLKAGADVNAQD |
| | for human serum | IFGKTPADIAADAGHEDIAEVLQKAA |
| | albumin | |
| 63 | Ankyrin repeat | DLGKKLLEAARAGQDDEVRELLKAGADVNAKDYFSHTPLHLAARNGHL |
| | domain specific | KIVEVLLKAGADVNAKDFAGKTPLHLAAADGHLEIVEVLLKAGADVNAQD |
| | for human serum | IFGKTPADIAADAGHEDIAEVLQKAA |
| | albumin | |
| 64 | Ankyrin repeat | DLGKKLLEAARAGQDDEVRELLKAGADVNAKDYFSHTPLHLAARNGHL |
| | domain specific | KIVEVLLKAGADVNAKDFAGKTPLHLAADAGHLEIVEVLLKAGADVNAQD |
| | for human serum | IFGKTPADIAADAGHEDIAEVLQKAA |
| | albumin | |
| 65 | PT linker | GSPTPTPTTPTPTPTTPTPTPT |
| 66 | Consensus GS | [GGGGS]n, wherein n is 1, 2, 3, 4, 5, or 6 |
| | linker | |
| 67 | Bio-Human | QLLFNKTKSVEFTFCNDTVVIPCFVTNMEAQNTTEVYVKWKFKGRDIYTF |
| | CD47-Fc | DGALNKSTVPTDFSSAKIEVSQLLKGDASLKMDKSDAVSHTGNYTCEVT |
| | | ELTREGETIIELKYRVVSWFSPIEGRMDPKSCDKTHTCPPCPAPELLGGP |
| | | SVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTI |
| | | SKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |
| 68 | Ankyrin repeat | DLGKKLLEAARAGQDDEVRELLKAGADVNAKDYFSHTPLHLAARNGHL |
| | protein specific for | KIVEVLLKAGADVNAKDFAGKTPLHLAAADGHLEIVEVLLKAGADVNAQD |
| | CD47 and human | IFGKTPADIAADAGHEDIAEVLQKAAGSPTPTPTTPTPTPTTPTPTPTGSD |
| | serum albumin | LGRKLLQAAARGQDDEVRELLKAGADVNAKDLHGRTPLHLAAAIGHLEI |
| | | VEVLLKAGADVNAKDVFGYTPLHIAAFRGHLEIVEVLLKAGADVNAQDER |
| | | GETPADLAAEAGHEDIAEVLQKAA |
| 69 | Ankyrin repeat | DLGKKLLEAARAGQDDEVRELLKAGADVNAKDYFSHTPLHLAARNGHL |
| | protein specific for | KIVEVLLKAGADVNAKDFAGKTPLHLAAADGHLEIVEVLLKAGADVNAQD |
| | CD47 and human | IFGKTPADIAADAGHEDIAEVLQKAAGSPTPTPTTPTPTPTTPTPTPTGSD |
| | serum albumin | LGKKLLQAAARGQDDEVRELLKAGADVNAKDLKGRTPLHLAAATGHPEI |
| | | VEVLLKAGADVNAKDQFGYTPLHIAAAWGHLEIVEVLLKAGADVNAQDQ |
| | | KGETPADLAAKEGHEDIAEVLQKAA |
| 70 | Ankyrin repeat | DLGKKLLEAARAGQDDEVRELLKAGADVNAKDYFSHTPLHLAARNGHL |
| | protein specific for | KIVEVLLKAGADVNAKDFAGKTPLHLAAADGHLEIVEVLLKAGADVNAQD |
| | CD47 and human | IFGKTPADIAADAGHEDIAEVLQKAAGSPTPTPTTPTPTPTTPTPTPTGSD |
| | serum albumin | LGEKLLKAAARGQDDEVRELLKAGADVNAKDLLGRTPLHLAASIGHLEIV |
| | | EVLLKAGADVNAKDERGYTPLHIAAIVGHLEIVEVLLKAGADVNAQDARG |
| | | ATPADLAAREGHEDIAEVLQKAA |
| 71 | Ankyrin repeat | DLGKKLLEAARAGQDDEVRELLKAGADVNAKDYFSHTPLHLAARNGHL |
| | protein specific for | KIVEVLLKAGADVNAKDFAGKTPLHLAAADGHLEIVEVLLKAGADVNAQD |
| | CD47 and human | IFGKTPADIAADAGHEDIAEVLQKAAGSPTPTPTTPTPTPTTPTPTPTGSD |
| | serum albumin | LGRKLLQAAARGQDDEVRELLKAGADVNAKDLLGRTPLHLAASIGHLEIV |
| | | EVLLKAGADVNAKDERGFTPLHIAALQGHLEIVEVLLKAGADVNAQDLEG |
| | | RTPADLAAEEGHEDIAEVLQKAA |
| 72 | Ankyrin repeat | DLGKKLLEAARAGQDDEVRELLKAGADVNAKDYFSHTPLHLAARNGHL |
| | protein specific for | KIVEVLLKAGADVNAKDFAGKTPLHLAAADGHLEIVEVLLKAGADVNAQD |
| | CD47 and human | IFGKTPADIAADAGHEDIAEVLQKAAGSPTPTPTTPTPTPTTPTPTPTGSD |
| | serum albumin | LGKKLLQAAARGQDDEVRELLKAGADVNAKDLLGRTPLHLAASIGHLEIV |
| | | EVLLKAGADVNAKDSRGFTPLHLAAIIGHLEIVEVLLKAGADVNAQDLKGL |
| | | TPADLAAAEGHEDIAEVLQKAA |
| 73 | Nucleic acid | GCCCTGGGTAAGAAGCTGCTGCAAGCTGCTGCTCGTGGTCAGGATG |
| | encoding domain | ATGAAGTTCGTGAACTGCTGAAAGCAGGCGCCGATGTTAATGCAAAA |
| | of SEQ ID NO 1 | GATCTGATCGGCAGAACCCCGCTGCATCTGGCTGCTTCTATCGGTCA |
| | | CCTGGAAATTGTTGAAGTTCTGCTGAAAGCCGGTGCAGATGTTAATG |
| | | CAAAAGATTCTAGAGGCTGGACCCCGCTGCATATCGCTGCTGTGTCT |
| | | GGTCACCTGGAAATTGTTGAAGTTCTGCTGAAAGCCGGTGCAGATGT |
| | | TAACGCACAGGATCGTGCTGGCGCTACCCCTGCCGATCTGGCAGCT |
| | | GCTGAAGGTAATGAAGATATTGCAGAAGTGCTGCAGAAGGCAGCG |
| 74 | Nucleic acid | GACCTGGGTATCAAACTGCTGCATGCTGCTTTCCAGGGTCAGGACG |
| | encoding domain | ACGAAGTTCGTATCCTGCTGGCTGCTGGCGCCGACGTTAATGCTAAA |
| | of SEQ ID NO 2 | GACAAAGTTGGTCGTACTCCGCTGCACCGTGCTGCTCAGTGGGGTC |
| | | ACCTGGAAATCGTTGAAGTTCTACTGAAGGCTGGTGCTGACGTTAAT |
| | | GCTAAAGACCATGTTGGTTGGACTCCGCTGCACCTGGCTGCTTGGC |
| | | GTGGTCACCTGGAAATCGTTGAAGTTCTGCTGAAGGCTGGTGCTGAC |
| | | GTTAACGCTCAGGACGCTTCTGGTTTCACTCCGGCTGATCTGGCTGC |
| | | TCGTCAGGGTCACGAGGATATCGCTGAAGTTCTGCAGAAGGCAGCG |
| 75 | Nucleic acid | GACCTGGGTCGTAAGCTGCTGCAAGCTGCTGCTCGTGGTCAGGATG |
| | encoding domain | ATGAAGTTCGTGAACTGCTGAAAGCAGGCGCCGATGTTAATGCAAAA |
| | of SEQ ID NO 3 | GATCTGCACGGCAGAACCCCGCTGCATCTGGCTGCTGCTATCGGTC |
| | | ACCTGGAAATTGTTGAAGTTCTGCTGAAAGCCGGTGCAGATGTTAAT |
| | | GCAAAAGATTTCCTGGGCTGGACCCCGCTGCATATCGCTGCTTTCAG |
| | | AGGTCACCTGGAAATTGTTGAAGTTCTGCTGAAAGCCGGTGCAGATG |
| | | TTAACGCACAGGATGAAGCTGGCGAAACCCCTGCCGATCTGGCAGC |
| | | TCAGGAAGGTCATGAAGATATTGCAGAAGTGCTGCAGAAGGCAGCG |
| 76 | Nucleic acid | GACCTGGGTAAGAAGCTGCTGCAAGCTGCTGCTCGTGGTCAGGATG |
| | encoding domain | ATGAAGTTCGTGAACTGCTGAAAGCAGGCGCCGATGTTAATGCAAAA |
| | of SEQ ID NO 4 | GATCTGAAGGGCAGAACCCCGCTGCATCTGGCTGCTGCTACCGGTC |
| | | ACCCGGAAATTGTTGAAGTTCTGCTGAAAGCCGGTGCAGATGTTAAT |
| | | GCAAAAGATCAATTCGGCTACACCCCGCTGCATATCGCTGCTGCTTG |
| | | GGGTCACCTGGAAATTGTTGAAGTTCTGCTGAAAGCCGGTGCAGATG |
| | | TTAACGCACAGGATCAGAAGGGCGAAACCCCTGCCGATCTGGCAGC |
| | | TAAGGAAGGTCATGAAGATATTGCAGAAGTGCTGCAGAAGGCAGCG |
| 77 | Nucleic acid | GACCTGGGTCGTAAGCTGCTGCAAGCTGCTGCTCGTGGTCAGGATG |
| | encoding domain | ATGAAGTTCGTGAACTGCTGAAAGCAGGCGCCGATGTTAATGCAAAA |
| | of SEQ ID NO 5 | GATCTGCTGGGCAGAACCCCGCTGCATCTGGCTGCTTCTATCGGTC |
| | | ACCTGGAAATTGTTGAAGTTCTGCTGAAAGCCGGTGCAGATGTTAAT |
| | | GCAAAAGATGAGAGAGGCTTCACCCCGCTGCATATCGCTGCTTTACA |
| | | AGGTCACCTGGAAATTGTTGAAGTTCTGCTGAAAGCCGGTGCAGATG |
| | | TTAACGCACAGGATCTGGAAGGCCGTACCCCTGCCGATCTGGCAGC |
| | | TGAAGAAGGTCATGAAGATATTGCAGAAGTGCTGCAGAAGGCAGCG |
| 78 | Nucleic acid | GACCTGGGTCGTAAGCTGCTGCAAGCTGCTGCTCGTGGTCAGGATG |
| | encoding domain | ATGAAGTTCGTGAACTGCTGAAAGCAGGCGCCGATGTTAATGCAAAA |
| | of SEQ ID NO 6 | GATCTGCACGGCAGAACCCCGCTGCATCTGGCTGCTGCTATCGGTC |
| | | ACCTGGAAATTGTTGAAGTTCTGCTGAAAGCCGGTGCAGATGTTAAT |
| | | GCAAAAGATGTGTTCGGCTACACCCCGCTGCATATCGCTGCCTTCAG |
| | | AGGTCACCTGGAAATTGTTGAAGTTCTGCTGAAAGCCGGTGCAGATG |
| | | TTAACGCACAGGATGAACGTGGCGAAACCCCTGCCGATCTGGCAGC |
| | | TGAAGCTGGTCATGAAGATATTGCAGAAGTGCTGCAGAAGGCAGCG |
| 79 | Nucleic acid | GACCTGGGTGAAAAGCTGCTGAAGGCTGCTGCTCGTGGTCAGGATG |
| | encoding domain | ATGAAGTTCGTGAACTGCTGAAAGCAGGCGCCGATGTTAATGCAAAA |
| | of SEQ ID NO 7 | GATCTGCTGGGCAGAACCCCGCTGCATCTGGCTGCTTCTATCGGTC |
| | | ACCTGGAAATTGTTGAAGTTCTGCTGAAAGCCGGTGCAGATGTTAAT |
| | | GCAAAAGATGAGAGAGGCTACACCCCGCTGCATATCGCTGCTATCGT |
| | | GGGTCACCTGGAAATTGTTGAAGTTCTGCTGAAAGCCGGTGCAGATG |
| | | TTAACGCACAGGATGCTCGTGGCGCTACCCCTGCCGATCTGGCAGC |
| | | TCGTGAAGGTCATGAAGATATTGCAGAAGTGCTGCAGAAGGCAGCG |
| 80 | Nucleic acid | GACCTGGGTAAGAAGCTGCTGCAAGCTGCTGCTCGTGGTCAGGATG |
| | encoding domain | ATGAAGTTCGTGAACTGCTGAAAGCAGGCGCCGATGTTAATGCAAAA |
| | of SEQ ID NO 8 | GATCTGCTGGGCAGAACCCCGCTGCATCTGGCTGCTTCTATCGGTC |
| | | ACCTGGAAATTGTTGAAGTTCTGCTGAAAGCCGGTGCAGATGTTAAT |
| | | GCAAAAGATTCTAGAGGCTTCACCCCGCTGCATCTGGCTGCTATCAT |
| | | CGGTCACCTGGAAATTGTTGAAGTTCTGCTGAAAGCCGGTGCAGATG |
| | | TTAACGCACAGGATCTGAAGGGCCTGACCCCTGCCGATCTGGCAGC |
| | | TGCTGAAGGTCATGAAGATATTGCAGAAGTGCTGCAGAAGGCAGCG |
| 81 | Ankyrin repeat | DLGQKLLRAAKKGQDDEVRELLKAGADVNAKDEQGWTPLHIAAWKGHL |
| | domain specific | EIVEVLLKAGADVNAKDVFDWTPLHVAAHFGHLEIVEVLLKAGADVNAQ |
| | for CD16a | DRAGETPADLAAQAGHEDIAEVLQKAA |
| 82 | N-cap module | DLGXKLLQAAXXGQLDXVRXLLXAGADVNA |
| | randomized | |
| 83 | N-cap module | DLGXXLLQAAXXGQLDXVRXLXXXGADVNA |
| | randomized | |
| 84 | N-cap module | XXXXXLLQAAXXGXXXXXXXXXXXGADVNA |
| | randomized | |
| 85 | N-cap module | DLGXKLLEAAXXGQDDXVRXLLXAGADVNA |
| | randomized | |
| 86 | N-cap module | DLGXXLLEAAXXGQDDXVRXLXXXGADVNA |
| | randomized | |
| 87 | N-cap module | XXXXXLLXAAXXGXDXXXXXXXXXGADVNA |
| | randomized | |
| 88 | C-cap module | XDXXGXTPADXAADXGHEXIAXVLQXAA |
| | randomized | |
| 89 | Ankyrin repeat | DLGQKLLKAAQYGQDDEVRILLAAGADVNAKDWAGKTPLHVAAEAGHL |
| | protein with | EIVEVLLKAGADVNAKDKAGETPLHLAAEQGHLEIVEVLLKAGADVNAQD |
| | binding specificity | EWGQTPADLAAYYGLEDIAEVLQKAA |
| | for CD117 | |
| 90 | Ankyrin repeat | DLGAKLLKAAQSGQDDEVRILLAAGADVNAKDKDGYTPLHLAAREGHLE |
| | protein with | IVEVLLKAGSDVNAKDYYGETPLHQAAEYGHLEIVEVLLKAGADVNAQD |
| | | EWGATPADLAAQQGHEDIAEVLQKAA |
| | binding specificity | |
| | for CD117 | |
| 91 | Ankyrin repeat | DLGKKLLQAARAGQLDEVRELLKAGADVNAKDVYGLTPLHLAAREGHLE |
| | protein with | IVEVLLKAGADVNAKDAYGETPLHLAAEAGHLEIVEVLLKAGADVNAKDE |
| | binding specificity | WGETPLNVAARAGHQDIAEVLQKAA |
| | for CD117 | |
| 92 | Ankyrin repeat | DLGKKLLKAASWGQDDEVRILLAAGADVNAKDNSGWTPLHAAAWYGHL |
| | protein with | EIVEVLLKAGADVNAKDKTGLTPLHKAAHVGHLEIVEVLLKAGADVNAQD |
| | binding specificity | EWGATPADLAASTGHEDIAEVLQKAA |
| | for CD117 | |
| 93 | Ankyrin repeat | DLGHKLLHAAAWGQDDEVRILLAAGADVNAKDQSGWTPLHVAAWYGH |
| | protein with | LEIVEVLLKAGADVNAKDLKGETPLHLAALVGHLEIVEVLLKAGADVNAQ |
| | binding specificity | DIAGDTPADLAAIAGHEDIAEVLQKAA |
| | for CD117 | |
| 94 | Ankyrin repeat | DLGKKLLQAAARGQDDEVRELLKAGADVNAKDLLGRTPLHLAASIGHLEI |
| | protein with | VEVLLKAGADVNAKDSRGFTPLHLAAIIGHLEIVEVLLKAGADVNAQDLK |
| | binding specificity | GLTPADLAAAEGHEDIAEVLQKAAGSPTPTPTTPTPTPTTPTPTPTTPTP |
| | for CD47-CD117- | TPTTPTPTPTGSDLGQKLLKAAQYGQDDEVRILLAAGADVNAKDWAGKT |
| | CD16a | PLHVAAEAGHLEIVEVLLKAGADVNAKDKAGETPLHLAAEQGHLEIVEVL |
| | | LKAGADVNAQDEWGQTPADLAAYYGLEDIAEVLQKAAGSPTPTPTTPTP |
| | | TPTTPTPTPTGSDLGQKLLRAAKKGQDDEVRELLKAGADVNAKDEQGW |
| | | TPLHIAAWKGHLEIVEVLLKAGADVNAKDVFDWTPLHVAAHFGHLEIVEV |
| | | LLKAGADVNAQDRAGETPADLAAQAGHEDIAEVLQKAA |
| 95 | Ankyrin repeat | DLGKKLLQAAARGQDDEVRELLKAGADVNAKDLLGRTPLHLAASIGHLEI |
| | protein with | VEVLLKAGADVNAKDSRGFTPLHLAAIIGHLEIVEVLLKAGADVNAQDLK |
| | binding specificity | GLTPADLAAAEGHEDIAEVLQKAAGSPTPTPTTPTPTPTTPTPTPTTPTP |
| | for CD47-CD117- | TPTTPTPTPTGSDLGAKLLKAAQSGQDDEVRILLAAGADVNAKDKDGYT |
| | CD16a | PLHLAAREGHLEIVEVLLKAGSDVNAKDYYGETPLHQAAEYGHLEIVEVL |
| | | LKAGADVNAQDEWGATPADLAAQQGHEDIAEVLQKAAGSPTPTPTTPT |
| | | PTPTTPTPTPTGSDLGQKLLRAAKKGQDDEVRELLKAGADVNAKDEQG |
| | | WTPLHIAAWKGHLEIVEVLLKAGADVNAKDVFDWTPLHVAAHFGHLEIV |
| | | EVLLKAGADVNAQDRAGETPADLAAQAGHEDIAEVLQKAA |
| 96 | Ankyrin repeat | DLGKKLLQAAARGQDDEVRELLKAGADVNAKDLLGRTPLHLAASIGHLEI |
| | protein with | VEVLLKAGADVNAKDSRGFTPLHLAAIIGHLEIVEVLLKAGADVNAQDLK |
| | binding specificity | GLTPADLAAAEGHEDIAEVLQKAAGSPTPTPTTPTPTPTTPTPTPTTPTP |
| | for CD47-CD117- | TPTTPTPTPTGSDLGKKLLQAARAGQLDEVRELLKAGADVNAKDVYGLT |
| | CD16a | PLHLAAREGHLEIVEVLLKAGADVNAKDAYGETPLHLAAEAGHLEIVEVL |
| | | LKAGADVNAKDEWGETPLNVAARAGHQDIAEVLQKAAGSPTPTPTTPT |
| | | PTPTTPTPTPTGSDLGQKLLRAAKKGQDDEVRELLKAGADVNAKDEQG |
| | | WTPLHIAAWKGHLEIVEVLLKAGADVNAKDVFDWTPLHVAAHFGHLEIV |
| | | EVLLKAGADVNAQDRAGETPADLAAQAGHEDIAEVLQKAA |
| 97 | Ankyrin repeat | DLGKKLLQAAARGQDDEVRELLKAGADVNAKDLLGRTPLHLAASIGHLEI |
| | protein with | VEVLLKAGADVNAKDSRGFTPLHLAAIIGHLEIVEVLLKAGADVNAQDLK |
| | binding specificity | GLTPADLAAAEGHEDIAEVLQKAAGSPTPTPTTPTPTPTTPTPTPTTPTP |
| | for CD47-CD117- | TPTTPTPTPTGSDLGKKLLKAASWGQDDEVRILLAAGADVNAKDNSGW |
| | CD16a | TPLHAAAWYGHLEIVEVLLKAGADVNAKDKTGLTPLHKAAHVGHLEIVEV |
| | | LLKAGADVNAQDEWGATPADLAASTGHEDIAEVLQKAAGSPTPTPTTPT |
| | | PTPTTPTPTPTGSDLGQKLLRAAKKGQDDEVRELLKAGADVNAKDEQG |
| | | WTPLHIAAWKGHLEIVEVLLKAGADVNAKDVFDWTPLHVAAHFGHLEIV |
| | | EVLLKAGADVNAQDRAGETPADLAAQAGHEDIAEVLQKAA |
| 98 | Ankyrin repeat | DLGKKLLQAAARGQDDEVRELLKAGADVNAKDLLGRTPLHLAASIGHLEI |
| | protein with | VEVLLKAGADVNAKDSRGFTPLHLAAIIGHLEIVEVLLKAGADVNAQDLK |
| | binding specificity | GLTPADLAAAEGHEDIAEVLQKAAGSPTPTPTTPTPTPTTPTPTPTTPTP |
| | or CD47-CD117- | TPTTPTPTPTGSDLGHKLLHAAAWGQDDEVRILLAAGADVNAKDQSGW |
| | CD16a | TPLHVAAWYGHLEIVEVLLKAGADVNAKDLKGETPLHLAALVGHLEIVEV |
| | | LLKAGADVNAQDIAGDTPADLAAIAGHEDIAEVLQKAAGSPTPTPTTPTP |
| | | TPTTPTPTPTGSDLGQKLLRAAKKGQDDEVRELLKAGADVNAKDEQGW |
| | | TPLHIAAWKGHLEIVEVLLKAGADVNAKDVFDWTPLHVAAHFGHLEIVEV |
| | | LLKAGADVNAQDRAGETPADLAAQAGHEDIAEVLQKAA |

1. A recombinant binding protein comprising an ankyrin repeat domain with binding specificity for CD47, wherein said ankyrin repeat domain comprises an ankyrin repeat module having an amino acid sequence selected from the group consisting of (1) SEQ ID NOS: 9 to 21 and (2) sequences in which up to 9 amino acids in any of SEQ ID NOs: 9 to 21 are substituted by other amino acids.

2. The recombinant binding protein of claim 1, wherein said ankyrin repeat module is a first ankyrin repeat module and wherein said ankyrin repeat domain further comprises a second ankyrin repeat module having an amino acid sequence selected from the group consisting of (1) SEQ ID NOS: 9 to 21 and (2) sequences in which up to 9 amino acids in any of SEQ ID NOs: 9 to 21 are substituted by other amino acids.

3. The recombinant binding protein of claim 2, wherein
i. said first ankyrin repeat module comprises an amino acid sequence selected from the group consisting of (1) SEQ ID NO: 9 and (2) sequences in which up to 9 amino acids in SEQ ID NO: 9 are substituted by other amino acids, and wherein said second ankyrin repeat module comprises an amino acid sequence selected from the group consisting of (1) SEQ ID NO: 10 and (2) sequences in which up to 9 amino acids of SEQ ID NO: 10 are substituted by other amino acids;
ii. said first ankyrin repeat module comprises an amino acid sequence selected from the group consisting of (1) SEQ ID NO: 11 and (2) sequences in which up to 9 amino acids in SEQ ID NO: 11 are substituted by other amino acids, and wherein said second ankyrin repeat module comprises an amino acid sequence selected from the group consisting of (1) SEQ ID NO: 12 and (2) sequences in which up to 9 amino acids of SEQ ID NO: 12 are substituted by other amino acids;
iii. said first ankyrin repeat module comprises an amino acid sequence selected from the group consisting of (1) SEQ ID NO: 13 and (2) sequences in which up to 9 amino acids in SEQ ID NO: 13 are substituted by other amino acids, and wherein said second ankyrin repeat module comprises an amino acid sequence selected from the group consisting of (1) SEQ ID NO: 14 and (2) sequences in which up to 9 amino acids of SEQ ID NO: 14 are substituted by other amino acids;
iv. said first ankyrin repeat module comprises an amino acid sequence selected from the group consisting of (1) SEQ ID NO: 15 and (2) sequences in which up to 9 amino acids in SEQ ID NO: 15 are substituted by other amino acids, and wherein said second ankyrin repeat module comprises an amino acid sequence selected from the group consisting of (1) SEQ ID NO:16 and (2) sequences in which up to 9 amino acids of SEQ ID NO: 16 are substituted by other amino acids;
v. said first ankyrin repeat module comprises an amino acid sequence selected from the group consisting of (1) SEQ ID NO: 17 and (2) sequences in which up to 9 amino acids in SEQ ID NO: 17 are substituted by other amino acids, and wherein said second ankyrin repeat module comprises an amino acid sequence selected from the group consisting of (1) SEQ ID NO: 18 and (2) sequences in which up to 9 amino acids of SEQ ID NO: 18 are substituted by other amino acids;
vi. said first ankyrin repeat module comprises an amino acid sequence selected from the group consisting of (1) SEQ ID NO: 13 and (2) sequences in which up to 9 amino acids in SEQ ID NO: 13 are substituted by other amino acids, and wherein said second ankyrin repeat module comprises an amino acid sequence selected from the group consisting of (1) SEQ ID NO: 19 and (2) sequences in which up to 9 amino acids of SEQ ID NO: 19 are substituted by other amino acids;
vii. said first ankyrin repeat module comprises an amino acid sequence selected from the group consisting of (1) SEQ ID NO: 17 and (2) sequences in which up to 9 amino acids in SEQ ID NO: 17 are substituted by other amino acids, and wherein said second ankyrin repeat module comprises an amino acid sequence selected from the group consisting of (1) SEQ ID NO: 20 and (2) sequences in which up to 9 amino acids of SEQ ID NO: 20 are substituted by other amino acids; or
viii. said first ankyrin repeat module comprises an amino acid sequence selected from the group consisting of (1) SEQ ID NO: 17 and (2) sequences in which up to 9 amino acids in SEQ ID NO: 17 are substituted by other amino acids, and wherein said second ankyrin repeat module comprises an amino acid sequence selected from the group consisting of (1) SEQ ID NO: 21 and (2) sequences in which up to 9 amino acids of SEQ ID NO: 21 are substituted by other amino acids.

4. The recombinant binding protein of claim 2 or 3, wherein said first ankyrin repeat module is located N-terminally of said second ankyrin repeat module within said ankyrin repeat domain.

5. A recombinant binding protein comprising an ankyrin repeat domain with binding specificity for CD47, wherein said ankyrin repeat domain comprises an amino acid sequence with at least 80% amino acid sequence identity with any one of SEQ ID NOS: 1 to 8, and wherein A at the second last position of SEQ ID NOS: 1 to 8 is optionally substituted by L, and/or A at the last position of SEQ ID NOs: 1 to 8 is optionally substituted by N.

6. The recombinant binding protein of any one of the preceding claims, wherein said recombinant binding protein binds human CD47 in PBS with a dissociation constant (K_{D}) of or below 10⁻⁷M, preferably of or below 10⁻⁸M, even more preferably of or below 10⁻⁹M.

7. The recombinant binding protein of any one of the preceding claims, wherein said recombinant binding protein further comprises a binding agent with binding specificity for a tumor-associated antigen, optionally wherein said binding agent with binding specificity for a tumor-associated antigen is an ankyrin repeat domain.

8. The recombinant binding protein of any one of the preceding claims, wherein said recombinant binding protein further comprises a binding agent with binding specificity for a protein expressed on the surface of an immune cell.

9. The recombinant binding protein of claim 8, wherein said protein expressed on the surface of an immune cell is CD16a, optionally wherein said binding agent with binding specificity for a protein expressed on the surface of an immune cell is an ankyrin repeat domain, optionally wherein said binding agent with binding specificity for a protein expressed on the surface of an immune cell comprises an amino acid sequence with at least 80% amino acid sequence identity with SEQ ID NO: 81.

10. The recombinant binding protein of any one of the preceding claims, wherein said recombinant binding protein further comprises a half-life extending moiety.

11. The recombinant binding protein of claim 10, wherein said half-life extending moiety comprises a binding agent with binding specificity for human serum albumin, optionally wherein said binding agent with binding specificity for human serum albumin is an ankyrin repeat domain, optionally wherein said binding agent with binding specificity for human serum albumin comprises an amino acid sequence with at least 80% amino acid sequence identity with any one of SEQ ID NOs: 62 to 64.

12. A nucleic acid encoding the recombinant binding protein of any of the preceding claims.

13. A pharmaceutical composition comprising the recombinant binding protein of any one of claims 1 to 11 or the nucleic acid of claim 12, and optionally a pharmaceutically acceptable carrier and/or diluent.

14. A method of treating a medical condition, the method comprising the step of administering to a subject in need thereof a therapeutically effective amount of the recombinant binding protein of any one of claims 1 to 11, the nucleic acid of claim 12 or the pharmaceutical composition of claim 13.

15. The method of claim 14, wherein said medical condition is cancer.
